(19) United States
(12) Patent Application Publication
(10) Pub. No.: US 2025/0375445 A1
(43) Pub. Date: Dec. 11, 2025

(54) METHODS OF TREATING A RAS PROTEIN-RELATED DISEASE OR DISORDER

(71) Applicant: Revolution Medicines, Inc., Redwood City, CA (US)

(72) Inventors: Jingjing JIANG, Redwood City, CA (US); Benjamin MALDONATO, Los Gatos, CA (US); Mallika SINGH, San Francisco, CA (US); Zhengping WANG, San Mateo, CA (US); Zhican WANG, San Mateo, CA (US); Michael FLAGELLA, Oakland, CA (US); W. Clay GUSTAFSON, San Francisco, CA (US)

(21) Appl. No.: 19/230,256

(22) Filed: Jun. 6, 2025

Related U.S. Application Data
(60) Provisional application No. 63/657,527, filed on Jun. 7, 2024.
(60) Provisional application No. 63/662,775, filed on Jun. 21, 2024.

(51) Int. Cl. A61K 31/504; A61K 45/06; A61P 35/00

(52) U.S. Cl. CPC A61K 31/504; A61K 45/06; A61P 35/00

(57) ABSTRACT

Disclosed are RAS(ON) multi-selective inhibitor compositions and methods of treating RAS protein-related diseases or disorders using an intermittent dosing regimen. RAS(ON) multi-selective inhibitors having tight binding to cyclophilin A (CypA) result in high exposure levels, prolonged tissue retention, and/or slow clearance rates, thereby increasing the risk of inhibition of wild-type RAS in normal tissues. Accordingly, provided herein are methods for the safe and effective dosing of low K_{D}1 RAS(ON) multi-selective inhibitors using an intermittent dosing regimen. Also provided are methods of selecting or identifying such RAS(ON) multi-selective inhibitors suitable for intermittent administration.

### BACKGROUND

It has been well established in literature that RAS proteins (K-RAS, H-RAS, and N-RAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13C) and 61 (e.g., Q61K) of RAS are also responsible for oncogenic activity in some cancers.

In normal cells, RAS proteins play a critical role in regulating cell growth, differentiation, and survival, acting as molecular switches, relaying signals from cell surface receptors to intracellular pathways that control key cellular processes. Genetic studies have demonstrated that complete deletion of RAS genes is lethal in mouse models and the absence of cellular proliferation in vitro (Drosten et al. Oncogene 33, 2857-2865 (2014); Drosten et al. EMBO J. 29, 1091-1104 (2010)). Furthermore, KRAS conditional knockout in adult bone marrow has been shown to induce significant hematopoietic defects, including splenomegaly, an expanded neutrophil compartment, and reduced B cell number (Zhang et. al., Stem Cells; 34(7):1859-71 (2016)).

There remains a need for effective and/or enhanced treatment methods for individuals suffering the effects of a RAS mutation.

### SUMMARY OF THE INVENTION

In an aspect, the invention features a method of treating a RAS protein-related disease in a subject in need thereof, the method including administering to the subject a RAS(ON) multi-selective inhibitor on an intermittent dosing regimen.

In some embodiments, the subject has a mutation of RAS. In some embodiments, the mutation of RAS is a KRAS mutation.

In some embodiments, the RAS protein-related disease is cancer. In some embodiments, the cancer is lung cancer, pancreatic cancer, or colorectal cancer.

In some embodiments, the RAS(ON) multi-selective inhibitor has a blood or plasma half-life of 12 hours or longer (e.g., 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, or 50 hours).

In some embodiments, the RAS(ON) multi-selective inhibitor has a K_{D}1 of 0.1 nM to 500 nM or lower. In some embodiments, the RAS(ON) multi-selective inhibitor has a K_{D}1 value of less than 1 nM. In some embodiments, the RAS(ON) multi-selective inhibitor has a K_{D}1 value of less than 10 nM. In some embodiments, the RAS(ON) multi-selective inhibitor has a K_{D}1 value of less than 50 nM. In some embodiments, the RAS(ON) multi-selective inhibitor has a K_{D}1 value of less than 100 nM. In some embodiments, the RAS(ON) multi-selective inhibitor has a K_{D}1 value of less than 250 nM. In some embodiments, the RAS(ON) multi-selective inhibitor has a K_{D}1 value of less than 500 nM.

In some embodiments, the RAS(ON) multi-selective inhibitor has a clearance rate of 0.4 L/h/kg or slower (e.g., 0.4 L/h/kg, 0.3 L/h/kg, 0.2 L/h/kg, 0.1 L/h/kg, 0.05 L/h/kg, 0.025 L/h/kg, 0.0125 L/h/kg, or 0.006 L/h/kg). In some embodiments, the clearance rate is determined in a human subject. In some embodiments, the clearance rate is from noncompartmental analysis using measured inhibitor concentrations over time in one or more blood samples. In some embodiments, the clearance rate is determined in blood from preclinical species and allometrically scaled to human. In some embodiments, the clearance rate is determined by applying PK simulation and modeling approaches to estimate clearance based on in silico human physiological conditions.

In some embodiments, the intermittent dosing regimen includes one dosing day followed by at least one day without dosing.

In some embodiments, the intermittent dosing regimen includes at least two consecutive dosing days followed by at least one day without dosing.

In some embodiments, the intermittent dosing regimen includes at least three consecutive dosing days followed by at least one day without dosing.

In some embodiments, the intermittent dosing regimen includes at least four consecutive dosing days followed by at least one day without dosing.

In some embodiments, the intermittent dosing regimen includes at least five consecutive dosing days followed by at least one day without dosing.

In some embodiments, the intermittent dosing regimen includes at least six consecutive dosing days followed by at least one day without dosing.

In some embodiments, each dosing regimen includes five dosing days and two days without dosing.

In some embodiments, each dosing regimen includes four dosing days and three days without dosing.

In some embodiments, each dosing regimen includes three dosing days and four days without dosing.

In some embodiments, each dosing regimen includes two dosing days and five days without dosing.

In some embodiments, each dosing regimen includes one dosing day and six days without dosing.

In some embodiments, each dosing regimen includes seven consecutive days of dosing (e.g., Q2W).

In some embodiments, the RAS(ON) multi-selective inhibitor is administered Q2D.

In some embodiments, the intermittent dosing regimen is repeated.

In some embodiments of the intermittent dosing methods described herein includes administering a dose of 0.001 mg to 2000 mg per day, for example, 10 mg to 1000 mg (e.g., 10 mg, 20 mg, 30 mg, 40 mg, 50, mg 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg) of a RAS(ON) multi-selective inhibitor, as disclosed herein, to the subject on a dosing day.

In some embodiments, the daily dose of the RAS(ON) multi-selective inhibitor is administered once per day.

In some embodiments, the daily dose of the RAS(ON) multi-selective inhibitor is divided equally into twice per day.

In some embodiments, the dosing regimen includes administering a first RAS(ON) multi-selective inhibitor and an additional therapeutic agent. In some embodiments, the additional therapeutic agent is a RAS(OFF) inhibitor. In some embodiments, the additional therapeutic agent is a pan-KRAS inhibitor. In some embodiments, the pan-KRAS inhibitor is ERAS-4001.

In some embodiments, the dosing regimen includes administering a first RAS(ON) multi-selective inhibitor and a second RAS inhibitor.

In some embodiments, the first RAS(ON) multi-selective inhibitor and the second RAS inhibitor are not identical.

In some embodiments, the RAS(ON) multi-selective inhibitor is

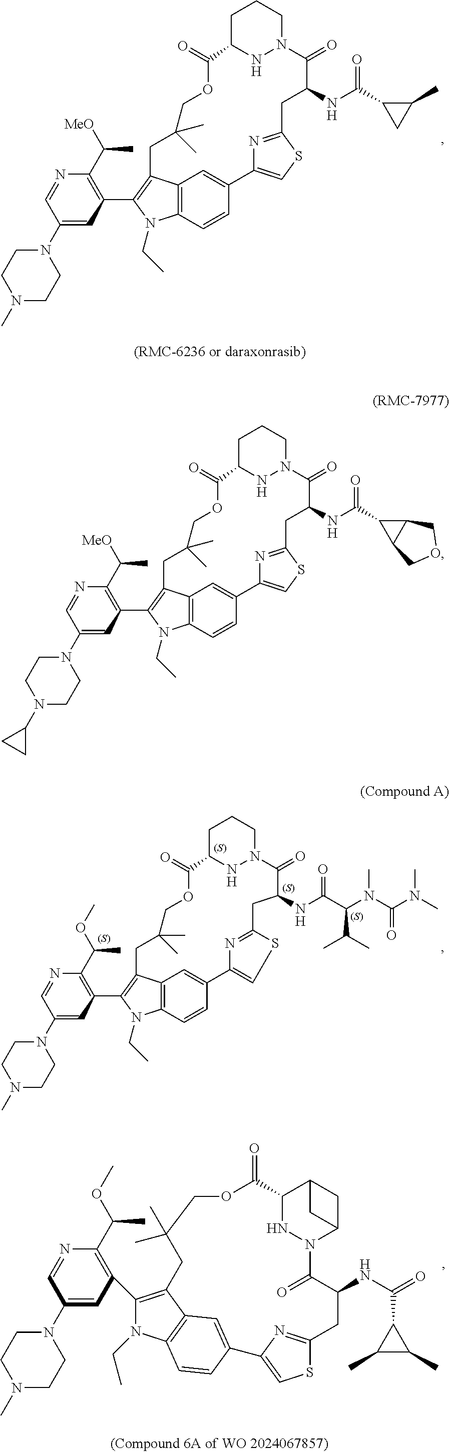

### or ERAS-0015.

In some embodiments, the second RAS inhibitor is a pan-KRAS inhibitor.

In some embodiments, the pan-KRAS inhibitor is ERAS-4001.

In another aspect, the invention features a method of treating a RAS protein-related disease or disorder, including administering to a subject in need thereof a RAS(ON) multi-selective inhibitor and an additional therapeutic agent, wherein the RAS(ON) multi-selective inhibitor is administered on an intermittent regimen.

In some embodiments, the additional therapeutic agent is an additional RAS inhibitor and the RAS inhibitor is administered on a daily dosing regimen (i.e., QD) or on an intermittent dosing regimen.

In some embodiments, the additional RAS inhibitor is a RAS(OFF) inhibitor.

In some embodiments, the additional RAS inhibitor is a pan-KRAS inhibitor.

In some embodiments, the pan-KRAS inhibitor is ERAS-4001.

In some embodiments, the RAS(ON) multi-selective inhibitor is ERAS-0015 and the additional RAS inhibitor is ERAS-4001. In some embodiments, the RAS(ON) multi-selective inhibitor is ERAS-0015 and the additional therapeutic agent is an anti-PD1 inhibitor (e.g., pembrolizumab, BNT327, and ivonescimab). In some embodiments, the RAS(ON) multi-selective inhibitor is ERAS-0015 and the additional therapeutic agent is an anti-EGFR inhibitor (e.g., panitumumab).

In some embodiments, the subject has a RAS mutation. In some embodiment, the RAS mutation is a KRAS mutation.

In some embodiments, the RAS(ON) multi-selective inhibitor and the additional RAS inhibitor are not identical.

In some embodiments, the RAS(ON) multi-selective inhibitor is administered on the first, third, and fifth day of the intermittent dosing regimen and not administered on the second, fourth and sixth day of the intermittent dosing regimen.

In some embodiments, the additional RAS inhibitor is administered on the second, fourth and sixth day and not administered on the third, fifth, and seventh day of the intermittent dosing regimen.

In some embodiments, the RAS(ON) multi-selective inhibitor has a blood or plasma half-life of 12 hours or longer (e.g., 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, or 50 hours).

In some embodiments, the RAS(ON) multi-selective inhibitor has a K_{D}1 of 0.1 nM to 500 nM. In some embodiments, the RAS(ON) multi-selective inhibitor has a K_{D}1 value of less than 1 nM. In some embodiments, the RAS(ON) multi-selective inhibitor has a K_{D}1 value of less than 10 nM. In some embodiments, the RAS(ON) multi-selective inhibitor has a K_{D}1 value of less than 50 nM. In some embodiments, the RAS(ON) multi-selective inhibitor has a K_{D}1 value of less than 100 nM. In some embodiments, the RAS(ON) multi-selective inhibitor has a K_{D}1 value of less than 250 nM. In some embodiments, the RAS(ON) multi-selective inhibitor has a K_{D}1 value of less than 500 nM.

In some embodiments, the RAS(ON) multi-selective inhibitor has a clearance rate of 0.4 L/h/kg or slower (e.g., 0.4 L/h/kg, 0.35 L/h/kg, 0.3 L/h/kg, 0.25 L/h/kg, 0.2 L/h/kg, 0.1 L/h/kg, 0.05 L/h/kg, 0.03 L/h/kg, 0.01 L/h/kg, or 0.006 L/h/kg). In some embodiments, the clearance rate is determined in a human subject. In some embodiments, the clearance rate is from noncompartmental analysis using measured inhibitor concentrations over time in one or more blood samples. In some embodiments, the clearance rate is determined in blood from preclinical species and allometrically scaled to human. In some embodiments, the clearance rate is determined by applying PK simulation and modeling approaches to estimate clearance based on in silico human physiological conditions.

In some embodiments, the intermittent dosing regimen includes one dosing day followed by at least one day without dosing.

In some embodiments, the intermittent dosing regimen includes at least two consecutive dosing days followed by at least one day without dosing.

In some embodiments, the intermittent dosing regimen includes at least three consecutive dosing days followed by at least one day without dosing.

In some embodiments, the intermittent dosing regimen includes at least four consecutive dosing days followed by at least one day without dosing.

In some embodiments, the intermittent dosing regimen includes at least five consecutive dosing days followed by at least one day without dosing.

In some embodiments, the intermittent dosing regimen includes at least six consecutive dosing days followed by at least one day without dosing.

In some embodiments, each dosing regimen includes five dosing days and two days without dosing.

In some embodiments, each dosing regimen includes four dosing days and three days without dosing.

In some embodiments, each dosing regimen includes three dosing days and four days without dosing.

In some embodiments, each dosing regimen includes two dosing days and five days without dosing.

In some embodiments, each dosing regimen includes one dosing day and six days without dosing.

In some embodiments, each dosing regimen includes seven consecutive days of dosing (e.g., Q2W).

In some embodiments, the RAS(ON) multi-selective inhibitor is administered Q2D.

In some embodiments, the intermittent dosing regimen is repeated.

In some embodiments of the intermittent dosing methods described herein includes administering a dose of 0.001 mg to 2000 mg per day, for example, 10 mg to 1000 mg (e.g., 10 mg, 20 mg, 30 mg, 40 mg, 50, mg 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg) of a RAS(ON) multi-selective inhibitor, as disclosed herein, to the subject on a dosing day.

In some embodiments, the daily dose of the RAS(ON) multi-selective inhibitor is administered once per day.

In some embodiments, the daily dose of the RAS(ON) multi-selective inhibitor is divided equally into twice per day.

In some embodiments, the RAS(ON) multi-selective inhibitor is RMC-6236 (daraxonrasib), RMC-7977, Compound A, (Compound 6A of WO 2024067857), or ERAS-0015.

### BRIEF DESCRIPTION OF THE FIGURES

FIG. 1 graphically depicts the anti-tumor effect by RMC-7977, RMC-6236 (daraxonrasib), and Compound A in NCI-H441, KRAS^{G12V} non-small cell lung cancer (NSCLC) xenograft model.

FIG. 2 shows the PD effects by RMC-7977, RMC-6236 (daraxonrasib), and Compound A in NCI-H441, KRAS^{G12V} NSCLC xenograft model.

FIG. 3 shows antitumor effect and tolerability of Compound A.

FIG. 4 shows antitumor effect and tolerability of RMC-6236 (daraxonrasib).

FIG. 5 shows antitumor effect and tolerability of higher doses of RMC-6236 (daraxonrasib).

FIG. 6 shows intermittent dosing of RMC-6236 (daraxonrasib) allows for increased dose intensity in the NCI-H441 xenograft model.

FIG. 7 shows antitumor activity and tolerability of various dosing schedules of RMC-6236 (daraxonrasib) in the Capan-2, KRAS^{G12V} PDAC CDX model.

### DETAILED DESCRIPTION

The present disclosure is based, in part, on the discovery that intermittent administration of RAS(ON) multi-selective inhibitors is an effective therapeutic method for treating RAS protein-related disease. RAS(ON) multi-selective inhibitors bind to chaperone protein cyclophilin A (CypA) to form a binary complex, that then inhibits the RAS proteins (e.g., mutant and wild-type RAS variants) by forming a tri-complex structure, see e.g., Holderfield et al., Nature, volume 629, pages 919-926 (2024), Cregg et al., Journal of Medicinal Chemistry, volume 68, issue 6 (2025), and Jiang et al., Cancer Discovery, 14(6) (2024). Due to their ability to inhibit both mutant and wild-type RAS isoforms (e.g., KRAS, NRAS, and HRAS), RAS(ON) multi-selective inhibitors offer broad therapeutic potential across RAS protein-related diseases. However, some have feared that inhibition of wild-type RAS would produce severe, dose-limiting toxicities, based on genetic studies demonstrating RAS's essential role in embryonic development and in normal hematopoiesis.

Clinical data from the Phase I study of RMC-6236 (daraxonrasib) (NCT05379985), a RAS(ON) multi-selective inhibitor, have demonstrated an acceptable safety profile and encouraging antitumor activity in patients with RAS mutant NSCLC or PDAC. The most common treatment-related adverse events (TRAEs) observed were rash and GI-related toxicities, which are consistent with the known on-target toxicities in normal tissues reported from other RAS pathway inhibitors. These data supported the initiation of Phase 3 studies of RMC-6236 in patients with PDAC (NCT06625320) or NSCLC (NCT06881784).

Binding affinity of a RAS(ON) inhibitor to CypA is determined by K_{D}1, and without being bound by theory, the inventors/applicant believe that RAS(ON) multi-selective compounds with a higher binding affinity (i.e., low K_{D}1 value) to CypA tend to have higher exposure and longer half-life in tissues. This can result in prolonged RAS pathway inhibition in normal (non-tumor) tissues, which can elicit toxicities that can be dose-limiting (such as rash and GI toxicities resulting from RAS pathway inhibition in those tissues).

Thus, for those compounds with low K_{D}1 values, an intermittent dosing schedule is considered to allow a sufficiently long dosing interval, so that the drug concentrations in normal tissues could be decreased to a certain concentration that allows RAS pathway reactivation, and thus minimize toxicity to normal tissues. Accordingly, the present disclosure provides methods of administering RAS(ON) multi-selective inhibitors, particularly those having higher binding affinity to CypA (i.e., low K_{D}1 value), using intermittent dosing regimens. The present disclosure also provides methods of selecting or identifying RAS(ON) multi-selective inhibitors to be administered on an intermittent dosing regimen.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Note that when a range or amount is provided in the disclosure herein, 5% of each range endpoint or specific amount is included, unless otherwise indicated. For example, a range of 200 mg to 1000 mg of a RAS(ON) multi-selective inhibitor is understood to encompass 200±5% mg to 1000±5% mg, e.g., 190 mg to 1050 mg of a RAS(ON) multi-selective inhibitor.

As used herein, the term “administration” refers to the administration of a composition comprising a RAS(ON) inhibitor compound to a subject or system. Administration also includes administering a prodrug derivative or analog or pharmaceutically acceptable salt to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal. In some embodiments, a composition comprising the RAS(ON) inhibitor compound is administered orally.

Unless otherwise indicated, the term “at least” preceding a series of elements is to be understood to refer to every element in the series and any one or any and all combinations of the elements.

The term “combination therapy” refers to a method of treatment including administering to a subject at least two active therapeutic agents (e.g., a RAS(ON) multi-selective inhibitor and a pan-KRAS inhibitor), optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

As used herein, the term “dosage form” refers to a physically discrete unit of a compound (e.g., the RAS(ON) inhibitor compound) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., the RAS(ON) multi-selective inhibitor compound) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen or “therapy”).

The term “disorder” is used in this disclosure to mean, and is used interchangeably with, the terms disease, condition, or illness, unless otherwise indicated.

The terms “inhibit,” “block,” and “suppress” are used interchangeably and refer to any statistically significant decrease in a biological activity, including full blocking of the activity. As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein.

In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded protein.

A “patient” or “subject” is a mammal, for whom diagnosis, prognosis, or therapy is desired. Mammalian subjects include, but are not limited to, humans, domestic animals, farm animals, sports animals, and zoo animals including, for example, humans, non-human primates, dogs, cats, guinea pigs, rabbits, rats, mice, horses, and cattle. In certain embodiments, the subject has been diagnosed with cancer. In certain embodiments, the subject is a human afflicted with a tumor (e.g., cancer) who has been diagnosed with a need for treatment for a tumor (e.g., cancer).

As used herein, the term “pharmaceutical composition” refers to a compound, such as a RAS(ON) inhibitor compound disclosed herein, or a pharmaceutically acceptable salt thereof, formulated together with a pharmaceutically acceptable excipient.

A “pharmaceutically acceptable excipient,” as used herein, refers to any inactive ingredient (for example, a vehicle capable of suspending or dissolving the active compound) having the properties of being nontoxic and noninflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:119, 1977 and in Pharmaceutical Salts: Properties, Selection, and Use, (Eds. P. H. Stahl and C. G. Wermuth), WileyVCH, 2008. The salts can be prepared in situ during the final isolation and purification of the compounds described herein or separately by reacting the free base group with a suitable organic acid.

The terms “RAS pathway” and “RAS/MAPK pathway” are used interchangeably herein to refer to a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. For example, SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The terms “RAS inhibitor” and “inhibitor of [a] RAS” are used interchangeably to refer to any inhibitor that targets, that is, selectively binds to or inhibits a RAS protein. A RAS inhibitor may be RO7673396, for example.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). In certain embodiments, a RAS(ON) inhibitor useful in the present disclosure may form a high affinity three-component complex, or conjugate, between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the inhibitors of RAS described herein induce a new binding pocket in RAS by driving formation of a high affinity tri-complex, or conjugate, between the RAS protein and the widely expressed cytosolic chaperone, cyclophilin A (CypA). A RAS(ON) inhibitor may be an antibody-drug conjugate. See also doi.org/10.1021/acs.jmedchem.4c02929.

As used herein, the term “RAS(OFF) inhibitor” refers to an inhibitor that targets, that is, selectively binds to or inhibits, the GDP-bound, inactive state of RAS (e.g., selective over the GTP-bound, active state of RAS). RAS(OFF) inhibitors are known in the art and described. Non-limiting examples of RAS(OFF) inhibitors include A2A-03, ABREV01, ABT-200, ADT-030, ADT-1004, AN9025, BBP-454, BGB-53038, BI-2865, BI-2493, BI 3706674, ERAS-4, ERAS-254, ERAS-4001, HB-700 (G12X+G13D), JAB-23400, OC211, PF-07934040, QTX3034, RSC-1255, YL-17231, ZG2001, PF-07985045, ADT-007, SIL204, and HZ-V068. Non-limiting examples of RASG12C(OFF) inhibitors include adagrasib (MRTX849), divarasib (RG6330/GDC-6036), fulzerasib (IB1351/GFH925), garsorasib (D-1553), glecirasib (JAB-21822), olomorasib (LY3537982), opnurasib (JDQ443), sotorasib (AMG 510), ARS-853, ARS-1620, BI 1823911, BPI-421286, D3S-001, GEC255, HBI-2438, HS-10370, JAB-21000, JAB-21822, JMKX001899, JNJ-74699157 (ARS-3248), MK-1084, YL-15293, SK-17, and BI-0474. Non-limiting examples of RASG12D(OFF) inhibitors include ASP3082, BPI-501836, ERAS-4693, ERAS-5024, HBW-012-D, HBW-012-E, HBW-012336, HRS-4642, JAB-22000, KD-8, TSN1611, LY3962673, MRTX282, MRTX1133, Q2a, SHR1127, TH-Z827, TH-Z835, TSN1611, VRTX153, DN022150, GDC-7035, AZD0022, RNK08954, INCB186748, AST2169, and QLC1101. Non-limiting examples of RASG12V(OFF) inhibitors include JAB-23000 and QTX3544.

As used herein, the terms “RAS(ON) multi-selective inhibitor,” “RASMULTI inhibitor,” “RASMULTI(ON) inhibitor,” and “RAS(MULTI) inhibitor” refer to a RAS inhibitor of at least three RAS isoforms, including wild-type and/or variants with missense mutations at one of the following positions: 12, 13, 59, 61, or 146. In some embodiments, a RAS(ON) multi-selective inhibitor (e.g., daraxonrasib or RMC-6236) refers to a RAS inhibitor of at least three RAS variants with missense mutations at one of the following positions: 12, 13, and 61. Exemplary RAS(ON) multi-selective inhibitors include but are not limited to compounds described in the following patent applications, and as otherwise described herein: WO 2025087431, WO 2025051241, WO 2025045233, WO 2024249299, WO 2024222864, WO 2024206858, WO 2024169914, WO 2024153208, WO 2024149214, WO 2024104364, WO 202′4067857, WO 2024060966, WO 2024017859, WO 2024008834, WO 2023240263, WO 2023025832, WO 2022060836, WO 2021091956, CN119350371, CN 117903169, CN 117720556, CN 117720555, CN 117720554, CN 117534687, CN 117534685, and CN 117534684, each of which is incorporated herein by reference in its entirety, including the compound structures disclosed therein. Non-limiting examples of RAS(ON) multi-selective inhibitors also include daraxonrasib (RMC-6236), RMC-7977, Compound A, GFH547, ERAS-0015, BI-2852, BPI-572270, RCZY-690, RCZY-680 and compound 6A of WO 2024067857.

As used herein, the terms “RAS(ON) mutant-selective inhibitor” refers to a RAS inhibitor selective for a RAS(ON) variant with missense mutation at one of the following positions: 12, 13, or 61. Non-limiting examples of RAS(ON) mutant-selective inhibitors include RAS(ON) G12C-selective inhibitors (e.g., elironrasib or RMC-6291), RAS(ON) G12D-selective inhibitors (e.g., zoldonrasib or RMC-9805), RAS(ON) Q61H-selective inhibitors (e.g., RMC-0708), RAS(ON) G12V-selective inhibitors (e.g. RMC-5127), and RAS(ON) G13D-selective inhibitors. RAS(ON) mutant-selective inhibitors can be found in any one of the following patent applications, and as otherwise described herein: WO 2025104149, WO 2025093625, WO 2025080946, WO 2024249299, WO 2024211663, WO 2024211712, WO 2024208934, WO 2024149819, WO 2024008610, WO 2024102421, WO 2023240263, WO 2023133543, WO 2023015559, WO 2023086341, WO 2023208005, WO 2023232776, WO 2023086341, WO 2023060253, WO 2023015559, WO 2022235870, WO 2022235864, WO 2021091967, WO 2021091982, WO 2021108683, WO 2020132597, each of which is incorporated herein by reference in its entirety, including the compound structures disclosed therein.

A “therapeutic agent” is any substance, e.g., a compound or composition, capable of treating a disease or disorder. In some embodiments, therapeutic agents that are useful in connection with the present disclosure including RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “treatment” (also “treat” or “treating”), in its broadest sense, refers to any administration of a substance (e.g., a RAS(ON) multi-selective inhibitor compound) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who is diagnosed with the disease, disorder or condition but does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In any treatment method herein, a patient or subject may be in need of such treatment.

The term “wild type” refers to an entity having a structure or activity as found in nature in a “normal” (as contrasted with mutant, diseased, altered, etc.) state or context. Those of ordinary skill in the art will appreciate that wild type genes and polypeptides often exist in multiple different forms (e.g., alleles).

### Treatment Methods

The present disclosure provides, inter alia, the use of a RAS(ON) multi-selective inhibitors in methods of treating subjects with a RAS protein-related disease through administering the RAS(ON) multi-selective inhibitor on an intermittent dosing regimen as disclosed herein. The disclosure also provides for methods of selecting or identifying a RAS(ON) multi-selective inhibitor for use in the intermittent dosing regimens of the disclosure. In general, the disclosure features methods of treating a RAS protein-related disease (e.g., RAS mutant cancer) in a human subject in need thereof, the methods comprise intermittent administration of an effective amount of a RAS(ON) multi-selective inhibitor to the subject, effective to treat the RAS protein-related disease. RAS(ON) multi-selective inhibitors disclosed herein may be administered or formulated in combination with an additional therapeutic agent also described herein.

In some embodiments, a RAS(ON) multi-selective inhibitor useful in the methods according to the present disclosure has a low K_{D}1 value (i.e., high binding affinity to CypA). In certain embodiments, the RAS(ON) multi-selective inhibitor has a K_{D}1 value of 0.1 nM to 500 nM. In certain embodiments, the RAS(ON) multi-selective inhibitor has a K_{D}1 value of less than 0.1 nM. In certain embodiments, the RAS(ON) multi-selective inhibitor has a K_{D}1 value of less than 1 nM. In certain embodiments, the RAS(ON) multi-selective inhibitor has a K_{D}1 value of less than 10 nM. In certain embodiments, the RAS(ON) multi-selective inhibitor has a K_{D}1 value of less than 50 nM. In certain embodiments, the RAS(ON) multi-selective inhibitor has a K_{D}1 value of less than 100 nM. In certain embodiments, the RAS(ON) multi-selective inhibitor has a K_{D}1 value of less than 250 nM. In certain embodiments, the RAS(ON) multi-selective inhibitor has a K_{D}1 value of less than 500 nM.

In some embodiments, a RAS(ON) multi-selective inhibitor has a K_{D}1 value of 0.01 nM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 15 nM, 16 nM, 17 nM, 18 nM, 9 nM, 0 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM, 151 nM, 152 nM, 153 nM, 154 nM, 155 nM, 156 nM, 157 nM, 158 nM, 159 nM, 160 nM, 161 nM, 162 nM, 163 nM, 164 nM, 165 nM, 166 nM, 167 nM, 168 nM, 169 nM, 170 nM, 171 nM, 172 nM, 173 nM, 174 nM, 175 nM, 176 nM, 177 nM, 178 nM, 179 nM, 180 nM, 181 nM, 182 nM, 183 nM, 184 nM, 185 nM, 186 nM, 187 nM, 188 nM, 189 nM, 190 nM, 191 nM, 192 nM, 193 nM, 194 nM, 195 nM, 196 nM, 197 nM, 198 nM, 199 nM, 200 nm, 205 nM, 210 nM, 215 nM, 220 nM, 225 nM, 230 nM, 235 nM, 240 nM, 245 nM, 250 nM, 255 nM, 260 nM, 265 nM, 270 nM, 275 nM, 280 nM, 285 nM, 290 nM, 295 nM, 300 nM, 305 nM, 310 nM, 315 nM, 320 nM, 325 nM, 330 nM, 335 nM, 340 nM, 345 nM, 350 nM, 355 nM, 360 nM, 365 nM, 370 nM, 375 nM, 380 nM, 385 nM, 390 nM, 395 nM, 400 nM, 405 nM, 410 nM, 415 nM, 420 nM, 425 nM, 430 nM, 435 nM, 440 nM, 445 nM, 450 nM, 455 nM, 460 nM, 465 nM, 470 nM, 475 nM, 480 nM, 485 nM, 490 nM, 495 nM, or 500 nM.

The K_{D}1 value of a RAS(ON) multi-selective inhibitor can be determined using methods standard in the art, including but not limited to surface plasmon resonance (SPR), Fluorescence Polarization (FP), or isothermal titration calorimetry (ITC). In a non-limiting example, the binding affinity of compounds for CypA can be assessed by SPR using, for example, a Biacore 8K instrument. CypA is immobilized on a sensor chip (e.g., a streptavidin chip), and varying RAS(ON) multi-selective inhibitor compound concentrations can be flowed over the chip in assay buffer. The SPR sensorgrams can be fitted using either a steady state affinity model or a 1:1 binding (kinetic) model to assess the K_{D}1 for CypA binding. In a specific embodiment, the K_{D}1 of a RAS(ON) multi-selective inhibitor is determined using SPR.

The binding affinity of RAS(ON) multi-selective inhibitor compounds for CypA can also be assessed by FP competition with fluorescein tagged cyclosporine A by varying RAS(ON) multi-selective inhibitor compound concentrations. The signals can be fitted using a concentration response curve and the K_{D}1 is calculated.

In some embodiments, the K_{D}1 value of a RAS(ON) multi-selective inhibitor for CypA can be determined using ITC, a label-free biophysical technique that directly measures the heat released or absorbed during molecular interactions. In a non-limiting example, purified human CypA protein is prepared in an appropriate buffer (e.g., phosphate-buffered saline or HEPES) and loaded into the sample cell of an ITC instrument (e.g., MicroCal PEAQ-ITC or equivalent). A solution of the RAS(ON) multi-selective inhibitor is prepared at a higher concentration and titrated stepwise into the sample cell under isothermal conditions. As the inhibitor binds to CypA, the heat of binding is measured after each injection. The resulting thermogram (a plot of heat change vs. time) is integrated and fit to a binding model (e.g., one-site model) to derive binding thermodynamics, including the equilibrium dissociation constant (KD), stoichiometry (n), enthalpy (ΔH), and entropy (ΔS) of binding.

The amino acid sequence of human CypA suitable for use in the binding assays described herein is known in the art and can be found, for example, at NCBI Reference Sequence NP_066953.1.

In some embodiments, a RAS(ON) multi-selective inhibitor useful for administration in an intermittent dosing regimen according to the present disclosure exhibits retention in blood and/or tissue, as measured by its blood or tissue half-life. Blood (e.g., plasma) half-life refers to the time required for the concentration of a RAS(ON) multi-selective inhibitor compound in the blood plasma to reduce to half its initial value. Tissue half-life refers to the time required for the concentration of a RAS(ON) multi-selective inhibitor compound in the tissue to reduce to half its initial value. In embodiments, the RAS(ON) multi-selective inhibitor has a blood or tissue half-life of 6 hours or more (e.g., a half-life of 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hour, 27 hours, 28 hours, 29 hours, 30 hours, 31 hours, 32 hours, 33, hours 34, hours, 35 hours, 36 hours, 37 hours, 38 hours, 39 hours, 40 hours or longer). The blood or tissue half-life of a RAS(ON) multi-selective inhibitor can be determined using noncompartmental analysis methods known in various pharmacokinetic (PK) software (e.g., Phoenix WinNonlin). Such means include, but are not limited to, measuring the concentration of the RAS(ON) multi-selective inhibitor in blood or tissue samples obtained over time using appropriate analytical techniques such as high-performance liquid chromatography (HPLC), mass spectrometry (MS), LC-MS/MS or enzyme-linked immunosorbent assay (ELISA).

In some embodiments, a RAS(ON) multi-selective inhibitor useful for an intermittent dosing administration regimen according to the present disclosure has a blood clearance rate of 0.4 L/h/kg or slower. Clearance rate, also known as the rate of elimination, refers to the rate at which a substance (e.g., a RAS(ON) multi-selective inhibitor) is removed from the body, typically the blood, per unit of time. For small molecules, this is often measured in units of volume per unit time, like milliliters per minute (mL/min) or liters per hour (L/h). It indicates how quickly a drug or substance is being removed from circulation. The clearance rate may be determined in a human subject or clearance studies can be conducted in suitable species and modeling used to calculate clearance in a human subject. In some embodiments, the clearance rate can be determined using noncompartmental analysis methods known in various pharmacokinetics software. Such means include, but are not limited to, determining the clearance of the RAS(ON) multi-selective inhibitor in blood from noncompartmental analysis using measured inhibitor concentrations in human blood, determining the clearance of inhibitor in blood from preclinical species and then allometrically scaled to human, measuring the intrinsic clearance from in vitro metabolic assays and extrapolated to in vivo in human; or applying PK simulation and modeling approaches to estimate clearance based on in silico human physiological conditions.

RAS(ON) multi-selective inhibitors having one or more of these properties, and thus useful in the methods according to the disclosure, can be found in any one of the following patent applications: WO 2025087431, WO 2025051241, WO 2025045233, WO 2024249299, WO 2024222864, WO 2024206858, WO 2024169914, WO 2024153208, WO 2024149214, WO 2024104364, WO 2024067857, WO 2024060966, WO 2024017859, WO 2024008834, WO 2023240263, WO 2023025832, WO 2022060836, WO 2021091956, CN119350371, CN 117903169, CN 117720556, CN 117720555, CN 117720554, CN 117534687, CN 117534685, and CN 117534684, each of which is incorporated herein by reference in its entirety, including the compound structures disclosed therein.

The RAS(ON) multi-selective compounds useful according to the present disclosure exhibit inhibitory activities across a variety of RAS mutants. In some embodiments, a RAS(ON) multi-selective compound inhibits wild type RAS. In some embodiments, a RAS(ON) multi-selective compound inhibits wild type KRAS. In some embodiments, a RAS(ON) multi-selective compound inhibits a RAS mutant with one or more mutations at G12X, G13X, and/or Q61X, wherein X represents any naturally occurring amino acid residue. In certain instances, X is A, C, D, V, S, R, H, K, or L amino acid residue.

In certain embodiments, a RAS(ON) multi-selective compound inhibits a RAS mutant with one or more mutations at G12X, wherein X represents any naturally occurring amino acid residue. In certain instances, X is A, C, D, V, S or R amino acid residue.

In other embodiments, a RAS(ON) multi-selective compound inhibits a RAS mutant with one or more mutations at G13X, wherein X is any naturally occurring amino acid residue. In certain instances, X is A, C, D, V, S or R amino acid residue.

In other embodiments, a RAS(ON) multi-selective compound inhibits a RAS mutant with one or more mutations at Q61X, wherein X is any naturally occurring amino acid residue. In certain instances, X is A, C, D, V, S, R, H, K, or L amino acid residue. In other instances, X is H, K, R, or L amino acid residue.

A variety of RAS proteins may be inhibited by a RAS(ON) multi-selective compound (e.g., KRAS, NRAS, HRAS, and mutants thereof at positions 12, 13 and 61, such as G12A, G12C, G12D, G12V, G12S, G12R, G13C, G13D, Q61H, Q61K, Q61R and Q61L, and others described herein, or a combination thereof). In some embodiments, a RAS(ON) multi-selective compound inhibits a G12A, G12C, G12D, G12R, G12S, G12V, or Q61H mutant of RAS, or a combination thereof.

The RAS(ON) inhibitor compounds described herein may be made from commercially available starting materials or synthesized using known organic, inorganic, or enzymatic processes. By way of non-limiting example, the RAS(ON) compounds can be synthesized using the methods described in WO 2022060836, WO 2021091956, or WO 2021091982, together with synthetic methods known in the art of synthetic organic chemistry, or variations thereon as appreciated by those skilled in the art.

RAS(ON) multi-selective inhibitor structural motifs for modulating binding affinity to CypA have been discussed in the art, for example, Holderfield et al., Nature, volume 629, pages 919-926 (2024) and Cregg et al., Journal of Medicinal Chemistry, volume 68, issue 6 (2025), each of which are incorporated by reference in their entirety.

In one aspect, the present disclosure provides methods of selecting or identifying a RAS(ON) multi-selective inhibitor for an intermittent dosing regimen, the methods generally comprise determining the K_{D}1 of the RAS(ON) multi-selective inhibitor, and selecting or identifying the RAS(ON) multi-selective inhibitor as suitable for an intermittent dosing regimen when the RAS(ON) multi-selective inhibitor has a K_{D}1 value of less than 500 nM, less than 250 nM, less than 100 nM, less than 50 nM, less than 10 nM, less than 1 nM or less than 0.1 nM. In some embodiments, the K_{D}1 value is determined using SPR, FP, or ITC. In some embodiments, the methods further include determining one or more additional properties such as clearance rates, blood half-life, and tissue half-life of the RAS(ON) multi-selective inhibitor.

In some embodiments, the RAS(ON) multi-selective inhibitor useful for an intermittent dosing administration regimen according to the present disclosure is RMC-6236 (daraxonrasib)

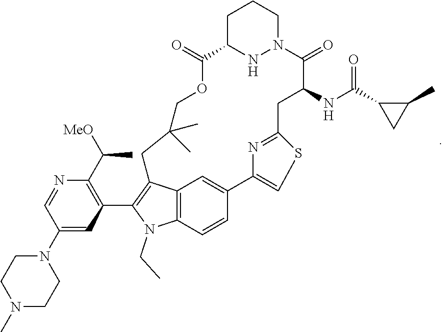

In some embodiments, the RAS(ON) multi-selective inhibitor is RMC-7977

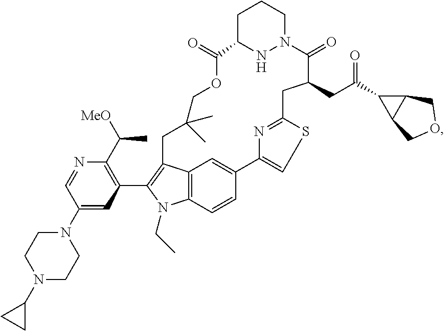

In some embodiments, the RAS(ON) multi-selective inhibitor is Compound A

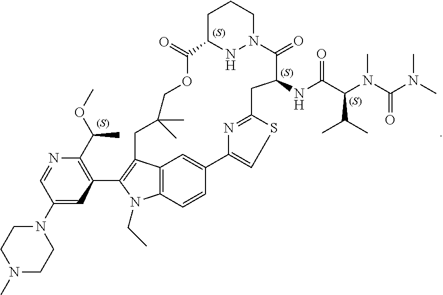

In some embodiments, the RAS(ON) multi-selective inhibitor is:

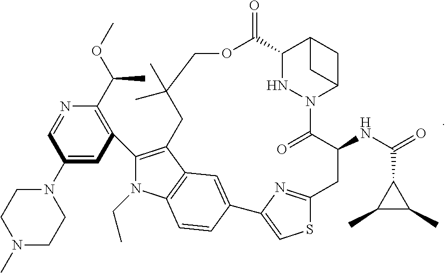

In some embodiments, the RAS(ON) multi-selective inhibitor is ERAS-0015. Erasca has publicly disclosed data on ERAS-0015, including information presented in corporate materials, which show that ERAS-0015 binds CypA with high affinity, exhibiting a K_{D}1 value of approximately 4.5 nM by surface plasmon resonance and 5.3 nM by isothermal titration calorimetry. These K_{D}1 values fall within the high affinity CypA binders specified in the present disclosure. Accordingly, ERAS-0015 is representative of RAS(ON) multi-selective inhibitors that may benefit from administration using intermittent dosing regimens as disclosed herein. Furthermore, ERAS-0015 has been reported to exhibit prolonged tissue retention (e.g., an estimated half-life of 24 hours or more and a clearance rate slower than 0.4 L/h/kg), consistent with the characteristics identified useful for intermittent dosing herein and thereby reducing wild-type RAS inhibition and related toxicities in normal tissues.

Intermittent administration is administration of a pharmaceutical composition (e.g., comprising a RAS(ON) multi-selective inhibitor compound) at times that are more than one day apart. In a non-limiting example, an intermittent dosing regimen includes at least one administration day and at least one day without administration. In embodiments, the times between administrations may be two days, several days, one week, several weeks, one month, several months, or may be longer. The times between administrations may be regular (e.g., the time between administrations is always the same number of days), or may be irregular e.g., the time between some pairs of administrations of the pharmaceutical composition is a different number of days than the time between other pairs of administrations of the pharmaceutical composition). In embodiments, the time between administrations between a first and a second administration of the pharmaceutical composition need not be the same as the time between administrations between a second and a third, or between a third and a fourth administration of the pharmaceutical composition, or between other subsequent administrations of the pharmaceutical composition.

In embodiments of the methods and uses disclosed herein, intermittent administration comprises administration of an effective amount of a RAS(ON) multi-selective inhibitor as disclosed herein on an intermittent dosing regimen where the intermittent dosing regimen comprises one dosing day followed by at least one day without dosing. In certain embodiments, the intermittent dosing regimen comprises at least two consecutive dosing days followed by at least one day without dosing. In certain embodiments, the intermittent dosing regimen comprises at least three consecutive dosing days followed by at least one day without dosing. In certain embodiments, the intermittent dosing regimen comprises at least four consecutive dosing days followed by at least one day without dosing. In certain embodiments, the intermittent dosing regimen comprises at least five consecutive dosing days followed by at least one day without dosing. In certain embodiments, the intermittent dosing regimen comprises at least six consecutive dosing days followed by at least one day without dosing. In certain embodiments, the intermittent dosing regimen comprises five dosing days and two days without dosing. In certain embodiments, the intermittent dosing regimen comprises four dosing days and three days without dosing. In certain embodiments, the intermittent dosing regimen comprises three dosing days and four days without dosing. In certain embodiments, the dosing regimen comprises seven consecutive days of dosing.

In certain embodiments, a RAS(ON) multi-selective inhibitor as disclosed herein is administered Q2D. In each of the preceding embodiments, the methods according to the disclosure include repeating the dosing regimen. In some embodiments, the dosing regimen is repeated weekly (i.e., on a 7 day cycle). In some embodiments, a RAS(ON) multi-selective inhibitor as disclosed herein is administrated according to an intermittent dosing regimen for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 weeks.

In some embodiments of the intermittent dosing methods described herein includes administering a dose of 0.001 mg to 2000 mg per day, for example, 10 mg to 1000 mg (e.g., 10 mg, 20 mg, 30 mg, 40 mg, 50, mg 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or 1000 mg) of a RAS(ON) multi-selective inhibitor, as disclosed herein, to the subject on a dosing day.

In various embodiments, a RAS(ON) multi-selective inhibitor as disclosed herein is administered on dosing days of the intermittent dosing regimen once per day. In some embodiments, a RAS(ON) multi-selective inhibitor as disclosed herein is administered on dosing days of the intermittent dosing regimen is administered once, twice, or more times per day, for example, in a divided daily dose, such as two, three, four, five, or six times a day.

In one aspect, the present disclosure provides methods of reducing RAS inhibition in normal tissues of a subject in need of and receiving a RAS(ON) multi-selective inhibitor, the method comprising administering the RAS(ON) multi-selective inhibitor on an intermittent dosing regimen as disclosed herein. In some embodiments, the RAS(ON) multi-selective inhibitor has a K_{D}1 value less than 500 nM, a blood or tissue half-life of 12 hours or longer, and/or a clearance rate of 0.4 L/h/kg or slower. In some embodiments, the subject is afflicted with a RAS protein-related disease. In some embodiments, the RAS protein-related disease is a RAS mutant cancer.

In another aspect, the present disclosure provides methods of allowing reactivation of the RAS signaling pathway in normal tissues of a subject in need of and receiving a RAS(ON) multi-selective inhibitor, the method comprising administering the RAS(ON) multi-selective inhibitor on an intermittent dosing regimen as disclosed herein. In some embodiments, the RAS(ON) multi-selective inhibitor has a K_{D}1 value less than 500 nM, a blood or tissue half-life of 12 hours or longer, and/or a clearance rate of 0.4 L/h/kg or slower. In some embodiments, the subject is afflicted with a RAS protein-related disease. In some embodiments, the RAS protein-related disease is a RAS mutant cancer.

In yet another aspect, the present disclosure provides methods of reducing RAS(ON) multi-selective inhibitor retention in non-tumor tissues of a subject in need of and receiving a RAS(ON) multi-selective inhibitor, the method comprising administering the RAS(ON) multi-selective inhibitor on an intermittent dosing regimen as disclosed herein. In some embodiments, the RAS(ON) multi-selective inhibitor has a K_{D}1 value less than 500 nM, a blood or tissue half-life of 12 hours or longer, and/or a clearance rate of 0.4 L/h/kg or slower.

In still another aspect, the present disclosure provides methods of minimizing dose-limiting toxicities associated with a RAS(ON) multi-selective inhibitor a subject in need of and receiving a RAS(ON) multi-selective inhibitor, the method comprising administering the RAS(ON) multi-selective inhibitor on an intermittent dosing regimen as disclosed herein. In some embodiments, the RAS(ON) multi-selective inhibitor has a K_{D}1 value less than 500 nM, a blood or tissue half-life of 12 hours or longer, and/or a clearance rate of 0.4 L/h/kg or slower. In some embodiments, the subject is afflicted with a RAS protein-related disease. In some embodiments, the RAS protein-related disease is a RAS mutant cancer. In some embodiments, the dose-limiting toxicities include rash or gastrointestinal toxicities.

In another aspect, the present disclosure provides methods of selecting a dosing regimen for a RAS(ON) multi-selective inhibitor, the method comprising: determining the K_{D}1 of the inhibitor to CypA; and selecting a RAS(ON) multi-selective inhibitor for intermittent administration when the K_{D}1 is less than 500 nM. In some embodiments, the methods further comprise administering the RAS(ON) multi-selective inhibitor using an intermittent dosing regimen.

Response rates or results for subjects administered the RAS(ON) inhibitor therapy in the methods disclosed herein can be measured in various ways, after the subject has been taking the RAS(ON) inhibitor therapy a suitable length of time, as is known to those of skill in the art.

In some embodiments, a RAS(ON) multi-selective inhibitor of the disclosure is administered in treatment regimens. In some embodiments, the treatment regimen is 7 days. In some embodiments, the treatment regimen is 21 days. In various embodiments, the subject undergoes 1, 2, 3, or more treatment regimens. In some embodiments, the subject undergoes at least 3 treatment regimens, at least 5 treatment regimens, at least 8 treatment regimens, at least 10 treatment regimens, or at least 15 treatment regimens.

In various embodiments, the subject is administered a RAS(ON) multi-selective inhibitor of the disclosure for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, 24 months or longer. In various embodiments, the subject is administered a RAS(ON) multi-selective inhibitor of the disclosure for at least 1 month. In various embodiments, the subject is administered a RAS(ON) multi-selective inhibitor of the disclosure for at least 3 months. In various embodiments, the subject is administered a RAS(ON) multi-selective inhibitor of the disclosure for at least 6 months. In various embodiments, the subject is administered a RAS(ON) multi-selective inhibitor of the disclosure for at least 8 months.

In some embodiments, the subject being treated by a RAS(ON) multi-selective inhibitor of the disclosure in the disclosed methods is one who has undergone at least one or more prior systemic cancer therapies (e.g., a RAS(ON) multi-selective inhibitor of the disclosure is a second or third line therapy). In some embodiments, the subject being treated by a RAS(ON) multi-selective inhibitor of the disclosure in the disclosed methods is one who has disease progression following at least one prior systemic cancer therapy (i.e., a RAS(ON) multi-selective inhibitor of the disclosure is a second line therapy). In some embodiments, the subject being treated by a RAS(ON) multi-selective inhibitor of the disclosure in the disclosed methods is one who has disease progression following at least two prior systemic cancer therapies (i.e., a RAS(ON) multi-selective inhibitor of the disclosure is a third line therapy). Prior systemic cancer therapies can be any therapy approved by a regulatory authority (e.g., the FDA or EMA) as treatment given type and stage of cancer. In some cases, the prior systemic cancer therapy is a cancer therapy not yet approved by a regulatory’ authority but undergoing clinical trials. If a subject has had a prior systemic cancer therapy, in some cases, the subject has not undergone any systemic cancer therapy for at least one month, at least two months, at least three months, at least four months, at least five months, or at least six months prior to starting therapy as disclosed herein with a RAS(ON) multi-selective inhibitor of the disclosure.

The subject can respond to the therapy as measured by at least a stable disease (SD), as determined by Response Evaluation Criteria in Solid Tumors (RECIST) 1.1 protocol (Eisenhauer, et al., 2009). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively. The amount of a tumor in an individual is the “tumor burden” which can be measured as the number, volume, and/or weight of the tumor.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

As used herein, “progression free survival” or “PFS” is the time from treatment to the date of the first confirmed disease progression per RECIST 1.1 criteria. In various embodiments, the patient exhibits a PFS of at least 1 month. In various embodiments, the patient exhibits a PFS of at least 3 months. In some embodiments, the patient exhibits a PFS of at least 6 months.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, a Journal of the National Cancer Institute, Vol. 92, No. 3, Feb. 2, 2000, and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

As used herein, “survival” refers to the subject remaining alive, and includes overall survival as well as progression free survival.

As used herein, “reducing the tumor,” means reducing the size, volume, or weight of the tumor, reducing the number of metastases, reducing the size or weight of a metastasis, or combinations thereof.

In certain embodiments, a metastasis is cutaneous or subcutaneous. Thus, in certain embodiments, administration of the immune checkpoint inhibitor reduces the size or volume of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the RAS(ON) multi-selective inhibitor therapy, reduces the weight of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the RAS(ON) multi-selective inhibitor therapy, reduces the size or volume of a metastasis by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the RAS(ON) inhibitor therapy or combination therapy comprising the same, reduces the number of metastases by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99% for example, relative to a control drug in a subject of the same genotype. In certain embodiments, combinations of these effects are achieved.

In some embodiments, a biological sample obtained from the subject is used to determine response to treatment with the RAS(ON) inhibitor therapy. As used herein, the term “biological sample” refers to any sample obtained from a subject. A biological sample can be obtained from a subject prior to or subsequent to a diagnosis, at one or more time points prior to or following treatment or therapy, at one or more time points during which there is no treatment or therapy or can be collected from a healthy subject. The biological sample can be a tissue sample or a fluid sample. In certain embodiments, the biological sample includes a tissue sample, a biopsy sample, a tumor aspirate, a bone marrow aspirate, or a blood sample (or a fraction thereof, such as blood or serum). In certain embodiments, the biological sample includes a tumor cell or cancer cell, for example a circulating tumor cell present in a fluid sample, for example, blood or a fraction thereof. In certain embodiments, the biological sample includes a cell free nucleic acid present in a fluid sample, for example, blood or a fraction thereof. In one embodiment, the biological sample comprises a cell lysate (or lysate fraction) or cell extract; or a solution containing one or more molecules derived from a cell or cellular material (for example a polypeptide or nucleic acid). The cell lysate can include proteins, nuclear and/or mitochondrial fractions. In certain embodiments, the cell lysate includes a cytosolic fraction. In certain embodiments, the cell lysate includes a nuclear/mitochondrial fraction and a cytosolic fraction.

The source of a biological sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The biological sample can contain compounds that are not naturally intermixed with the tissue in nature such as preservatives, anticoagulants, buffers, fixatives, nutrients, antibiotics, or the like. The biological sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. However, other tissue and sample types are amenable for use herein. In one embodiment, the other tissue and sample types can be fresh frozen tissue, wash fluids, or cell pellets, or the like. A biological sample can be a tumor sample, which contains nucleic acid molecules from a tumor or cancer. A biological sample that is a tumor sample can be DNA, for example, genomic DNA, or cDNA derived from RNA. In one embodiment, the tumor nucleic acid sample is purified or isolated (for example, it is removed from its natural state). In one embodiment, the sample is a tissue (for example, a tumor biopsy), a CTC or cell free nucleic acid.

In certain embodiments, a tumor sample is isolated from a human subject. In certain embodiments, the analysis is performed on a tumor biopsy embedded in paraffin wax. In one embodiment, the sample can be a fresh frozen tissue sample. In certain embodiments, the sample is a bodily fluid obtained from the subject. The bodily fluid can be blood or fractions thereof (specifically, serum, plasma, urine, saliva, sputum, or cerebrospinal fluid (CSF). The sample can contain cellular as well as extracellular sources of nucleic acid. The extracellular sources can be cell-free nucleic acids and/or exosomes. The methods described herein, including the RT-PCR methods, are sensitive, precise and have multi-analyte capability for use with paraffin embedded samples. See, for example, Cronin et al., Am. J Pathol. 164(1):35-42 (2004).

Additional means for assessing response are described in detail in the examples below and can generally be applied to the methods disclosed herein.

A subject undergoing a therapy is monitored for adverse events (AE) during the course of the therapy. A treatment related AE is an AE that is related to the treatment drug. A treatment emergent AE is one that a subject develops undergoing the treatment that was not present prior to start of therapy. In some cases, the treatment emergent AE is not or suspected not to be related to the treatment itself. AEs are characterized as one of five grades—grade I is a mild AE; grade 2 is a moderate AE; grade 3 is a severe AE; grade 4 is a life-threatening or disabling AE; and grade 5 is death related to AE. In some cases, the subject does not exhibit any grade 3 AE that is treatment related. In some cases, the subject does not exhibit any grade 3 AE. In some cases, the subject does not exhibit any grade 4 AE that is treatment related. In some cases, the subject does not exhibit any grade 4 AE. In various cases, the subject does not exhibit a grade 3 or grade 4 AE that is treatment related after administration of the RAS(ON) inhibitor therapy for at least one month, or at least three months.

In various cases, the subject being treated with the RAS(ON) inhibitor therapy in the methods disclosed herein, does not exhibit any dose limiting toxicities (DLT) at the dose administered. A DLT is any AE meeting the criteria listed below occurring during the first treatment cycle of the RAS(ON) inhibitor therapy (day 1 through day 21) where relationship to the drug cannot be ruled out.

In various embodiments, the disclosure provides a method of treating a RAS protein-related disease or disorder (e.g., cancer) in a subject in need thereof comprising administering to the subject the RAS(ON) multi-selective inhibitor described herein. Accordingly, one embodiment of the present disclosure provides a method treating a subject in need thereof by administering a pharmaceutical composition containing the RAS(ON) multi-selective inhibitor described herein, and a pharmaceutically acceptable excipient, as well as methods of using the RAS(ON) inhibitor therapy to prepare such compositions.

As used herein, the term “pharmaceutical composition” refers to a compound, such as a RAS(ON) multi-selective inhibitor of the disclosure, formulated together with a pharmaceutically acceptable excipient.

In some embodiments, a RAS(ON) multi-selective inhibitor of the disclosure is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or nonaqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

For use as treatment of subjects, a RAS(ON) multi-selective inhibitor of the disclosure can be formulated as pharmaceutical compositions. Depending on the subject to be treated, the mode of administration, and the type of treatment desired, e.g., prevention, prophylaxis, or therapy, a RAS(ON) multi-selective inhibitor of the disclosure is formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from 0.1% to 99%, from 5% to 90%, or from 1% to 20% of a RAS(ON) multi-selective inhibitor of the disclosure, by weight or volume. In some embodiments, a RAS(ON) multi-selective inhibitor of the disclosure may be present in amounts totaling 1-95% by weight of the total weight of a composition, such as a pharmaceutical composition.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art. In one embodiment the therapeutically effective amount of a RAS(ON) multi-selective inhibitor of the disclosure is administered orally in the form of a tablet or multiple tablets.

A RAS(ON) multi-selective inhibitor of the disclosure, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein a RAS(ON) multi-selective inhibitor of the disclosure is mixed with an inert solid diluent (e.g., potato starch, lactose, microcrystalline cellulose, calcium carbonate, calcium phosphate or kaolin), or as soft gelatin capsules wherein a RAS(ON) multi-selective inhibitor of the disclosure is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating a RAS(ON) multi-selective inhibitor of the disclosure into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which a RAS(ON) multi-selective inhibitor of the disclosure, or a composition thereof, can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that a RAS(ON) multi-selective inhibitor of the disclosure and pharmaceutical compositions thereof can be formulated and employed in combination therapies, that is, a RAS(ON) multi-selective inhibitor of the disclosure and pharmaceutical compositions thereof can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In some embodiments, the invention discloses a method of treating a disease or disorder that is characterized by aberrant RAS activity due to a RAS mutant. In some embodiments, the disease or disorder is a cancer.

Accordingly, also provided is a method of treating cancer in a subject in need thereof, the method comprising administering to the subject a therapeutically effective amount of a RAS(ON) multi-selective inhibitor of the disclosure or a pharmaceutical composition comprising such a compound. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a RAS protein-related disorder in a subject in need thereof, the method comprising administering to the subject a therapeutically effective amount of a RAS(ON) multi-selective inhibitor of the present invention, or a pharmaceutically acceptable salt thereof, or a pharmaceutical composition comprising such a compound or salt.

In some embodiments, a RAS(ON) multi-selective inhibitor of the disclosure, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the RAS protein is wild-type. (RAS^{WT}). Accordingly, in some embodiments, a RAS(ON) multi-selective inhibitor of the present invention is employed in a method of treating a patient having a cancer comprising a RAS^{WT} (e.g., KRAS^{WT}, HRAS^{WT} or NRAS^{WT}). In some embodiments, the RAS protein is RAS amplification (e.g., KRAS^{amp}). Accordingly, in some embodiments, a RAS(ON) multi-selective inhibitor of the present invention is employed in a method of treating a patient having a cancer comprising a RAS^{amp} (KRAS^{amp}, HRAS^{amp} or NRAS^{amp}). In some embodiments, the cancer comprises a RAS mutation, such as a RAS mutation described herein. In some embodiments, the cancer comprises a KRAS G12C mutation. In some embodiments, a mutation is a G12C mutation, and one or more mutations selected from:
(a) the following KRAS mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following HRAS mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following NRAS mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
or a combination of any of the foregoing. In some embodiments, the cancer comprises a RAS mutation selected from the group consisting of G12C, G13C, G12A, G12D, G13D, G12S, G13S, G12V and G13V. In some embodiments, the cancer comprises at least two RAS mutations selected from the group consisting of G12C, G13C, G12A, G12D, G13D, G12S, G13S, G12V and G13V. In some embodiments, a RAS(ON) multi-selective inhibitor of the present invention inhibits more than one RAS mutant. In some embodiments, the mutation is selected from the group consisting of G12A, G12C, G12D, G12E, G12F, G12H, G12I, G12K, G12L, G12M, G12N, G12P, G12Q, G12R, G12S, G12T, G12V, G12W and G12Y, or a combination thereof, of KRAS, NRAS or HRAS. In some embodiments, the mutation is selected from the group consisting of G12H, G12I, G12K, G12M, G12N, G12P, G12Q, G12T, G12W, and G12Y, or a combination thereof, of KRAS, NRAS or HRAS. In some embodiments, the cancer is non-small cell lung cancer and the RAS mutation comprises a KRAS mutation, such as KRAS G12C. In some embodiments, the cancer is colorectal cancer and the RAS mutation comprises a KRAS mutation, such as KRAS G12C. In some embodiments, the cancer is pancreatic cancer and the RAS mutation comprises an KRAS G12C mutation. In some embodiments, the cancer is non-small cell lung.

Additionally, in some embodiments, the cancer comprises a KRAS mutation selected from the group consisting of G12C, G12D, G13C, G12V, G13D, G12R, G12S, Q61H, Q61K and Q61L. In some embodiments, the cancer comprises an NRAS mutation selected from the group consisting of G12C, Q61H, Q61K, Q61L, Q61P and Q61R. In some embodiments, the cancer comprises a RAS mutation selected from the group consisting of G12C, G13C, G12A, G12D, G13D, G12S, G13S, G12V and G13V.

In some embodiments, the cancer comprises at least two RAS mutations selected from the group consisting of G12C, G13C, G12A, G12D, G13D, G12S, G13S, G12V and G13V. In some embodiments, a RAS(ON) multi-selective inhibitor of the present invention inhibits more than one RAS mutant. For example, a compound may inhibit both KRAS G12C and KRAS G13C. A compound may inhibit both NRAS G12C and KRAS G12C.

Methods of detecting RAS mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer and the RAS mutation comprises a KRAS mutation, such as KRAS G12C, KRAS G12V or KRAS G12D. In some embodiments, the cancer is colorectal cancer and the RAS mutation comprises a KRAS mutation, such as KRAS G12C, KRAS G12V or KRAS G12D. In some embodiments, the cancer is pancreatic cancer and the RAS mutation comprises an KRAS mutation, such as KRAS G12C. In some embodiments, the cancer is melanoma. In some embodiments, the cancer is non-small cell lung cancer.

In some embodiments, a cancer comprises a RAS mutation and an STK11^{LOF} a KEAP1, an EPHA5 or an NF1 mutation, or a combination thereof. In some embodiments, the cancer is non-small cell lung cancer and comprises a KRAS G12C mutation. In some embodiments, the cancer is non-small cell lung cancer and comprises a KRAS G12C mutation, an STK11^{LOF} mutation, and a KEAP1 mutation. In some embodiments, the cancer is non-small cell lung cancer and comprises a KRAS G12C mutation and an STK11^{LOF} mutation. In some embodiments, the cancer is non-small cell lung cancer and comprises a KRAS G12C mutation and an STK11^{LOF} mutation. In some embodiments, a cancer comprises a KRAS G13C RAS mutation and an STK11^{LOF} a KEAP1, an EPHA5 or an NF1 mutation. In some embodiments, the cancer is colorectal cancer and comprises a KRAS G12C mutation. In some embodiments, the cancer is pancreatic cancer and comprises a KRAS G12C mutation. In some embodiments, the cancer is endometrial cancer and comprises a KRAS G12C mutation. In some embodiments, the cancer is gastric cancer and comprises a KRAS G12C mutation.

In one aspect, the present disclosure provides methods for treating a RAS protein-related disorder in a subject where the RAS-related disorder pathology is mediated, in part, through increased signaling in the RAS/MAPK pathway. In various embodiments, the method generally comprises administering to the subject a therapeutically effective amount of a RAS(ON) multi-selective inhibitor of the disclosure. In some embodiments, the RAS protein-related disorder is a RASopathy. A RASopathy is a group of genetic disorders that are caused by mutations in genes involved in the RAS/MAPK signaling pathway. RASopathies are characterized by a range of clinical features and can affect multiple organ systems, including the cardiovascular, musculoskeletal, neurological, and dermatological systems.

In one aspect, the present disclosure is directed to methods of treating a disease or disorder that is characterized by aberrant RAS activity (e.g., cancer or a RASopathy). In some embodiments the disease or disorder is cancer (e.g., a cancer having one or more RAS mutations that cause aberrant RAS activity). Non-limiting examples of non-cancerous RAS related diseases or disorders are shown in Table 1. In each embodiment, the method generally comprises administering to the subject a therapeutically effective amount of a RAS(ON) multi-selective inhibitor of the disclosure. In some embodiments, the methods comprise administering RAS(ON) multi-selective inhibitor of the disclosure in combination with one or more therapeutic agents. Suitable RAS(ON) multi-selective inhibitor of the disclosure and additional therapeutic agents are described herein.

Exemplary RAS related non-cancerous indications are summarized in Table 1.

TABLE 1
Exemplary RAS related Non-cancerous Indications

| Disease or disorder | | References |
|---|---|---|
| Immune | Autoimmune | Journal of Clinical Immunology vol. 35, |
| disease | disease | pp. 454-458 (2015) |
| | Rheumatoid | The Open Rheumatoid Journal vol. 6, pp. |
| | arthritis | 259-272 (2012) |
| | RAS-related | PNAS vol. 104, pp. 8953-8958 (2007) |
| | autoimmune | Blood vol. 117, pp. 2887-2890 (2011) |
| | lympho- | |
| | proliferative | |
| | disorders | |
| Infection | Influenza | Cancer Research vol. 61, pp. 8188-8193 |
| | | (2001) |
| | | PloS ONE vol. 6, el6324 (2011) |
| | | Seikagaku: The Journal of the Japanese |
| | | Biochemical Society vol. 87, Issue 1 |
| | EBV infection | Oncogene vol. 23, pp. 8619-8628 (2004) |
| | HIV infection | Journal of Biological Chemistry vol. 275, |
| | | pp. 16513-16517 (2000) |
| Neurologic | Alzheimer's | Biochimica et Biophysica Acta vol. 1802, |
| disease | disease | pp. 396-405 (2010) |
| | | Neurobiology of Disease vol. 43, pp. 38- |
| | | 45 (2011) |
| | Parkinson's | Biochimica et Biophysica Acta vol. 1802, |
| | disease | pp. 396-405 (2010) |
| | ALS | Biochimica et Biophysica Acta vol. 1802, |
| | | pp. 396-405 (2010) |
| RAS/MAPK | Noonan | Human Molecular Genetics vol. 15, pp. |
| syndrome | Syndrome | R220-R226 (2006) |
| | Costello | Genetics in Medicine vol. 14, pp. 285- |
| | syndrome | 292 (2012) |
| | CFC syndrome | Human Mutation vol. 29, pp. 992-1006 |
| | | (2008) |
| Other | Cirrhosis/ | Gastroenterologia Japonica vol. 24, pp. |
| diseases or | Chronic | 270-276 (1989) |
| disorders | hepatitis | |
| | Memory | Nature Communications vol. 7, 12926 |
| | impairment | (2016) |
| | Fibrosis | WO 2025080653 |

In some embodiments, the methods include treating a RASopathy selected from Noonan syndrome, Costello syndrome, cardiofaciocutaneous syndrome, neurofibromatosis type 1, and Legius syndrome. While each RASopathy has unique features, they all share certain similarities, such as facial dysmorphisms, cardiac abnormalities, developmental delays, and an increased risk of certain cancers.

RASopathies are typically diagnosed through a combination of clinical evaluation, genetic testing, and imaging studies. Treatment and management of RASopathies depend on the specific type and severity of the disorder, but may include medication, surgery, and supportive therapies such as physical and occupational therapy.

### Combination Therapies

The methods of the disclosure may include a RAS(ON) multi-selective inhibitor of the disclosure in combination an additional therapeutic agent (e.g., a second RAS inhibitor such as a pan-KRAS inhibitor). The combination therapy can be administered on an intermittent dosing regimen such that the RAS(ON) multi-selective inhibitor is administered intermittently as described above, and the additional therapeutic agent is administered every day of the dosing regimen. Alternatively, the RAS(ON) multi-selective inhibitor and the second therapeutic agent can be administered on alternating days such that one agent is administered on a day the other agent is not administered.

The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

In certain embodiments, compositions of the disclosure comprise a RAS(ON) multi-selective inhibitor of the present disclosure and one additional therapeutic agent. In certain embodiments, compositions of the disclosure comprise a RAS(ON) multi-selective inhibitor of the present invention and two additional therapeutic agents. In certain embodiments, compositions of the disclosure comprise a RAS(ON) multi-selective inhibitor of the present invention and three additional therapeutic agents. In certain embodiments, compositions of the disclosure comprise a RAS(ON) multi-selective inhibitor of the present invention and four or more additional therapeutic agents.

Also provided are pharmaceutical compositions including the combinations, or a pharmaceutically acceptable salt thereof, and a pharmaceutically acceptable excipient. Compositions comprising a combination of therapeutic agents may be used in methods of modulating RAS (e.g., in a subject or in a cell) and in methods of treating RAS related diseases and disorders (e.g., cancer), as described herein. The present disclosure provides, inter alia, compositions, methods, and kits for treating or preventing a RAS related disease or disorder.

Exemplary agents that may be used in combination with a RAS(ON) multi-selective inhibitor of the present disclosure are described below. All references herein are incorporated by reference for the agents described, including compound or molecular structures disclosed therein, whether explicitly stated as such or not.

In certain embodiments, the present disclosure provides methods of administering the combination of a RAS(ON) multi-selective inhibitor of the disclosure and a pan-KRAS inhibitor, where the RAS(ON) multi-selective inhibitor is administered on an intermittent dosing regimen as described herein and the pan-KRAS inhibitor is administered on a continuous dosing regimen (e.g., daily administration such as QD or BID) or on an intermittent dosing regimen.

In some embodiments, a pan-KRAS inhibitor is selected from one disclosed in any of the following: WO 2025106905, WO 2025106901, WO 2025101776, WO 2025096738, WO 2025092798, WO 2025085748, WO 2025077770, WO 2025077663, WO 2025076523, WO 2025064848, WO 2025059366, WO 2025059040, WO 2025049641, WO 2025049619, WO 2025049402, WO 2025045141, WO 2025038936, WO 2025026903, WO 2025016899, WO 2025007000, WO 2025006967, WO 2025006962, WO 2025006720, WO 2025006704, WO 2024255795, WO 2024254404, WO 2024246099, WO 2024238633, WO 2024238343, WO 2024236452, WO 2024235286, WO 2024235225, WO 2024230734, WO 2024220645, WO 2024220532, WO 2024218686, WO 2024215754, WO 2024213979, WO 2024213122, WO 2024209339, WO 2024206766, WO 2024206747, WO 2024192424, WO 2024178313, WO 2024178304, WO 2024173842, WO 2024153180, WO 2024119277, WO 2024120433, WO 2024115890, WO 2024112654, WO 2024104453, WO 2024104425, WO 2024107686, WO 2024104453, WO 2024103010, WO 2024085661, WO 2024083246, WO 2024083168, WO 2024067575, WO 2024064335, WO 2024063578, WO 2024063576, WO 2024051852, WO 2024051763, WO 2024046370, WO 2024044667, WO 2024041621, WO 2024041606, WO 2024041589, WO 2024040131, WO 2024040109, WO 2024032747, WO 2024032704, WO 2024032703, WO 2024032702, WO 2024031088, WO 2024030647, WO 2024030633, WO 2024015262, WO 2024009191, WO 2024008068, WO 2024002373, WO 2023287896, WO 2023274324, WO 2023246914 (e.g., compound 14), WO 2023246777, WO 2023230190, WO 2023215802, WO 2023215801, WO 2023197984, WO 2023190748, WO 2023183585, WO 2023179703, WO 2023173017, WO 2023173016, WO 2023173014, WO 2023172737, WO 2023154766, WO 2023143352, WO 2023143312, WO 2023138589, WO 2023133183, WO 2023122662, WO 2023114733, WO 2023099624, WO 2023099623, WO 2023099612, WO 2023099608, WO 2023099592, WO 2023097227, WO 2023064857, WO 2023056421, WO 2023049697, WO 2023046135, WO 2023039240, WO 2023034290, WO 2023020523, WO 2023020521, WO 2023020519, WO 2023020518, WO 2023001123, WO 2022271823, WO 2022261210, WO 2022258974, WO 2022256459, WO 2022250170, WO 2022248885, WO 2022228543, WO 2022216762, WO 2022072783, WO 2016161361, KR 20240101190, KR 20240101189, KR 20240041720, KR 20240041719, CN 119751476, CN 119661539, CN 119371353, CN 119019382, CN 118791505, CN 118221700, CN 118126064, CN 117924327, CN 117946135, CN 117800990, CN 117800989, CN 117683051, CN 117486901, CN 117263959, CN 116969977, or CN 116332948, each of which is incorporated herein by reference in its entirety, including the compound structures disclosed therein. In some embodiments, combination comprising a pan-KRAS inhibitor therapy comprises ERAS-4001. In some embodiments, the pan-KRAS inhibitor is a pan-KRAS inhibitor in a patent application filed in the name of Medshine Discovery, Inc. In some embodiments, a combination comprising a pan-KRAS inhibitor therapy includes A2A-03, ABREV01, ADT-007, ABT-200, ADT-030, ADT-1004, BBP-454, BGB-53038, BI-2865, BI-2493, BI 3706674, BRSD-143, ERAS-4, ERAS-254, ERAS-4001, HB-700 (G12X+G13D), HZ-V068, ID12241161, JAB-23400, LY4066434, OC211, PF-07985045, PF-07934040, QTX2024, QTX3034, RSC-1255, SIL204, SYNB021225, YL-17231, ZG2001. In some embodiments, the pan-KRAS is selected from one disclosed in WO 2023246914. In one embodiment, the methods of the present disclosure include administering the combination of the RAS(ON) multi-selective inhibitor, ERAS-0015, and the pan-KRAS inhibitor, ERAS-4001 using the methods described herein.

Compositions and methods described herein may include one or more RAS(ON) mutant-selective inhibitors. Numerous RAS(ON) mutant-selective inhibitors have been disclosed.

In some embodiments, the RAS(ON) mutant-selective inhibitor is a RAS(ON) G12C-selective inhibitor. In some embodiments, the RAS(ON) mutant-selective inhibitor is a RAS(ON) G12D-selective inhibitor. In some embodiments, the RAS(ON) mutant-selective inhibitor is a RAS(ON) G13C-selective inhibitor. In some embodiments, the RAS(ON) mutant-selective inhibitor is a RAS(ON) Q61H-selective inhibitor. In some embodiments, the RAS(ON) mutant-selective inhibitor is a RAS(ON) G12V-selective inhibitor. In some embodiments, the RAS(ON) mutant-selective inhibitor is a RAS(ON) G13D-selective inhibitor. In some embodiments, the RAS(ON) mutant-selective inhibitor is a RAS(ON) G12R-selective inhibitor.

RAS(ON) mutant-selective inhibitors useful in combinations according to the methods of the present disclosure can be found in any one of the following patent applications: WO 2025104149, WO 2025093625, WO 2025080946, WO 2024249299, WO 2024211663, WO 2024211712, WO 2024208934, WO 2024149819, WO 2024008610, WO 2024102421, WO 2023240263, WO 2023133543, WO 2023015559, WO 2023086341, WO 2023208005, WO 2023232776, WO 2023086341, WO 2023060253, WO 2023015559, WO 2022235870, WO 2022235864, WO 2021091967, WO 2021091982, WO 2021108683, WO 2020132597, each of which is incorporated herein by reference in its entirety, including the compound structures disclosed therein.

In some embodiments, the RAS(ON) mutant-selective inhibitor useful according to the present disclosure is a G12D-selective inhibitor, such as zoldonrasib (RMC-9805) or RMC-9945.

In some embodiments, the RAS(ON) mutant-selective inhibitor is a G12C-selective inhibitor, such as elironrasib (RMC-6291) or RMC-4998.

In some embodiments, the RAS(ON) mutant-selective inhibitor is a G12V-selective inhibitor, such as RMC-5127.

In some embodiments, the RAS(ON) mutant-selective inhibitor is a G13C-selective inhibitor, such as RMC-8839. In some embodiments, the RAS(ON) mutant-selective inhibitor is a Q61H-selective inhibitor, such as RMC-0708. In some embodiments, the RAS(ON) mutant-selective inhibitor is a G12R-selective inhibitor, such as RMC-8264.

The RAS(ON) inhibitor compounds described herein may be made from commercially available starting materials or synthesized using known organic, inorganic, or enzymatic processes. By way of example, the RAS(ON) compounds can be synthesized using the methods described in WO 2022060836, WO 2021091956, or WO 2021091982, or any of the other RAS(ON) references cited herein, together with synthetic methods known in the art of synthetic organic chemistry, or variations thereon as appreciated by those skilled in the art.

A RAS(ON) inhibitor may be an antibody-drug conjugate, such as WO 2025051241 and WO 2024189481. See also doi.org/10.1021/acs.jmedchem.4c02929.

In some embodiments, the combination therapy comprising a RAS(ON) multi-selective inhibitor of the present disclosure may include one or more RAS(ON) inhibitors, for example, a RAS(ON) multi-selective inhibitor of the present disclosure plus one or more RAS(ON) multi-selective inhibitors and/or one or more RAS(ON) mutant-selective inhibitors.

### a) RAS/MAPK Inhibitors

Compositions and methods described herein may include a RAS(ON) multi-selective inhibitor of the present disclosure in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS(ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); R05126766 (Roche, described in PLoS One. 2014 Nov. 25; 9(11)); and GSK1120212 (or JTP-74057, described in Clin Cancer Res. 2011 Mar. 1; 17(5):989-1000). The MAPK pathway inhibitor may be PLX8394, LXH254, GDC-5573, or LY3009120. A MAPK pathway inhibitor may be a PI3Kα:RAS breaker, such as BBO-10203.

### i) RAS(OFF) Inhibitors, RAS(OFF) Degraders and Other RAS Inhibitor Types

Compositions and methods described herein may include a RAS(ON) multi-selective inhibitor of the present disclosure in combination with one or more RAS(OFF) inhibitors. Numerous mutant-selective and pan-KRAS inhibitors have been disclosed and are known in the art. A RAS(OFF) inhibitor may be administered or formulated in combination with a RAS(ON) inhibitor described herein. RAS(OFF) inhibitors are designed to inhibit RAS activity by targeting different regions of the RAS protein in its inactive state (GDP bound state), preventing its activation and downstream signaling.

In some embodiments, a RAS(OFF) inhibitor is a KRAS(OFF) inhibitor that has a molecular weight of under 700 Da. In some embodiments, the KRAS(OFF) inhibitor is specific for a KRAS^{G12C} mutation. KRAS^{G12C}(OFF) inhibitors use a covalent binding group that allows them to selectively target the KRAS^{G12C} mutant protein, and many such inhibitors comprise a pyrimidine core. KRAS^{G12C}(OFF) inhibitors all target the same cysteine residue in the KRAS^{G12C} mutant protein, leading to a conformational change that locks the protein in an inactive state. KRAS^{G12C}(OFF) inhibitors include, but are not limited to, adagrasib (MRTX849), divarasib (RG6330/GDC-6036), fulzerasib (IB1351/GFH925), garsorasib (D-1553), glecirasib (JAB-21822), olomorasib (LY3537982), opnurasib (JDQ443), sotorasib (AMG 510), ARS-853, ARS-1620, BI-0474, BI 1823911, BPI-421286, D3S-001, ERAS-3490, GEC255, GH35, HBI-2438, HS-10370, JAB-21000, JAB-21822, JMKX001899, JNJ-74699157 (ARS-3248), MK-1084, SK-17, and YL-15293. In some embodiments, the KRAS(OFF) inhibitor is selected from AMG510 and MRTX849. In some embodiments, the KRAS(OFF) inhibitor is AMG510. In some embodiments, the KRAS(OFF) inhibitor is MRTX849. In some embodiments, the KRAS(OFF) inhibitor is GDC-6036. A RAS(OFF) inhibitor may be an antibody-drug conjugate. See also doi.org/10.1021/acs.jmedchem.4c02929.

In some embodiments, a KRAS(OFF) inhibitor is specific for a KRAS^{G12D} mutation. Non-limiting examples of KRAS^{G12D}(OFF) inhibitors include ASP3082, AST2169, BPI-501836, DN022150, ERAS-4693, ERAS-5024, GDC-7035 (RG6620), HBW-012-D, HBW-012-E, HBW-012336, HRS-4642, HS-10529, INCB186748, JAB-22000, KD-8, KRB-456, LY3962673, MRTX282, MRTX1133, Q2a, QLC1101, RNK08954, SHR1127, TH-Z827, TH-Z835, TSN1611, and VRTX153.

In some embodiments, the small molecule RAS(OFF) inhibitor is specific for a KRAS^{G12V} mutation (e.g., JAB-23000, QTX3544). In some embodiments, the small molecule RAS(OFF) inhibitor is specific for a KRAS^{G13D} mutation.

In some embodiments, reference to the term RAS(OFF) inhibitor includes any such RAS(OFF) inhibitor disclosed in any one of the following patent applications: WO 2025111586, WO 2025111582, WO 2025108443, WO 2025106905, WO 2025106901, WO 2025101776, WO 2025096984, WO 2025096957, WO 2025096738, WO 2025092986, WO 2025092798, WO 2025085748, WO 2025085580, WO 2025080653, WO 2025077770, WO 2025077663, WO 2025076523, WO 2025072649, WO 2025072457, WO 2025072451, WO 2025067459, WO 2025067453, WO 2025064848, WO 2025064542, WO 2025061125, WO 2025059366, WO 2025059040, WO 2025054530, WO 2025054347, WO 2025054270, WO 2025053850, WO 2025051242, WO 2025045141, WO 2025049641, WO 2025049619, WO 2025049402, WO 2025049274, WO 2025040767, WO 2025038936, WO 2025036475, WO 2025036470, WO 2025034883, WO 2025034849, WO 2025026903, WO 2025019688, WO 2025018418, WO 2025016899, WO 2025016432, WO 2025011443, WO 2025010415, WO 2025007000, WO 2025006967, WO 2025006962, WO 2025006720, WO 2025006704, WO 2025002430, WO 2025002302, WO 2024259169, WO 2024254404, WO 2024254334, WO 2024255795, WO 2024246099, WO 2024243025, WO 2024238633, WO 2024238343, WO 2024236452, WO 2024235286, WO 2024235225, WO 2024233776, WO 2024230734, WO 2024230707, WO 2024229447, WO 2024229444, WO 2024229442, WO 2024229317, WO 2024227091, WO 2024220645, WO 2024220532, WO 2024218686, WO 2024215862, WO 2024215754, WO 2024213979, WO 2024213122, WO 2024208305, WO 2024209339, WO 2024206766, WO 2024206747, WO 2024197503, WO 2024193698, WO 2024192424, WO 2024179546, WO 2024178313, WO 2024178304, WO 2024173842, WO 2024167922, WO 2024160225, WO 2024159471, WO 2024159470, WO 2024158778, WO 2024158242, WO 2024153119, WO 2024153116, WO 2024138486, WO 2024138206, WO 2024138052, WO 2024131829, WO 2024125642, WO 2024125600, WO 2024123913, WO 2024123102, WO 2024120433, WO 2024120419, WO 2024119277, WO 2024118926, WO 2024109233, WO 2024112654, WO 2024104453, WO 2024104425, WO 2024107686, WO 2024104453, WO 2024103010, WO 2024097559, WO 2024091409, WO 2024088273, WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO 2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, KR 20240159370, KR 20240101190, KR 20240101189, KR 20240041720, KR 20240041719, CN 119930639, CN 119909188, CN 119751476, CN 119733053, CN 119684316, CN 119684315, CN 119684314, CN 119661556, CN 119661555, CN 119661539, CN 119606974, CN 119528902, CN 119528810, CN 119504612, CN 119490514, CN 119490512, CN 119462648, CN 119371353, CN 119350242, CN 119264124, CN 119241566, CN 119060049, CN 119060066, CN 119019382, CN 118994158, CN 118994031, CN 118806919, CN 118791505, CN 118772176, CN 118754899, CN 118745175, CN 118666870, CN 118666869, CN 118580238, CN 118307563, CN 118221700, CN 118221699, CN 118221698, CN 118221685, CN 118126064, CN 118078802, CN 118078801, CN 118005656, CN 117986263, CN 117986263, CN 117946135, CN 117924327, CN 117903117, CN 117800990, CN 117800989, CN 117800976, CN 117736226, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115724842, CN 115724842, CN 115721720, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113321654, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, CN 109574871, US 2025115603, US 2025114346, US 2025114339, US 20240358702, US 2024270736, or EP 4389751, each of which is incorporated herein by reference in its entirety, including the RAS compound structures disclosed therein which are specifically incorporated herein by reference.

In some embodiments, a RAS inhibitor binds to the OFF form as well as the ON form. Non-limiting examples of such inhibitors include, e.g., pan-KRAS: ALTA3263, AMG 410, BBO-11818, HBW-016-K, HEC211909, JAB-23E73, JAB-23425, JAB-23E73; G12C: BBO-8520, FMC-376; G12D: AZD0022, GFH375 (VS-7375), INCB161734, QTX3046, TSN1611 and TH-Z835.

In some embodiments, a RAS inhibitor binds to the ON form of RAS but is not a tri-complex inhibitor, such as pan-KRAS inhibitors JTX-102 and JTX-105.

In any embodiment employing a RAS(OFF) inhibitor herein, a RAS(OFF) degrader targeting the OFF state of RAS may be employed. These degraders are known in the art. RAS degraders may be found, for example, in one or more of the following applications: WO 2025108479, WO 2025107579, WO 2025103476, WO 2025096855, WO 2025085815, WO 2025083472, WO 2025078984, WO 2025076044, WO 2025058008, WO 2025053850, WO 2025024732, WO 2025019823, WO 2025006783, WO 2025006753, WO 2024263586, WO 2024261257, WO 2024261256, WO 2024241248, WO 2024233838, WO 2024199266, WO 2024188281, WO 2024/59164, WO 2024152247, WO 2024149214, WO 2024131777, WO 2024120424, WO 2024119278, WO 2024118966, WO 2024118960, WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023215906, WO 2023185864, WO 2023171781, WO 2023141570, WO 2023138524, WO 2023130012, WO 2023116934, WO 2023099620, WO 2023081476, WO 2023077441, WO 2022260482, CN 119219669, CN 119161349, CN 118955610, CN 118772249, CN 118725012, CN 118496502, CN 118496300, CN 118126040, or CN 115785199, each of which is incorporated herein by reference in its entirety. Non-limiting examples of RAS degraders include: ASP3082 (KRAS G12D); ASP4396 (KRAS G12D); BPI-585725 (G12X and WT), LT-010366 (G12D); PT0253 (G12D), RD0255359 (KRAS G12C/D/V); RP03707 (G12D).

In some embodiments, the RAS(OFF) inhibitor is a peptide-based inhibitor. Peptide-based RAS(OFF) inhibitors have been developed that target specific regions of the RAS protein, such as the Switch II region or the RAS-effector interface. Non-limiting examples include the K-Ras-binding peptide (Krpep-2d), the Ras inhibitory peptide (Rasin) and LUNA18 (NCT05012618). Peptide-based RAS(OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways. See, e.g., WO 2025018418, WO 2024219480, WO 2024219446, WO 2024176153, WO 2024101402, WO 2024101386, WO 2023214576, WO 2023140329, WO 2022234853, WO 2022234852, WO 2022234851, WO 2022234639 and CN 120040551, each of which is incorporated herein by reference in its entirety.

Peptide-based RAS(OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS(OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS(OFF) inhibitors include the Ras-binding domain (RBD) of other RAS-interacting proteins such as RalGDS and SOS, as well as sequences that mimic the structure of the switch regions of RAS itself. These motifs are typically used to optimize the binding affinity and selectivity of the inhibitor for the desired target protein or interaction.

In some embodiments, the RAS(OFF) inhibitor is an antibody or antigenic binding peptide specific for RAS(OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS(OFF)-specific inhibitory antibodies include anti-p21ser, and K27 (DARPin) (see, e.g., Khan et al, Biochim Biophys Acta Mol Cell Res. 2020 February; 1867(2):118570). See also WO 2024136608 and WO 2024111590, each of which is incorporated herein by reference in its entirety.

Antibody-drug conjugates may also be constructed using RAS inhibitors (e.g., RAS(OFF) inhibitors), such as WO 2024189481, which is incorporated herein by reference in its entirety, including the compound structures disclosed therein.

Vaccines may also be used in combination with compounds of the present invention. Non-limiting examples include: AFNT-111 (KRAS G12V), AFNT-211 (KRAS G12V), AFNT-212 (KRAS G12D), ELI-002 (KRAS G12/13X), HB-700, NT-112 (KRAS G12D), and TG01 (pan-KRAS).

Other RAS modalities useful in combination with compounds of the present invention include: ADGN-123, ADGN-121 (gene editing peptide-RNA nanoparticles G12D); ADT-030 (Ras/B-catenin inhibitor); BBO-10203 (PI3Kα:RAS breaker); BI 1701963 (pan-KRAS:SOS1); mRNA-5671 (nucleic acid) and RO7673396 (RAS inhibitor).

### ii) SOS1 Inhibitors

In some embodiments, compositions and methods described herein may include a RAS(ON) multi-selective inhibitor of the present disclosure in combination with one or more SOS1 inhibitors. A SOS1 inhibitor may be administered or formulated in combination with a RAS(ON) multi-selective inhibitor of the present disclosure and/or any additional therapeutic agent described herein. In some embodiments, a SOS1 inhibitor is one or more of RMC-5845, RMC-4948, RMC-0331, BI-1701963, BI-1918455, BI-3406, SDR5, MRTX-0902, ZG2001, and BAY-293. In some embodiments, reference to the term SOS1 inhibitor includes any such SOS1 inhibitor disclosed in any one of the following patent applications: WO 2025070947, WO 2025067316, WO 2025062157, WO 2025059046, WO 2025038785, WO 2025003694, WO 2025000265, WO 2024255827, WO 2024172632, WO 2024172631, WO 2024119028, WO 2024102952, WO 2024083257, WO 2024083255, WO 2024079252, WO 2024075070, WO 2024067744, WO 2024035921, WO 2024027762, WO 2024008185, WO 2023250165, WO 2023215257, WO 2023215256, WO 2023180345, WO 2023109929, WO 2023059597, WO 2023041049, WO 2023029833, WO 2023022497, WO 2022184116, WO 2022171184, WO 2022170952, WO 2022170917, WO 2022170802, WO 2022161461, WO 2022157629, WO 2022139304, WO 2022121813, WO 2022028506, WO 2021228028, WO 2019122129, KR 20240128541, CN 119431234, CN 119039237, CN 119039234, CN 118812510, CN 117800922, CN117143175, CN 117143176, CN 116462669, CN 116444447, CN 115806560, CN 115677702, CN 115215847, CN 115028644, CN 114685488, and CN 111393519 each of which is incorporated herein by reference in its entirety, including the compound structures disclosed therein which are specifically incorporated herein by reference.

iii) SHP Inhibitors

In some embodiments, compositions and methods described herein may include a RAS(ON) multi-selective inhibitor of the present disclosure in combination with one or more SHP inhibitors. A SHP inhibitor may be administered or formulated in combination with a RAS(ON) multi-selective inhibitor of the present disclosure and/or any additional therapeutic agent described herein. In some embodiments, the SHP inhibitor is an inhibitor of SHP1. In some embodiments, the SHP inhibitor is an inhibitor of SHP2. In some embodiments, the SHP1 inhibitor is SB8091 or SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, ARRY-558, or BBP-398. In some embodiments, reference to the term SHP2 inhibitor includes any such SHP2 inhibitor disclosed in any one of the following patent applications: WO 2025075693, WO 2025019666, WO 2025011568, WO 2025011480, WO 2024258652, WO 2024193439, WO 2024175081, WO 2024147703, WO 2024125603, WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 116332908, CN 119264153, CN 117069698, CN 117143107, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, each of which is incorporated herein by reference in its entirety, including the compound structures disclosed therein which are specifically incorporated herein by reference.

### iv) MEK Inhibitors

In some embodiments, compositions and methods described herein may include a RAS(ON) multi-selective inhibitor of the present disclosure in combination with one or more MEK inhibitors. A MEK inhibitor may be administered or formulated in combination with a RAS(ON) multi-selective inhibitor of the present disclosure and/or any additional therapeutic agent described herein. In some embodiments, a MEK inhibitor is one or more of pimasertib, IMM-1-104, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; AF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US 2023032403, and CN 115813930, each of which is incorporated herein by reference in its entirety, including the compound structures disclosed therein which are specifically incorporated herein by reference.

### v) RAF Inhibitors

In some embodiments, compositions and methods described herein may include a RAS(ON) multi-selective inhibitor of the present disclosure in combination with one or more RAF inhibitors. A RAF inhibitor may be administered or formulated in combination with a RAS(ON) multi-selective inhibitor of the present disclosure and/or any additional therapeutic agent described herein. In some embodiments, a RAF inhibitor is VS-6766 or BTDX-4933. In some embodiments, a RAF inhibitor is a BRAF inhibitor. BRAF inhibitors that may be used in combination with a RAS(ON) multi-selective inhibitor of the present disclosure include, for example, VS-6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E. In some embodiments, reference to the term RAF inhibitor includes any such RAF inhibitor disclosed in any one of the following patent applications: WO 2023076991, WO 2022226626, WO 2022226261, WO 2019084459, WO 2018203219, WO 201851306, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.

### vi) ERK Inhibitors

In some embodiments, compositions and methods described herein may include a RAS(ON) multi-selective inhibitor of the present disclosure in combination with one or more ERK inhibitors. An ERK inhibitor may be administered or formulated in combination with a RAS(ON) multi-selective inhibitor of the present disclosure and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or 1-75. In some embodiments, reference to the term ERK inhibitor includes any such ERK inhibitor disclosed in any one of the following patent applications: WO 2023076305, WO 2022259222, WO 2022221547, WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN114315837, CN 115057860, CN 107973783, each of which is incorporated herein by reference in its entirety, including the compound structures disclosed therein which are specifically incorporated herein by reference.

vii) MAPK Inhibitors

In some embodiments, compositions and methods described herein may include a RAS(ON) multi-selective inhibitor of the present disclosure in combination with one or more Mitogen-Activated Protein Kinase (MAPK) inhibitors. A MAPK inhibitor may be administered or formulated in combination with a RAS(ON) multi-selective inhibitor of the present disclosure and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapidmod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with a RAS(ON) multi-selective inhibitor of the present disclosure is an inhibitor of MAP2K4. A non-limiting example of a MAP2K4 inhibitor useful according to the disclosure is HRX-0233.

### b) Kinase Inhibitors

Compositions and methods described herein may include a RAS(ON) multi-selective inhibitor of the present disclosure in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

### i) PKA Inhibitors

In some embodiments, compositions and methods described herein may include one or more Protein Kinase A (PKA) inhibitors. A PKA inhibitor may be administered or formulated in combination with a RAS(ON) multi-selective inhibitor of the present disclosure and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

### ii) FAK Inhibitors

In some embodiments, compositions and methods described herein may include a RAS(ON) multi-selective inhibitor of the present disclosure in combination with one or more Focal Adhesion Kinase (FAK) inhibitors. A FAK inhibitor may be administered or formulated in combination with a RAS(ON) multi-selective inhibitor of the present disclosure and/or any additional therapeutic agent described herein. In some embodiments, a FAK inhibitor is one or more of BI853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii) ROCK Inhibitors

In some embodiments, compositions and methods described herein may include a RAS(ON) multi-selective inhibitor of the present disclosure in combination with one or more Rho-associated, coiled-coil containing protein kinase (ROCK) inhibitors. A ROCK inhibitor may be administered or formulated in combination with a RAS(ON) multi-selective inhibitor of the present disclosure and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN108047193, CN107973777, CN108047197, CN108129448, CN 115869304, and GB202214708, each of which is incorporated herein by reference in its entirety, including the compound structures disclosed therein which are specifically incorporated herein by reference.

### iv) MSK1 Inhibitors

In some embodiments, compositions and methods described herein may include a RAS(ON) multi-selective inhibitor of the present disclosure in combination with one or more Mitogen- and stress-activated kinase (MSK1) inhibitors. A MSK1 inhibitor may be administered or formulated in combination with a RAS(ON) multi-selective inhibitor of the present disclosure and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

### v) RSK Inhibitors

In some embodiments, compositions and methods described herein may include a RAS(ON) multi-selective inhibitor of the present disclosure in combination with one or more ribosomal S6 kinase (RSK) inhibitors. A RSK1 inhibitor may be administered or formulated in combination with a RAS(ON) multi-selective inhibitor of the present disclosure and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

### vi) ALK Inhibitors

In some embodiments, compositions and methods described herein may include a RAS(ON) multi-selective inhibitor of the present disclosure in combination with one or more Anaplastic Lymphoma Kinase (ALK) inhibitors. An ALK inhibitor may be administered or formulated in combination with a RAS(ON) multi-selective inhibitor of the present disclosure and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894. In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN107987056, and CN 105878248, each of which is incorporated herein by reference in its entirety, including the compound structures disclosed therein which are specifically incorporated herein by reference.

### c) Receptor Tyrosine Kinase Inhibitors

Compositions and methods described herein may include a RAS(ON) multi-selective inhibitor of the present disclosure in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

### i) EGFR Inhibitors

In some embodiments, compositions and methods described herein may include a RAS(ON) multi-selective inhibitor of the present disclosure in combination with one or more EGFR inhibitors. An EGFR inhibitor may be administered or formulated in combination with a RAS(ON) multi-selective inhibitor of the present disclosure and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), Lazertinib, erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). In some embodiments, an EGFR inhibitor is one or more of cetuximab, gefitinib (Iressa), erlotinib (Tarceva), and afatinib (Gilotrif). Additional non-limiting examples of small molecule EGFR inhibitors include any of the EGFR inhibitors described in Traxler et al., Exp. Opin. Ther. Patents 1998, 8(12):1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN111973601, CN 111973602, and CN113896744, each of which is incorporated herein by reference in its entirety, including the compound structures disclosed therein which are specifically incorporated herein by reference.

### ii) HER2 Inhibitors

In some embodiments, compositions and methods described herein may include a RAS(ON) multi-selective inhibitor of the present disclosure in combination with one or more HER2 inhibitors. A HER2 inhibitor may be administered or formulated in combination with a RAS(ON) multi-selective inhibitor of the present disclosure and/or any additional therapeutic agent described herein. In some embodiments, an HER2 inhibitor is one or more of tucatinib, rastuzumab (Herceptin™), pertuzumab (Perjeta™), lapatinib (Tykerb™), ado-trastuzumab emtansine (Kadcyla™), and neratinib (Nerlynx™). Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include a RAS(ON) multi-selective inhibitor of the present disclosure in combination with one or more MET inhibitors. A MET inhibitor may be administered or formulated in combination with a RAS(ON) multi-selective inhibitor of the present disclosure and/or any additional therapeutic agent described herein. In some embodiments, a MET inhibitor is one or more of Crizotinib (Xalkori™), Cabozantinib (Cometriq, Cabomety™), Capmatinib (Tabrecta™), Tepotinib (Tepmetko™), Savolitinib (Volitinib™), Onartuzumab (MetMab™), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US 2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include a RAS(ON) multi-selective inhibitor of the present disclosure in combination with one or more AXL inhibitors. An AXL inhibitor may be administered or formulated in combination with a RAS(ON) multi-selective inhibitor of the present disclosure and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include a RAS(ON) multi-selective inhibitor of the present disclosure in combination with one or more insulin-like growth factor receptor 1 (IGF-1R) inhibitors. An IGFR inhibitor may be administered or formulated in combination with a RAS(ON) multi-selective inhibitor of the present disclosure and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### vi) RET Inhibitors

In some embodiments, compositions and methods described herein may include a RAS(ON) multi-selective inhibitor of the present disclosure in combination with one or more Rearranged during transfection (RET) inhibitors. An RET inhibitor may be administered or formulated in combination with a RAS(ON) multi-selective inhibitor of the present disclosure and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN111233899, and CN111362923, each of which is incorporated herein by reference in its entirety, including the compound structures disclosed therein which are specifically incorporated herein by reference.

vii) ROS1 Inhibitors

In some embodiments, compositions and methods described herein may include a RAS(ON) multi-selective inhibitor of the present disclosure in combination with one or more c-ros oncogene 1 (ROS1) inhibitors. A ROS1 inhibitor may be administered or formulated in combination with a RAS(ON) multi-selective inhibitor of the present disclosure and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051 b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US 2017079972, each of which is incorporated herein by reference in its entirety, including the compound structures disclosed therein which are specifically incorporated herein by reference.

viii) PDGFR Inhibitors

In some embodiments, compositions and methods described herein may include a RAS(ON) multi-selective inhibitor of the present disclosure in combination with one or more platelet-derived growth factor receptor (PDGFR) inhibitors. A PDGFR inhibitor may be administered or formulated in combination with a RAS(ON) multi-selective inhibitor of the present disclosure and/or any additional therapeutic agent described herein. PDGFR is a family of receptor tyrosine kinases that consists of two members, PDGFRα and PDGFRβ. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (Ofev™), sunitinib (Sutent™), pazopanib (Votrient™), regorafenib (Stivarga™), and dasatinib (Sprycel™).

### ix) FGF Inhibitors

In some embodiments, compositions and methods described herein may include a RAS(ON) multi-selective inhibitor of the present disclosure in combination with fibroblast growth factor (FGF) inhibitors. An FGF inhibitor may be administered or formulated in combination with a RAS(ON) multi-selective inhibitor of the present disclosure and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (Balversa™), infigratinib (Truseltiq™), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human IgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

### x) VEGF Inhibitors

In some embodiments, compositions and methods described herein may include a RAS(ON) multi-selective inhibitor of the present disclosure in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with a RAS(ON) multi-selective inhibitor of the present disclosure and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### d) PI3K/mTOR Pathway Inhibitors

Compositions and methods described herein may include a RAS(ON) multi-selective inhibitor of the present disclosure in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

### i) PI3K Inhibitors

In some embodiments, compositions and methods described herein may include a RAS(ON) multi-selective inhibitor of the present disclosure in combination with one or more PI3K inhibitors. A PI3K inhibitor may be administered or formulated in combination with a RAS(ON) multi-selective inhibitor of the present disclosure and/or any additional therapeutic agent described herein. A PI3K inhibitor useful in a combination of the disclosure may be a PI3Kα:RAS breaker, such as BBO-1-0203. PI3K inhibitors include, but are not limited to, wortmannin; 17-hydroxywortmannin analogs described in WO 2006044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO 2009036082 and WO 2009055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO 2006122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO 2008070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib. In some embodiments, reference to the term PI3K inhibitor includes any such PI3K inhibitor disclosed in any one of the following patent applications WO 2025072451 A1, WO 2025061125 A1, WO 2025051235 A1, WO 2025045106 A1, WO 2025040167 A1, WO 2025036439 A1, WO 2025038698 A1, WO 2025038395 A1, WO 2025034858 A1, WO 2025034849 A1, WO 2025029683 A1, WO 2025016314 A1, WO 2025003330 A1, WO 2025007074 A1, WO 2025002179 A1, WO 2024260464 A1, WO 2024229121 A1, WO 2024222894 A1, WO 2024215799 A1, WO 2024192309 A1, WO 2024183806 A1, WO 2024182404 A1, WO 2024182447 A1, each of which is incorporated herein by reference in its entirety.

### ii) AKT Inhibitors

In some embodiments, compositions and methods described herein may include a RAS(ON) multi-selective inhibitor of the present disclosure in combination with one or more AKT inhibitors. An AKT inhibitor may be administered or formulated in combination with a RAS(ON) multi-selective inhibitor of the present disclosure and/or any additional therapeutic agent described herein. AKT inhibitors include, but are not limited to, ipatasertib, GSK-2141795, Akt-1-1 (inhibits AktI) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii) mTOR Inhibitors

In some embodiments, compositions and methods described herein may include a RAS(ON) multi-selective inhibitor of the present disclosure in combination with one or more mTOR inhibitors. A mTOR inhibitor may be administered or formulated in combination with a RAS(ON) multi-selective inhibitor of the present disclosure and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO 199409010); ridaforolimus (also known as deforolimus or AP23573); rapalogs, e.g., as disclosed in WO 199802441 and WO 200114387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO 2005005434; derivatives disclosed in U.S. Pat. Nos. 5,258,389, 5,118,677, 5,118,678, 5,100,883, 5,151,413, 5,120,842, and 5,256,790, and in WO 1994090101, WO 199205179, WO 1993111130, WO 199402136, WO 199402485, WO 199514023, WO 199402136, WO 199516691, WO 199641807, WO 199641807, and WO 2018204416; and phosphorus-containing rapamycin derivatives (e.g., WO 2005016252). In some embodiments, the mTOR inhibitor is a bisteric inhibitor (see, e.g., WO 2018204416, WO 2019212990 and WO 2019212991), such as RMC-5552.

### iv) MNK Inhibitors

In some embodiments, compositions and methods described herein may include a RAS(ON) multi-selective inhibitor of the present disclosure in combination with one or more mitogen-activated protein kinase-interacting kinase (MNK) inhibitors. A MNK inhibitor may be administered or formulated in combination with a RAS(ON) multi-selective inhibitor of the present disclosure and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN115583942, each of which is incorporated herein by reference in its entirety, including the compound structures disclosed therein which are specifically incorporated herein by reference.

v) eIF4 Inhibitors

In some embodiments, compositions and methods described herein may include a RAS(ON) multi-selective inhibitor of the present disclosure in combination with one or more eukaryotic initiation factor 4A (eIF4A) inhibitors. An eIF4A inhibitor may be administered or formulated in combination with a RAS(ON) multi-selective inhibitor of the present disclosure and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with a RAS(ON) multi-selective inhibitor of the present disclosure and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### e) DNA Damage Response Inhibitors

Compositions and methods described herein may include a RAS(ON) multi-selective inhibitor of the present disclosure in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

### i) Wee1 Inhibitors

In some embodiments, compositions and methods described herein may include one or more Wee1 inhibitors. A Wee1 inhibitor may be administered or formulated in combination with a RAS(ON) multi-selective inhibitor of the disclosure and/or any additional therapeutic agent described herein. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

### ii) CHK Inhibitors

In some embodiments, compositions methods described herein may include one or more checkpoint kinase (CHK) inhibitors. A CHK inhibitor may be administered or formulated in combination with a RAS(ON) multi-selective inhibitor of the disclosure and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more BBI-355, rabusertib, LY2606368, LY2880070, GDC-0575, MK-8776, BEBT-260, and PEP07. In some embodiments, reference to the term CHK1 inhibitor includes any such CHK1 inhibitor disclosed in any one of the following patent applications: WO 2024196923, WO 2024211271, WO 2024211270, WO 2024118564, WO 2023230477, WO 2022251502, WO 2021113661, WO 2021104461, WO 2019012030, WO 2010118390, WO 2008067027, WO 2002070494, CN119661557, and TW202126818, each of which is incorporated herein by reference in its entirety, including the compound structures disclosed therein which are specifically incorporated herein by reference.

iii) ATM Inhibitors

In some embodiments, compositions and methods described herein may include one or more ataxia telangiectasia mutated (ATM) inhibitors. An ATM inhibitor may be administered or formulated in combination with a RAS(ON) multi-selective inhibitor of the disclosure and/or any additional therapeutic agent described herein. ATM plays a role in regulating the replication stress response and maintaining genomic stability. In some embodiments, an ATM inhibitor is one or more lartesertib, AZD1390, AZD0156, KU-60019, M4076, M3541, WSD-0628, ZN-B-2262, SYH2051, and VE-821. In some embodiments, reference to the term ATM inhibitor includes any such ATM inhibitor disclosed in any one of the following patent applications: WO 2024189299, WO 2022058351, WO 2021197339, WO 2021098734, WO 2021260580, WO 2020193660, WO 2020063855, WO 2016155884, WO 2007026157, WO 2006085067, US 2016113935, CN 116440082, CN 117180432 and CN 115105596 each of which is incorporated herein by reference in its entirety, including the compound structures disclosed therein which are specifically incorporated herein by reference.

### iv) ATR Inhibitors

In some embodiments, compositions and methods described herein may include one or more ataxia telangiectasia and Rad3-related (ATR) inhibitors. An ATR inhibitor may be administered or formulated in combination with a RAS(ON) multi-selective inhibitor of the disclosure and/or any additional therapeutic agent described herein. In some embodiments, an ATR inhibitor is one or more berzosertib, gartisertib, camonsertib, ceralaertib, VE-821, RP-3500, AZ20, VX-970, abd110, VX-803, and elimusertib (BAY 1895344). In some embodiments, reference to the term ATR inhibitor includes any such ATR inhibitor disclosed in any one of the following patent applications: WO 2025019344, WO 2025019346, WO 2023138343, WO 2023126823, WO 2023109883, WO 2023016529, WO 2022237875, WO 2022268025, WO 2021012049, WO 2021023272, WO 2021260579, WO 2021228758, WO 2019050889, WO 2019154365, WO 2019036641, WO 2019133711, WO 2017059357, WO 2013049859, WO 2007046426, WO 2007015632, and CN113797341, each of which is incorporated herein by reference in its entirety, including the compound structures disclosed therein which are specifically incorporated herein by reference.

### v) PARP Inhibitors

In some embodiments, compositions and methods described herein may include one or more Poly(ADP-ribose) polymerase (PARP) inhibitors. A PARP inhibitor may be administered or formulated in combination with a RAS(ON) multi-selective inhibitor of the disclosure and/or any additional therapeutic agent described herein. There are 17 PARP (aka tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more olaparib, rucaparib, niraparib, and veliparib (ABT-888). In some embodiments, reference to the term PARP inhibitor includes any such PARP inhibitor disclosed in any one of the following patent applications: WO 2025024581, WO 2025037273, WO 2025061057, WO 2024256377, WO 2024255782, WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN113698356, CN 113603647, CN 115073544, CN 108938634, CN 104887680, CN 110343088, CN108976236, CN 117069731, CN 119185316, CN 119112794, and CN 107629071, each of which is incorporated herein by reference in its entirety, including the compound structures disclosed therein which are specifically incorporated herein by reference.

### vi) DNA-PK Inhibitors

In some embodiments, compositions and methods described herein may include one or more DNA-dependent protein kinase (DNA-PK) inhibitors. A DNA-PK inhibitor may be administered or formulated in combination with a RAS(ON) multi-selective inhibitor of the disclosure and/or any additional therapeutic agent described herein. DNA-PK is a serine/threonine protein kinase that plays a crucial role in DNA repair and maintenance of genome stability. In some embodiments, a DNA-PK inhibitor is one or more NU7441, AZD7648, VX-984, peposertib (M3814), and CC-115. In some embodiments, reference to the term DNA-PK inhibitor includes any such DNA-PK inhibitor disclosed in any one of the following patent applications: WO 2025023957, WO 2023220418, WO 2023215991, WO 2023165603, WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, CN 115322209, and CN 112300126, each of which is incorporated herein by reference in its entirety, including the compound structures disclosed therein which are specifically incorporated herein by reference.

### f) Cell Cycle Inhibitors

Compositions and methods described herein may include a RAS(ON) multi-selective inhibitor of the present disclosure in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

### i) CDK Inhibitors

In certain embodiments, a cell cycle inhibitor is a cyclin-dependent kinase (CDK) inhibitor. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib (P1446A-05), BLU-222, dinaciclib, AT-7519, RGB286638, and AZD4573.

In some embodiments, reference to the term CDK inhibitor includes any such CDK inhibitor disclosed in any one of the following patent applications: WO 2025040170, WO 2025060620, WO 2024238574, WO 2024027825, WO 2024048541, WO 2022166793, WO 2022187611, WO 2022130304, WO 2021227906, WO 2021057867, WO 2020207260, WO 2020138370, WO 2020125513, WO 2020093011, WO 2020148635, WO 2020215156, WO 2020052627, WO 2017177837, WO 2017162215, WO 2017177836, WO 2017172826, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN107652284, KR 20180106188, and US 2017152269, each of which is incorporated herein by reference in its entirety, including the compound structures disclosed therein which are specifically incorporated herein by reference.

### ii) Aurora Kinase Inhibitors

In some embodiments, compositions and methods described herein may include a RAS(ON) multi-selective inhibitor of the present disclosure in combination with one or more aurora kinase inhibitors. An aurora kinase inhibitor may be administered or formulated in combination with a RAS(ON) multi-selective inhibitor of the present disclosure and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii) PLK Inhibitors

In some embodiments, compositions and methods described herein may include a RAS(ON) multi-selective inhibitor of the present disclosure in combination with one or more polo-like kinase (PLK) inhibitors. A PLK inhibitor may be administered or formulated in combination with a RAS(ON) multi-selective inhibitor of the present disclosure and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

### iv) Kinesin Superfamily of Microtubule Motor Protein Inhibitors

In some embodiments, compositions and methods described herein may include a RAS(ON) multi-selective inhibitor of the present disclosure in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with a RAS(ON) multi-selective inhibitor of the present disclosure and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

### v) DYRK1 Inhibitors

In some embodiments, compositions and methods described herein may include a RAS(ON) multi-selective inhibitor of the present disclosure in combination with one or more Dual-specificity tyrosine phosphorylation-regulated kinase 1 (DYRK1) inhibitors. A DYRK1 inhibitor may be administered or formulated in combination with a RAS(ON) multi-selective inhibitor of the present disclosure and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### g) Anti-Apoptotic Protein Inhibitors

Compositions and methods described herein may include a RAS(ON) multi-selective inhibitor of the present disclosure in combination with one or more anti-apoptotic protein inhibitors. In some embodiments, an anti-apoptotic protein inhibitor may be administered or formulated in combination with a RAS(ON) multi-selective inhibitor of the present disclosure and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a RAS(ON) multi-selective inhibitor of the disclosure and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### h) Autophagy Inhibitors

Compositions and methods described herein may include a RAS(ON) multi-selective inhibitor of the present disclosure in combination with one or more autophagy inhibitors. In some embodiments, an autophagy inhibitor may be administered or formulated in combination with a RAS(ON) multi-selective inhibitor of the present disclosure and/or any additional therapeutic agent described herein. Autophagy inhibitors include, but are not limited to chloroquine, 3-methyladenine, hydroxychloroquine (Plaquenil™) spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### a) ULK Inhibitors

In some embodiments, compositions and methods described herein may include a RAS(ON) multi-selective inhibitor of the present disclosure in combination with one or more Unc-51-like kinase (ULK) inhibitors. An ULK inhibitor may be administered or formulated in combination with a RAS(ON) multi-selective inhibitor of the present disclosure and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### b) VPS Inhibitors

In some embodiments, compositions and methods described herein may include a RAS(ON) multi-selective inhibitor of the present disclosure in combination with one or more Vacuolar protein sorting protein (VPS) inhibitors. A VPS inhibitor may be administered or formulated in combination with a RAS(ON) multi-selective inhibitor of the present disclosure and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK-III, VPS34-IN1, SAR405, Spautin-1, and NSC185058.

### c) Macropinocytosis Inhibitors

In some embodiments, compositions and methods described herein may include a RAS(ON) multi-selective inhibitor of the present disclosure in combination with one or more macropinocytosis inhibitors. A macropinocytosis inhibitor may be administered or formulated in combination with a RAS(ON) multi-selective inhibitor of the disclosure and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

### i) WNT/β-Catenin Pathway Inhibitors

Compositions and methods described herein may include a RAS(ON) multi-selective inhibitor of the present disclosure in combination with one or more WNT/beta-catenin pathway inhibitors. In some embodiments, a WNT/beta-catenin pathway inhibitor may be administered or formulated in combination with a RAS(ON) multi-selective inhibitor of the present disclosure and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

### i) β-Catenin Inhibitors

In some embodiments, compositions and methods described herein may include a RAS(ON) multi-selective inhibitor of the present disclosure and one or more β-catenin inhibitors. A β-catenin inhibitor may be administered or formulated in combination with a RAS(ON) multi-selective inhibitor of the present disclosure and/or any additional therapeutic agent described herein. Beta-catenin is a protein that plays an important role in the WNT signaling pathway, which regulates various cellular processes including cell proliferation, differentiation, and migration. In normal cells, β-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/(3-catenin inhibitor is one or more of FOG-001, OMP-131R10, Foxy-5, LGK974, RXC004, ETC-159, OMP-54F28, Niclosamide, OMP-18R5, OTSA-101, BNC101, DKN-01, Sulindac, Pyrvinium, E7449, BC2059, PRI-724, SM08502, IWP1, IWP2, IWP3, IWP4, IWP12, IWP L6, C59, GNF-6231, GNF-1331, DK-520, DK-419, IgG-2919, Fz7-21, RHPD-P1, SR137892, 1094-0205, 2124-0331, 3235-0367, NSC36784, NSC654259, IgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivant, foscenvivant, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term β-catenin inhibitor includes any such β-catenin inhibitor disclosed in any one of the following patent applications: CN 104388427 and CN 103830211, each of which is incorporated herein by reference in its entirety, including the compound structures disclosed therein which are specifically incorporated herein by reference.

### ii) PORCN Inhibitors

In some embodiments, compositions and methods described herein may include a RAS(ON) multi-selective inhibitor of the present disclosure in combination with one or more Porcupine (PORCN) inhibitors. A PORCN inhibitor may be administered or formulated in combination with a RAS(ON) multi-selective inhibitor of the present disclosure and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii) GSK3 Inhibitors

In some embodiments, compositions and methods described herein may include a RAS(ON) multi-selective inhibitor of the present disclosure in combination with one or more Glycogen synthase kinase (GSK3) inhibitors. A GSK3 inhibitor may be administered or formulated in combination with a RAS(ON) multi-selective inhibitor of the present disclosure and/or any additional therapeutic agent described herein. The GSK3 family consists of two closely related serine/threonine kinases: GSK3a and GSK3p. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, laduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

### iv) CLK Inhibitors

In some embodiments, compositions and methods described herein may include a RAS(ON) multi-selective inhibitor of the present disclosure in combination with one or more Cdc2-like kinase (CLK) inhibitors. A CLK inhibitor may be administered or formulated in combination with a RAS(ON) multi-selective inhibitor of the present disclosure and/or any additional therapeutic agent described herein. LKs (Cdc2-like kinases) are a family of serine/threonine kinases that play a crucial role in pre-mRNA splicing, specifically in the regulation of alternative splicing. There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH-CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### j) JAK/STAT Pathway Inhibitors

Compositions and methods described herein may include a RAS(ON) multi-selective inhibitor of the present disclosure in combination with one or more JAK/STAT pathway inhibitors. In some embodiments, a JAK/STAT pathway inhibitor may be administered or formulated in combination with a RAS(ON) multi-selective inhibitor of the present disclosure and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (Jakafi®), Pacritinib, Fedratinib, Tofacitinib (Xeljanz®), Abrocitinib, Filgotinib, Oclacitinib, Peficitinib, Upadacitinib, Deucravacitinib, Delgocitinib, and Baricitinib (Olumiant®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT3 degrader is KT-333. In some embodiments, the STAT inhibitor is one or more of TTI-101, C-188-9, WP1066, VVD-130850, LLL12B, STA-21, SD-36, Stattic, S31-201, OPB-31121, KT-333, and Napabucasin (BB1608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### k) Epigenetic Modulators

Compositions and methods described herein may include a RAS(ON) multi-selective inhibitor of the present disclosure in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

### i) HDAC Inhibitors

In some embodiments, compositions and methods described herein may include a RAS(ON) multi-selective inhibitor of the present disclosure in combination with one or more histone deacetylase (HDAC) inhibitors. A HDAC inhibitor may be administered or formulated in combination with a RAS(ON) multi-selective inhibitor of the present disclosure and/or any additional therapeutic agent described herein. There are several classes of HDACs, including class I, class Ila, class IIb, class III, and class IV. Class I HDACs are further divided into HDAC1, HDAC2, HDAC3, and HDAC8, while class Ila HDACs include HDAC4, HDAC5, HDAC7, and HDAC9. Class IIb HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (Zolinza™), Romidepsin (Istodax™), Belinostat (Beleodaq™), Panobinostat (Farydak™), Entinostat (MS-275), Valproic acid (Depakene™), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

### ii) BET Inhibitors

In some embodiments, compositions and methods described herein may include a RAS(ON) multi-selective inhibitor of the present disclosure in combination with one or more bromodomain and extra-terminal protein (BET) inhibitors. A BET inhibitor may be administered or formulated in combination with a RAS(ON) multi-selective inhibitor of the present disclosure and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii) EZH2 Inhibitors

In some embodiments, compositions and methods described herein may include a RAS(ON) multi-selective inhibitor of the present disclosure in combination with one or more Enhancer of Zeste Homolog 2 (EZH2) inhibitors. An EZH2 inhibitor may be administered or formulated in combination with a RAS(ON) multi-selective inhibitor of the present disclosure and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

### iv) Co-REST Inhibitors

In some embodiments, compositions and methods described herein may include a RAS(ON) multi-selective inhibitor of the present disclosure in combination with one or more Co-REST inhibitors. A Co-REST inhibitor may be administered or formulated in combination with a RAS(ON) multi-selective inhibitor of the present disclosure and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

### v) EP300

In some embodiments, compositions and methods described herein may include a RAS(ON) multi-selective inhibitor of the present disclosure in combination with one or more E1A-binding protein p300 (EP300) inhibitors. An EP300 inhibitor may be administered or formulated in combination with a RAS(ON) multi-selective inhibitor of the present disclosure and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 inhibitor is one or more of C646, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

### vi) LSD1

In some embodiments, compositions and methods described herein may include a RAS(ON) multi-selective inhibitor of the present disclosure in combination with one or more Lysine-specific demethylase 1 (LSD1) inhibitors. A LSD1 inhibitor may be administered or formulated in combination with a RAS(ON) multi-selective inhibitor of the present disclosure and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN107174584, CN 110478352, CN 106432248, and CN 106045881, each of which is incorporated herein by reference in its entirety, including the compound structures disclosed therein which are specifically incorporated herein by reference.

vii) PRMT5

In some embodiments, compositions and methods described herein may include a RAS(ON) multi-selective inhibitor of the present disclosure in combination with one or more Protein arginine methyltransferase 5 (PRMT5) inhibitors. A PRMT5 inhibitor may be administered or formulated in combination with a RAS(ON) multi-selective inhibitor of the present disclosure and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii) MAT2A

In some embodiments, compositions and methods described herein may include a RAS(ON) multi-selective inhibitor of the present disclosure in combination with one or more methionine adenosyltransferase 2A (MAT2A) inhibitors. A MAT2A inhibitor may be administered or formulated in combination with a RAS(ON) multi-selective inhibitor of the present disclosure and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

### ix) DOT1L

In some embodiments, compositions and methods described herein may include a RAS(ON) multi-selective inhibitor of the present disclosure in combination with one or more Disruptor of Telomeric silencing 1-like (DOT1L) inhibitors. A DOT1L inhibitor may be administered or formulated in combination with a RAS(ON) multi-selective inhibitor of the present disclosure and/or any additional therapeutic agent described herein. DOT1L is a histone methyltransferase enzyme that catalyzes the methylation of lysine 79 on histone H3. This modification is associated with transcriptional elongation and is important for the maintenance of gene expression programs. The DOT1L family includes enzymes that are involved in epigenetic regulation and transcriptional control, and their dysregulation has been linked to various diseases, including cancer. In some embodiments, a DOT1L inhibitor is one or more of EPZ-5676 (pinometostat) and EPZ-004777. In some embodiments, reference to the term DOT1L inhibitor includes any such DOT1L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

iix) UBA1

In some embodiments, compositions and methods described herein may include a RAS(ON) multi-selective inhibitor of the present disclosure in combination with one or more ubiquitin-activating enzyme inhibitors (e.g., a UBA1 inhibitor). A UBA1 inhibitor may be administered or formulated in combination with a RAS(ON) multi-selective inhibitor of the present disclosure and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### l) Ribonucleotide Reductase Inhibitors

Compositions and methods described herein may include a RAS(ON) multi-selective inhibitor of the present disclosure in combination with one or more ribonucleotide reductase inhibitors (RNRi). RNR inhibitors are a class of compounds that inhibit the enzyme ribonucleotide reductase, which is essential for DNA synthesis and repair. RNR catalyzes the conversion of ribonucleotides (RNA building blocks) into deoxyribonucleotides (DNA building blocks), providing the necessary precursors for DNA replication and repair in proliferating cells. By inhibiting RNR, these compounds effectively limit the production of deoxyribonucleotides, thereby preventing DNA synthesis and halting the proliferation of rapidly dividing cells, such as cancer cells.

RNR is composed of two subunits: the R1 large subunit (containing the catalytic site) and the R2 small subunit (containing a di-iron center critical for enzymatic activity). RRIs typically act by binding to either the active site on the R1 subunit or the iron-oxygen complex in the R2 subunit, leading to the inhibition of the enzyme's activity. In some embodiments, a RNR inhibitor is a nucleoside analog inhibitor, an iron chelator, or an allosteric inhibitor. In some embodiments, a RNR inhibitor useful according to the present disclosure include but are not limited to one or more of hydroxyurea, triapine, didox, GTI-2040, CPI-613 (devimistat), and clofarabine. In some embodiments, reference to the term RNR inhibitor includes any such RNR inhibitor disclosed in any one of the following patent applications: WO 2025049814, WO 2022059691, WO 2022059692, WO 2021034776, WO 2019106579, WO 2014205179, WO 2013105088, WO 199312782, U.S. Pat. Nos. 5,071,835, 5,405,850, 4,814,432, and WO 199518815 each of which is incorporated herein by reference in its entirety, including the compound structures disclosed therein which are specifically incorporated herein by reference.

### m) Additional Therapeutic Agents Useful for Combination Therapy

In some embodiments, compositions and methods described herein may include a RAS(ON) multi-selective inhibitor of the present disclosure in combination with one or more Farnesyl transferase inhibitors. A farnesyl transferase inhibitor may be administered or formulated in combination with a RAS(ON) multi-selective inhibitor of the present disclosure and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapiadib. In some embodiments, reference to the term farnesyl transferase inhibitor includes any such farnesyl transferase inhibitor disclosed in any one of the following patent applications: WO 2010057028, WO 2007042465, WO 200136395, WO 200064891, WO 200042849, WO 199938862, WO 199928315, WO199829390, WO 199426723, CN 107312000, CN 107365310, KR 100375421, KR 100388790, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more casein kinase inhibitors.

In some embodiments, a casein inhibitor is, SR-3029, a potent and ATP competitive CK1δ and CK1ε inhibitor.

In some embodiments, compositions and methods described herein may include one or more FLT3 inhibitors in combination with a RAS(ON) multi-selective inhibitor of the present disclosure disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, ponatinib and quizartinib.

In some embodiments, compositions and methods described herein may include a RAS(ON) multi-selective inhibitor of the present disclosure in combination with one or more one or more TGFβ pathway inhibitors. In some embodiments, compositions and methods described herein may include one or more TGFβ inhibitors. A TGFβ inhibitor may be administered or formulated in combination with a RAS(ON) multi-selective inhibitor of the present disclosure and/or any additional therapeutic agent described herein. TGFβ (transforming growth factor beta) is a multifunctional cytokine involved in various cellular processes, including cell growth, differentiation, apoptosis, and immune response. Dysregulation of the TGFβ signaling pathway has been implicated in various diseases, including cancer, fibrosis, and autoimmune disorders. In some embodiments, a TGFβ inhibitor is one or more of galunisertib (LY2157299), and vactosertib (TEW-7197). In some embodiments, a TGFβ inhibitor is one or more of Galunisertib, LY2157299, Fresolimumab, Lerdelimumab, Trabedersen, curcumin, resveratrol and small interfering RNA (siRNA) to silence TGFβ receptor expression. In some embodiments, reference to the term TGFβ inhibitor includes any such TGFβ inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more HSP90 inhibitors. A HSP90 inhibitor may be administered or formulated in combination with a RAS(ON) multi-selective inhibitor of the present disclosure and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU-H71), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more Glutathione peroxidase 4 (GPX4) inhibitors. A GPX4 inhibitor may be administered or formulated in combination with a RAS(ON) multi-selective inhibitor of the present disclosure and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US 2021244715, and KR 20220115536, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more NRF2 inhibitors. A NRF2 inhibitor may be administered or formulated in combination with a RAS(ON) multi-selective inhibitor of the present disclosure and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more TEA domain (TEAD) inhibitors. A TEAD inhibitor may be administered or formulated in combination with a RAS(ON) multi-selective inhibitor of the present disclosure and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT3989, VT-107, a pan-TEAD, VT-104, Verteporfin, CA3, IAG933, K-975, IK-595, and Statins (see, e.g., Chapeau, Emilie and Schmelzle, Tobias (2023) IAG933, an oral selective YAP1-TAZ/pan-TEAD protein-protein interaction inhibitor (PPli) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more NOTCH/Gamma secretase inhibitors. A NOTCH/Gamma secretase inhibitor may be administered or formulated in combination with a RAS(ON) multi-selective inhibitor of the present disclosure and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include a RAS(ON) multi-selective inhibitor of the present disclosure in combination with one or more Hedgehog inhibitors. A hedgehog inhibitor may be administered or formulated in combination with A RAS(ON) multi-selective inhibitor of the present disclosure and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (Erivedge), Sonidegib (Odomzo), and Glasdegib (Daurismo). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include a RAS(ON) multi-selective inhibitor of the present disclosure in combination with one or more NFkB pathway inhibitors. An NFkB inhibitor may be administered or formulated in combination with a RAS(ON) multi-selective inhibitor of the present disclosure and/or any additional therapeutic agent described herein. NF-kappa B (NFκB) is a family of transcription factors involved in regulating various cellular processes, including inflammation, immunity, cell survival, and proliferation. Non-limiting examples of NFkB inhibitors include Bortezomib (Velcade), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, a RAS(ON) multi-selective inhibitor of the present disclosure can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, a RAS(ON) multi-selective inhibitor of the present disclosure may be used as an adjuvant therapy after surgery. In some embodiments, a RAS(ON) multi-selective inhibitor of the present disclosure may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, a RAS(ON) multi-selective inhibitor of the present disclosure can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a RAS(ON) multi-selective inhibitor of the present disclosure, which amount is effective to sensitize abnormal cells to treatment with radiation. The amount of the compound in this method can be determined according to the means for ascertaining effective amounts of such compounds described herein. In some embodiments, a RAS(ON) multi-selective inhibitor of the present disclosure may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, compositions and methods described herein may include a RAS(ON) multi-selective inhibitor of the present disclosure in combination with one or more Claudin-18 targeting agents. A Claudin-18 targeting agents may be administered or formulated in combination with a RAS(ON) multi-selective inhibitor of the present disclosure and/or any additional therapeutic agent described herein. Claudin-18 (e.g., claudin 18.2; CLDN18.2) has become a promising target for the treatment of patients with digestive malignancies, such as gastric cancer (GC), gastroesophageal junction (GEJ) cancer, esophageal cancer, and pancreatic cancer, because of its limited expression in healthy tissues and abnormal overexpression in a range of malignancies. Multiple clinical trials of CLDN18.2-targeted therapies, including monoclonal antibodies, bispecific antibodies, antibody-drug conjugates (ADCs), and chimeric antigen receptor (CAR) T-cell therapies, are ongoing, with some showing promising early results. Malignant transformation of gastric epithelial tissue leads to disruption of cell polarity and then to exposure of CLDN18.2 epitopes on the cell surface. Although targeted monoclonal antibodies are largely unable to access CLDN18.2 located in tight-junction supramolecular complexes in normal tissue, the perturbations in cell polarity that expose CLDN18.2 epitopes may theoretically enable CLDN18.2 targeted agents to bind to CLDN18.2 in malignant tissues with minimal off-target effects, making CLDN18.2 an attractive target for therapy. In some embodiments, a Claudin-18 targeting agent is one or more of Zolbetuximab, ASKB589, Osemitamab (TST001), PT886 (a bispecific antibody that targets CLDN18.2 and CD47), TJ-CD4B, CMG901 (an ADC that is composed of an antiCLDN18.2 monoclonal antibody joined to a cytotoxic payload, monomethyl auristatin E), and CTO41 (autologous T cells genetically engineered to express a CLDN18.2-targeted CAR). In some embodiments, reference to the term Claudin-18 targeting agent includes any such Claudin-18 targeting agent disclosed in any one of the following patent applications: WO 2024081544, WO 2024131683, WO 2024137619, WO 2024140670, WO 2024136594, WO 2023034922, WO 2023046202, WO 2022203090, WO 2022133169, WO 2022100613, WO 2022256449, WO 2022136642, WO 2021155380, WO 2021129765, WO 2021011885, WO 2021058000, WO 2021218874, WO 2021027850, WO 2020156554, WO 2020025792, WO 2020114480, WO 2020211792, WO 2020239005, WO 2019219089, WO 2018157147, WO 2018108106, WO 2016166122, WO 2014146778, CN 118290582, CN118203658, and CN 118286201, each of which is incorporated herein by reference in its entirety, including the compound structures disclosed therein which are specifically incorporated herein by reference.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a RAS(ON) multi-selective inhibitor of the present disclosure include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO 200137820, WO 200132651, WO 200268406, WO 200266470, WO 200255501, WO 200405279, WO 200407481, WO 200407458, WO 200409784, WO 200259110, WO 199945009, WO 2000/59509, WO 199961422, WO 200012089, and WO 200002871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, A2bR, A2aR/A2bR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MED10680, BMS936559, MED14736, MPDL3280A, MSB0010718C, BMS986016, IMP321, lirilumab, IPH2101, 1-7F9, and KW-6002. Non-limiting examples of immune modulatory agent includes targets identified in Table 2.

TABLE 2
Exemplary Immune Modulatory Targets

| Target | Biological Function | Target | Biological Function |
|---|---|---|---|
| CTLA-4 | Inhibitory Receptor | A2aR, A2bR or | Inhibitory Receptor |
| | | both A2aR/A2bR | |
| PD-1 | Inhibitory Receptor | CD73 | Inhibitory Receptor |
| PD-L1 | Ligand for PD-1 | CD39 | Inhibitory Receptor |
| LAG-3 | Inhibitory Receptor | PVRIG | Inhibitory Receptor |
| B7.1 | Costimulatory | IDO | Inhibitory enzyme |
| | Molecule | | |
| B7-H3 | Inhibitory Ligand | CSF1R | Inhibitory Receptor |
| B7-H4 | Inhibitory Ligand | LIF | Inhibitory Cytokine |
| TIM3 | Inhibitory Receptor | CD47 | Inhibitory Receptor |
| VISTA | Inhibitory Receptor | SIRPa | Inhibitory Receptor |
| CD137 | Costimulatory | IL-2 | Effector Cytokines |
| | Molecule | | |
| OX-40 | Costimulatory | IL-15 | Effector Cytokines |
| | Receptor | | |
| CD40 | Costimulatory | IL-12 | Effector Cytokines |
| agonist | Molecule | | |
| CD40 | Costimulatory | TREM2 | Receptor |
| agonist + | Molecule | | |
| FLT3 | | | |
| ligand | | | |
| CD27 | Costimulatory | TGFb | Multifunctional |
| | Receptor | | Cytokine |
| CCR4 | Costimulatory | CD73/TGFb | Multifunctional |
| | Receptor | trap | Cytokine |
| GITR | Costimulatory | TCR-T cells | Cell therapy |
| | Receptor | directed to | |
| | | KRAS^{MUT}, | |
| | | mesothelin, or | |
| | | PRAME | |
| NKG2D | Activating Receptor | mRNA cancer | vaccines |
| | | vaccines | |
| KIR | Costimulatory | BiTEs | Bi-specific T-cell |
| | Receptor | | engager |
| NKG2A | Inhibitory Receptor | Dual EP2/EP4 | E-prostanoid |
| | | inhibitor | receptor |
| ENPP1 | Inhibitory Receptor | Gamma delta T | Cell therapy |
| | | Cells | |
| TIGIT | Inhibitory Receptor | NK cells | Cell therapy |

CTLA4, cytotoxic T-lymphocyte-associated antigen 4;
LAG3, lymphocyte activation gene 3;
PD-1, programmed cell death protein 1;
PD-L1, PD-1 ligand;
TIM3, T cell membrane protein 3;
VISTA, V-domain immunoglobulin (Ig)-containing suppressor of T-cell activation;
KIR, killer IgG-like receptor, APC (Antigen Presenting Cells);
TREM2 (Triggering receptor expressed on myeloid cells 2);
TGF-b (Transforming growth factor beta)

In some embodiments, compositions and methods described herein may include a RAS(ON) multi-selective inhibitor of the disclosure in combination with one or more immune checkpoint inhibitor (ICI). An immune checkpoint inhibitor may be administered or formulated in combination with a compound as described herein.

Immune checkpoints refer to a plethora of inhibitory pathways hardwired into the immune system, which, under normal physiological conditions are crucial for maintaining self-tolerance and modulating the duration and amplitude of physiological immune responses in peripheral tissues to minimize collateral tissue damage in response to pathogenic infection. However, the expression of immune checkpoint proteins is often dysregulated by tumors as an important immune resistance and escape mechanism.

Because many of the immune checkpoints are initiated by ligand-receptor interactions, they can be readily blocked by antibodies or modulated by recombinant forms of ligands or receptors. Thus, inhibition of these pathways has been used to activate therapeutic anti-tumor immunity. For example, cytotoxic T-lymph ocyte-associated antigen 4 (CTLA-4) antibodies were the first of this class of immunotherapeutics to achieve US Food and Drug Administration (FDA) approval. Preliminary clinical findings with inhibitors of additional immune-checkpoint proteins, such as programmed cell death protein 1 (PD-1), indicate broad and diverse opportunities to enhance anti-tumor immunity with the potential to produce durable clinical responses.

T cell activation through blockade of immune checkpoints has been a major focus of efforts to therapeutically manipulate endogenous anti-tumor immunity, owing to the capacity of T cells for the selective recognition of peptides derived from proteins in all cellular compartments; their capacity to directly recognize and kill antigen-expressing cells (by CD8+ effector T cells; also known as cytotoxic T lymphocytes (CTLs)); and their ability to orchestrate diverse immune responses (by CD4+ helper T cells), which integrate adaptive and innate effector mechanisms. Thus, agonists of co-stimulatory receptors or antagonists of inhibitory signals, both of which result in the amplification of antigen-specific T cell responses, are currently agents of interest in clinical testing.

ICIs approved or in development include, but are not limited to, YERVOY® (ipilimumab), OPDIVO® (nivolumab), KEYTRUDA® (pembrolizumab), tremelimumab, galiximab, MDX-1106, BMS-936558, MED14736, MPDL3280A, MED16469, BMS-986016, BMS-663513, PF-05082566, IPH2101, KW-0761, CDX-1127, CP-870, CP-893, GSK2831781, MSB0010718C, MK3475, CT-011, AMP-224, MDX-1105, IMP321, and MGA271, as well as numerous other antibodies or fusion proteins directed to the immune checkpoint proteins noted in Table 2. Common immune checkpoint proteins that may be targeted by ICIs include, but are not limited to B7.1, B7-H3, LAG3, CD137, KIR, CCR4, CD27, OX40, GITR, CD40, CTLA4, PD-1, and PD-L1. In some embodiments, the immune checkpoint inhibitor is an inhibitor of a target selected from the group comprising or consisting of programmed cell death protein-1, ligand of PD-1, cytotoxic T-lymphocyte-associated protein 4 (CTLA-4), T cell immunoglobulin and mucin-domain containing-3 (TIM-3), V-domain Ig suppressor of T cell activation (VISTA), lymphocyte-activation gene 3 (LAG-3), T cell immunoglobulin and ITIM domain (TIGIT), B7 homolog 3 protein (B7-H3), B- and T-lymphocyte attenuator (BTLA), Sialic acid binding Ig-like lectin 15 (Siglec-15), cytokine-inducible SH2-containing protein (CISH), and combination thereof.

In some embodiments, the ICI therapy is selected from one or more of anti-PD-1, anti-PD-L1, anti-CTLA-4, anti-LAG3, anti-B7.1, anti-B7H3, anti-B7H4, anti-TIM3, anti-VISTA, anti-CD137, anti-OX40, anti-CD40, anti-CD27, anti-CCR4, anti-GITR, anti-NKG2D, and anti-KIR. In some embodiments, the ICI therapy is an antibody (e.g., a monoclonal antibody selective for any of the targets in Table 2). In some embodiments the ICI is an anti-PD-1 antibody. The antibody may be, e.g., humanized or fully human.

In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (also known as tislelizumab; BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MED10680, BMS936559, MED14736, MPDL3280A, MSB0010718C, BMS986016, IMP321, lirilumab, IPH2101, 1-7F9, and KW-6002.

In some embodiments, the immune checkpoint inhibitor is an inhibitor of programmed cell death protein-1 (PD-1) or an inhibitor of the ligand of PD-1 (PDL-1).

Programmed cell death protein-1 is herein interchangeably referred to as PD-1, PD1, PDCD1, PDCD-1, SLEB2, SLE1 and CD279.

In humans, PD-1 typically has the sequence as disclosed in UniProtKB Ref. Q15116, incorporated herein by reference.

Programmed death-ligand 1 is herein interchangeably referred to as PDL-1, PD-LI, PDL1, PDCD1 L1, PDCD1LG1, CD274, B7-H1, B7-H, B7H1.

In humans, PD-L1 typically has the sequence as disclosed in UniProtKB Ref. Q9NZQ7, incorporated herein by reference.

In some embodiments, the anti-PD1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK 04, AK 05, BCD-100, BI-754091, CBT-501, CC-90006, GLS-010, HLX10, IBI-308, JNJ-3283, JS001, LZM009, MED10680 (AMP-514), REGN-2810, SHR-1210, Sym021, TSR-042, or XmAb20717.

In some embodiments, the PD-1 inhibitor is a bispecific antibody specific for PD-1 and VEGF. In some embodiments, the bispecific antibody is ivonescimab (SMT112). In some embodiments, the bispecific antibody is BNT327. In some embodiments, the bispecific antibody is SYN-2510.

In some embodiments, the anti-PDL1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-07Z.

An additional therapeutic agent may be an anti-TIGIT antibody, such as MBSA43, BMS-986207, MK-7684, COM902, AB154, MTIG7192A or OMP-313M32 (etigilimab).

In some embodiments, the combination therapy includes a RAS(ON) multi-selective inhibitor of the disclosure and an anti-CCR8 antibody. In an embodiment, the anti-CCR8 antibody is an afucosylated antibody. In an embodiment, the anti-CCR8 antibody is a depleting antibody. In an embodiment, the anti-CCR8 antibody has ADCC activity. In an embodiment, the anti-CCR8 antibody is a neutralizing antibody. In an embodiment, the anti-CCR8 antibody is not a neutralizing antibody. In an embodiment, the anti-CCR8 antibody is BMS-986340. In an embodiment, the anti-CCR8 antibody is GS-1811. In an embodiment, the anti-CCR8 antibody is ABBV-514. In an embodiment, the anti-CCR8 antibody is LM-108. In an embodiment, the anti-CCR8 antibody is S-531011. In an embodiment, the anti-CCR8 antibody is BAY3375968. In an embodiment, the anti-CCR8 antibody is SRFI 14. In an embodiment, the anti-CCR8 antibody is CM369. In an embodiment, the anti-CCR8 antibody is ZL-1218. In an embodiment, the anti-CCR8 antibody is IPG0521. In an embodiment, the anti-CCR8 antibody is an anti-CCR8 antibody disclosed in WO 2025076288, WO 2022256563, WO2022004760, WO2022136649, WO 2021142002, WO 2021194942, WO 2021260206, WO 2021260208, WO 2021260210, WO 2021260209, WO 2021152186, WO 2020138489, and WO 2018181425 which are incorporated herein by reference including the structures disclosed therein.

In some embodiments, the combination therapy includes a RAS(ON) multi-selective inhibitor of the disclosure and a cancer vaccine composition. In some embodiments, the cancer vaccine composition is ELI-002 2P, ELI-002 7P, HB-700, mRNA-4157, mRNA-5671, BNT111, GVAX Pancreas, IMA901, DCVax, SOT101, Sipuleucel-T, PROSTVAC-VF or TG01.

In some embodiments, the combination therapy includes a RAS(ON) multi-selective inhibitor of the disclosure and an additional therapy or therapeutic agent selected from group consisting of RAS pathway targeted therapeutic agents, kinase-targeted therapeutics, mTORC1 inhibitors or degraders, YAP inhibitors or degraders, proteasome inhibitors or degraders, HSP90 inhibitors or degraders, farnesyl transferase inhibitors or degraders, PTEN inhibitors or degraders, signal transduction pathway inhibitors or degraders, checkpoint inhibitors, modulators of the apoptosis pathway, chemotherapeutics, angiogenesis-targeted therapies, immune-targeted agents, radiotherapy, and combinations thereof.

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib): Casodex (bicalutarnide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammaII and calicheamicin omegaII (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Noivadex™); raioxifene; arornatase inhibiting 4(5)-imidazoles; 4-hydroxytarnoxifen; trioxifene; keoxifene; LY 117018; onapristone; torernifene (Fareston®); flutanide, nilutamide, bicalutamide, leuprolide, goserelin; chlorambucil: Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelaamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, PI3K/Akt inhibitors (e.g., perifosine), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS(ON) inhibitor of the present disclosure is used in combination with a MEK inhibitor and a SOS1 inhibitor. In some embodiments, a RAS(ON) inhibitor of the present disclosure is used in combination with a PD-L1 inhibitor and a SOS1 inhibitor. In some embodiments, a RAS(ON) inhibitor of the present disclosure is used in combination with a PD-L1 inhibitor and a SHP2 inhibitor. In some embodiments, a RAS(ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(i):186-192; Thompson et al., Clin. Cancer Res. 2007, 13(6):1757-1761; and WO 2006121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO 2010003118 and WO 2011090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO 2011028683, WO 2013039954, WO 2005007190, WO 2007133822, WO 2005055808, WO 199940196, WO 200103720, WO 199920758, WO 2006083289, WO 2005115451, and WO 2011051726.

Another example of a therapeutic agent that may be used in combination with a RAS(ON) multi-selective inhibitor of the present disclosure is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO 199633172, WO 199627583, WO 199807697, WO 199803516, WO 199834918, WO 199834915, WO 199833768, WO 199830566, WO 199005719, WO 199952910, WO 199952889, WO 199929667, WO 1999007675, EP 0606046, EP 0780386, EP 1786785, EP 1181017, EP 0818442, EP 1004578, and US 20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as Vectibix® (panitumumab), erlotinib (Tarceva®), anti-AngI and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US 20030162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 20020042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists (ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with a RAS(ON) multi-selective inhibitor of the present disclosure include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with a RAS(ON) multi-selective inhibitor of the present disclosure is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with a RAS(ON) multi-selective inhibitor of the present disclosure include ivonescimab, ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a RAS(ON) multi-selective inhibitor of the invention and any of the therapies described herein can be simultaneously administered, wherein both the agents are present in separate formulations.

In another alternative, a RAS(ON) multi-selective inhibitor of the present disclosure can be administered and followed by any of the therapies described herein, or vice versa. In some embodiments of the separate administration protocol, a RAS(ON) multi-selective inhibitor of the invention and any of the therapies described herein are administered a few minutes apart, or a few hours apart, or a few days apart.

In some embodiments of any of the methods described herein, the first therapy (e.g., a RAS(ON) multi-selective inhibitor of the disclosure) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

### Embodiments

Embodiment 1: A method of treating a RAS protein-related disease in a subject in need thereof, the method comprising administering to the subject a RAS(ON) multi-selective inhibitor on an intermittent dosing regimen.

Embodiment 2: The method of embodiment 1, wherein the subject has a mutation of RAS.

Embodiment 3: The method of embodiment 2, wherein the mutation of RAS is a KRAS mutation.

Embodiment 4: The method of embodiment 2 or 3, wherein the mutation is at G12X, wherein X is A, C, D, V, S or R amino acid residue.

Embodiment 5: The method of embodiment 2 or 3, wherein the mutation is at G13X, wherein X is A, C, D, V, S or R amino acid residue.

Embodiment 6: The method of embodiment 2 or 3, wherein the mutation is at Q61X, wherein X is A, C, D, V, S, R, H, K, or L amino acid residue.

Embodiment 7: The method of any one of embodiments 1-6, wherein the RAS protein-related disease is cancer.

Embodiment 8: The method of embodiment 7, wherein the cancer is lung cancer, pancreatic cancer, or colorectal cancer.

Embodiment 9: The method of embodiment 8, wherein the lung cancer is non-small cell lung cancer.

Embodiment 10: The method of embodiment 8, where the pancreatic cancer is pancreatic ductal adenocarcinoma.

Embodiment 11: The method of any one of embodiments 1-10, wherein the RAS(ON) multi-selective inhibitor has a K_{D}1 of less than 500 nM.

Embodiment 12: The method of any one of embodiments 1-10, wherein the RAS(ON) multi-selective inhibitor has a K_{D}1 of less than 100 nM.

Embodiment 13: The method of any one of embodiments 1-10, wherein the RAS(ON) multi-selective inhibitor has a K_{D}1 of less than 50 nM.

Embodiment 14: The method of any one of embodiments 1-10, wherein the RAS(ON) multi-selective inhibitor has a K_{D}1 of less than 10 nM.

Embodiment 15: The method of any one of embodiments 1-10, wherein the RAS(ON) multi-selective inhibitor has a K_{D}1 of 0.1 nM to 500 nM.

Embodiment 16: The method of any one of embodiments 11-15, wherein the K_{D}1 value of a RAS(ON) multi-selective inhibitor binding to cyclophilin A (CypA) is determined using surface plasmon resonance (SPR), Fluorescence Polarization (FP) or isothermal titration calorimetry (ITC).

Embodiment 17: The method of embodiment 16, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 18: The method of any one of embodiments 1-17, wherein the RAS(ON) multi-selective inhibitor has a blood or plasma half-life of 24 hours or longer.

Embodiment 19: The method of any one of embodiments 1-17, wherein the RAS(ON) multi-selective inhibitor has a blood or plasma half-life of 12 hours or longer.

Embodiment 20: The method of any one of embodiments 1-17, wherein the RAS(ON) multi-selective inhibitor has a blood or plasma half-life of 6 hours or longer.

Embodiment 21: The method of any one of embodiments 1-20, wherein the RAS(ON) multi-selective inhibitor has a tissue half-life of 48 hours or longer.

Embodiment 22: The method of any one of embodiments 1-20, wherein the RAS(ON) multi-selective inhibitor has a tissue half-life of 24 hours or longer.

Embodiment 23: The method of any one of embodiments 1-20, wherein the RAS(ON) multi-selective inhibitor has a tissue half-life of 12 hours or longer.

Embodiment 24: The method of any one of embodiments 1-20, wherein the RAS(ON) multi-selective inhibitor has a tissue half-life of 6 hours or longer.

Embodiment 25: The method of any one of embodiments 21-24, wherein the tissue is tumor tissue or normal tissue.

Embodiment 26: The method of any one of embodiments 18-25, wherein the blood or tissue half-life of the RAS(ON) multi-selective inhibitor is determined using noncompartmental analysis in pharmacokinetic software Phoenix WinNonlin.

Embodiment 27: The method of any one of embodiments 18-26, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 28: The method of any one of embodiments 1-27, wherein the RAS(ON) multi-selective inhibitor has a clearance rate of 0.4 L/h/kg or slower.

Embodiment 29: The method of any one of embodiments 1-27, wherein the RAS(ON) multi-selective inhibitor has a clearance rate of 0.35 L/h/kg or slower.

Embodiment 30: The method of any one of embodiments 1-27, wherein the RAS(ON) multi-selective inhibitor has a clearance rate of 0.25 L/h/kg or slower.

Embodiment 31: The method of any one of embodiments 1-27, wherein the RAS(ON) multi-selective inhibitor has a clearance rate of 0.1 L/h/kg or slower.

Embodiment 32: The method of any one of embodiments 1-27, wherein the RAS(ON) multi-selective inhibitor has a clearance rate of 0.01 L/h/kg or slower.

Embodiment 33: The method of any one of embodiments 28-32, wherein the clearance rate is determined in a preclinical species or in a human subject.

Embodiment 34: The method of any one of embodiments 28-33, wherein the clearance rate is from noncompartmental analysis using measured inhibitor concentrations over time in one or more blood samples.

Embodiment 35: The method of any one of embodiments 28-33, wherein the clearance rate is determined in blood from preclinical species and allometrically scaled to human.

Embodiment 36: The method of any one of embodiments 28-35, wherein the clearance rate is determined by applying PK simulation and modeling approaches to estimate clearance based on in silico human physiological conditions.

Embodiment 37: The method of any one of embodiments 1-36, wherein the intermittent dosing regimen comprises one dosing day followed by at least one day without dosing.

Embodiment 38: The method of any one of embodiments 1-36, wherein the intermittent dosing regimen comprises at least two consecutive dosing days followed by at least one day without dosing.

Embodiment 39: The method of any one of embodiments 1-36, wherein the intermittent dosing regimen comprises at least three consecutive dosing days followed by at least one day without dosing.

Embodiment 40: The method of any one of embodiments 1-36, wherein the intermittent dosing regimen comprises at least four consecutive dosing days followed by at least one day without dosing.

Embodiment 41: The method of any one of embodiments 1-36, wherein the intermittent dosing regimen comprises at least five consecutive dosing days followed by at least one day without dosing.

Embodiment 42: The method of any one of embodiments 1-36, wherein the intermittent dosing regimen comprises at least six consecutive dosing days followed by at least one day without dosing.

Embodiment 43: The method of any one of embodiments 1-36, wherein each dosing regimen comprises five dosing days and two days without dosing.

Embodiment 44: The method of any one of embodiments 1-36, wherein each dosing regimen comprises four dosing days and three days without dosing.

Embodiment 45: The method of any one of embodiments 1-36, wherein each dosing regimen comprises three dosing days and four days without dosing.

Embodiment 46: The method of any one of embodiments 1-36, wherein each dosing regimen comprises two dosing days and five days without dosing.

Embodiment 47: The method of any one of embodiments 1-36, wherein each dosing regimen comprises one dosing day and six days without dosing.

Embodiment 48: The method of any one of embodiments 1-36, wherein each dosing regimen comprises seven consecutive days of dosing (e.g., Q2W).

Embodiment 49: The method of any one of embodiments 1-36, wherein the RAS(ON) multi-selective inhibitor is administered Q2D.

Embodiment 50: The method of any one of embodiments 1-49, wherein the intermittent dosing regimen is repeated.

Embodiment 51: The method of any one of embodiments 1-50, wherein the daily dose of the RAS(ON) multi-selective inhibitor administered is between 10 mg to 2000 mg.

Embodiment 52: The method of embodiment 51, wherein the daily dose of the RAS(ON) multi-selective inhibitor is administered once per day.

Embodiment 53: The method of embodiment 51, wherein the daily dose of the RAS(ON) multi-selective inhibitor is divided equally into twice per day.

Embodiment 54: The method of any one of embodiments 1-53, wherein the method further comprises administering an additional therapeutic agent.

Embodiment 55: The method of embodiment 50, wherein the additional therapeutic agent is a second RAS inhibitor, a SOS1 inhibitor, a SHP inhibitor, a MEK inhibitor, a RAF inhibitor, a ERK inhibitor, a MAPK inhibitor, a PKA inhibitor, a FAK inhibitor, a ROCK inhibitor, a MSK1 inhibitor, a RSK inhibitor, an ALK inhibitor, an EGFR inhibitor, a HER2 inhibitor, a MET inhibitor, an AXL inhibitor, an IGFR inhibitor, a RET inhibitor, a ROS1 inhibitor, PDGFR inhibitor, an FGF inhibitor, a VEGF inhibitor, a PI3K inhibitor, an AKT inhibitor, an mTOR inhibitor, an MNK inhibitor, an eIF4 inhibitor, a Wee1 inhibitor, a CHK inhibitor, an ATM inhibitor, an ATR inhibitor, a PARP inhibitor, a DNA-PK inhibitor, a CDK inhibitor, an Aurora kinase inhibitor, a PLK inhibitor, a DYRK1 inhibitor, an ULK1 inhibitor, a VPS inhibitor, a micropinocytosis inhibitor, a beta-catenin inhibitor, a PORCN inhibitor, a GSK3 inhibitor, a CLK inhibitor, a JAK inhibitor, a STAT inhibitor, a HDAC inhibitor, a BET inhibitor, an EZH2 inhibitor, a Co-REST inhibitor, an EP300 inhibitor, an LSD1 inhibitor, a PRMT5 inhibitor, a MAT2A inhibitor, a DOT1L inhibitor, a UBA1 inhibitor, a ribonucleotide reductase inhibitor, a farnesyl transferase inhibitor, a casein kinase inhibitor, a FLT3 inhibitor, TGF-beta pathway inhibitor, a HSP90 inhibitor, a Glutathione Peroxidase 4 (GPX4) inhibitor, a NRF2 inhibitor, a TEA domain (TEAD) inhibitor, a NOTCH/Gamma secretase inhibitor, a Hedgehog inhibitor, a NFkappa-beta pathway inhibitor, surgery, radiation, chemotherapy, T cell adoptive transfer therapy, a Claudin-18 targeting agent, anti-CTLA, anti-PD-1, anti-PDL1, a B7-H3 inhibitor or antagonist, a B7-H4 inhibitor or antagonist, a BTLA inhibitor or antagonist, a HVEM inhibitor or antagonist, a TIM3 inhibitor or antagonist, a GAL9 inhibitor or antagonist, a LAG3 inhibitor or antagonist, a VISTA inhibitor or antagonist, a KIR inhibitor or antagonist, a 2B4 inhibitor or antagonist, a CD160 inhibitor or antagonist, a CGEN-15049 inhibitor or antagonist, an A2aR inhibitor or antagonist, an A2bR inhibitor or antagonist, an A2aR/A2bR inhibitor or antagonist, B-7 family ligands, and/or a RAS targeting cancer vaccine.

Embodiment 56: The method of embodiment 55, wherein the first RAS(ON) multi-selective inhibitor and the second RAS inhibitor are not identical.

Embodiment 57: The method of embodiment 56, wherein the second RAS inhibitor is a RAS(ON) mutant-selective inhibitor.

Embodiment 58: The method of embodiment 56, wherein the second RAS inhibitor is a RAS(OFF) inhibitor.

Embodiment 59: The method of embodiment 55-56 or 58, wherein the second RAS inhibitor is a pan-KRAS inhibitor.

Embodiment 60: The method of embodiment 59, wherein the pan-KRAS inhibitor is ERAS-4001.

Embodiment 61: The method of any one of embodiments 1-25, wherein the RAS(ON) multi-selective inhibitor is

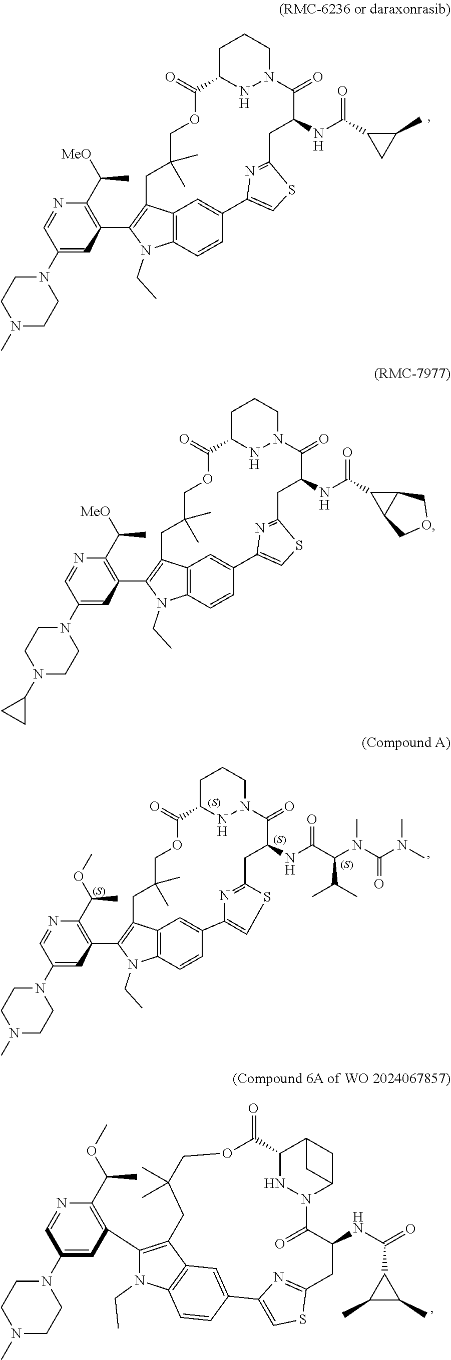

### or ERAS-0015.

Embodiment 62: The method of any one of embodiments 1-61, wherein the RAS(ON) multi-selective inhibitor exposure in normal tissues is decreased during non-dosing days while maintaining antitumor activity.

Embodiment 63: The method of any one of embodiments 1-62, wherein the subject does not exhibit any dose limiting toxicity.

Embodiment 64: The method of any one of embodiments 1-62, wherein the frequency or severity of treatment related adverse events are reduced compared to the frequency or severity of treatment related events of daily administration of the RAS(ON) multi-selective inhibitor.

Embodiment 65: The method of embodiment 64, wherein the treatment related adverse event is an on-target RAS-pathway mediated toxicity.

Embodiment 66: The method of embodiment 64 or 65, wherein the treatment related adverse event is rash or GI-related toxicity.

Embodiment 67: A method of treating a RAS protein-related disease, comprising administering to a subject in need thereof a RAS(ON) multi-selective inhibitor and an additional RAS inhibitor, wherein the RAS(ON) multi-selective inhibitor is administered on an intermittent regimen.

Embodiment 68: The method of embodiment 67, wherein the additional RAS inhibitor is administered on a daily dosing regimen (i.e., QD) or on an intermittent dosing regimen.

Embodiment 69: The method of embodiments 67 or 68, wherein the RAS(ON) multi-selective inhibitor and the additional RAS inhibitor are not identical.

Embodiment 70: The method of any one of embodiments 68-69, wherein the additional RAS inhibitor is a RAS(OFF) inhibitor.

Embodiment 71: The method of any one of embodiments 67-70, wherein the additional RAS inhibitor is a pan-KRAS inhibitor.

Embodiment 72: The method of any one of embodiments 67-71, wherein the pan-KRAS inhibitor is ERAS-4001.

Embodiment 73: The method of any one of embodiments 67-72, wherein the subject has a mutation of RAS.

Embodiment 74: The method of embodiment 73, wherein the mutation of RAS is a KRAS mutation.

Embodiment 75: The method of embodiment 73 or 74, wherein the mutation is at G12X, wherein X is A, C, D, V, S or R amino acid residue.

Embodiment 76: The method of embodiment 73 or 74, wherein the mutation is at G13X, wherein X is A, C, D, V, S or R amino acid residue.

Embodiment 77: The method of embodiment 73 or 74, wherein the mutation is at Q61X, wherein X is A, C, D, V, S, R, H, K, or L amino acid residue.

Embodiment 78: The method of any one of embodiments 67-77, wherein the RAS protein-related disease is cancer.

Embodiment 79: The method of embodiment 78, wherein the cancer is lung cancer, pancreatic cancer, or colorectal cancer.

Embodiment 80: The method of embodiment 79, wherein the lung cancer is non-small cell lung cancer.

Embodiment 81: The method of embodiment 79, where the pancreatic cancer is pancreatic ductal adenocarcinoma.

Embodiment 82: The method of any one of embodiments 67-81, wherein the RAS(ON) multi-selective inhibitor has a K_{D}1 of less than 500 nM.

Embodiment 83: The method of any one of embodiments 67-81, wherein the RAS(ON) multi-selective inhibitor has a K_{D}1 of less than 100 nM.

Embodiment 84: The method of any one of embodiments 67-81, wherein the RAS(ON) multi-selective inhibitor has a K_{D}1 of less than 50 nM.

Embodiment 85: The method of any one of embodiments 67-81, wherein the RAS(ON) multi-selective inhibitor has a K_{D}1 of less than 10 nM.

Embodiment 86: The method of any one of embodiments 67-81, wherein the RAS(ON) multi-selective inhibitor has a K_{D}1 of 0.1 nM to 500 nM.

Embodiment 87: The method of any one of embodiments 82-86, wherein the K_{D}1 value of a RAS(ON) multi-selective inhibitor binding to cyclophilin A (CypA) is determined using surface plasmon resonance (SPR), Fluorescence Polarization (FP), or isothermal titration calorimetry (ITC).

Embodiment 88: The method of embodiment 87, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 89: The method of any one of embodiments 67-88, wherein the RAS(ON) multi-selective inhibitor has a blood or plasma half-life of 24 hours or longer.

Embodiment 90: The method of any one of embodiments 67-88, wherein the RAS(ON) multi-selective inhibitor has a blood or plasma half-life of 12 hours or longer.

Embodiment 91: The method of any one of embodiments 67-88, wherein the RAS(ON) multi-selective inhibitor has a blood or plasma half-life of 6 hours or longer.

Embodiment 92: The method of any one of embodiments 67-91, wherein the RAS(ON) multi-selective inhibitor has a tissue half-life of 48 hours or longer.

Embodiment 93: The method of any one of embodiments 67-91, wherein the RAS(ON) multi-selective inhibitor has a tissue half-life of 24 hours or longer.

Embodiment 94: The method of any one of embodiments 67-91, wherein the RAS(ON) multi-selective inhibitor has a tissue half-life of 12 hours or longer.

Embodiment 95: The method of any one of embodiments 67-91, wherein the RAS(ON) multi-selective inhibitor has a tissue half-life of 6 hours or longer.

Embodiment 96: The method of any one of embodiments 92-95, wherein the tissue is tumor tissue or normal tissue.

Embodiment 97: The method of any one of embodiments 89-96, wherein the blood or tissue half-life of the RAS(ON) multi-selective inhibitor is determined using noncompartmental analysis in pharmacokinetic software Phoenix WinNonlin.

Embodiment 98: The method of any one of embodiments 89-97, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 99: The method of any one of embodiments 67-98, wherein the RAS(ON) multi-selective inhibitor has a clearance rate of 0.4 L/h/kg or slower.

Embodiment 100: The method of any one of embodiments 67-98, wherein the RAS(ON) multi-selective inhibitor has a clearance rate of 0.35 L/h/kg or slower.

Embodiment 101: The method of any one of embodiments 67-98, wherein the RAS(ON) multi-selective inhibitor has a clearance rate of 0.25 L/h/kg or slower.

Embodiment 102: The method of any one of embodiments 67-98, wherein the RAS(ON) multi-selective inhibitor has a clearance rate of 0.1 L/h/kg or slower.

Embodiment 103: The method of any one of embodiments 67-98, wherein the RAS(ON) multi-selective inhibitor has a clearance rate of 0.01 L/h/kg or slower.

Embodiment 104: The method of any one of embodiments 99-103, wherein the clearance rate is determined in a preclinical species or in a human subject.

Embodiment 105: The method of any one of embodiments 99-104, wherein the clearance rate is from noncompartmental analysis using measured inhibitor concentrations over time in one or more blood samples.

Embodiment 106: The method of any one of embodiments 99-105, wherein the clearance rate is determined in blood from preclinical species and allometrically scaled to human.

Embodiment 107: The method of any one of embodiments 99-106, wherein the clearance rate is determined by applying PK simulation and modeling approaches to estimate clearance based on in silico human physiological conditions.

Embodiment 108: The method of any one of embodiments 67-107, wherein the RAS(ON) multi-selective inhibitor is administered on the first, third, and fifth day of the intermittent dosing regimen and not administered on the second, fourth and sixth day of the intermittent dosing regimen.

Embodiment 109: The method of embodiment 108, wherein the additional RAS inhibitor is administered on the second, fourth and sixth day and not administered on the first, third, and fifth day of the intermittent dosing regimen.

Embodiment 110: The method of any one of embodiments 67-107, wherein the additional RAS inhibitor is administered on the first, third, and fifth day of the intermittent dosing regimen and not administered on the second, fourth and sixth day of the intermittent dosing regimen.

Embodiment 111: The method of embodiment 108, wherein the RAS(ON) multi-selective inhibitor is administered on the second, fourth and sixth day and not administered on the first, third, and fifth day of the intermittent dosing regimen.

Embodiment 112: The method of any one of embodiments 67-107, wherein the intermittent dosing regimen comprises one dosing day followed by at least one day without dosing.

Embodiment 113: The method of any one of embodiments 67-107, wherein the intermittent dosing regimen comprises at least two consecutive dosing days followed by at least one day without dosing.

Embodiment 114: The method of any one of embodiments 67-107, wherein the intermittent dosing regimen comprises at least three consecutive dosing days followed by at least one day without dosing.

Embodiment 115: The method of any one of embodiments 67-107, wherein the intermittent dosing regimen comprises at least four consecutive dosing days followed by at least one day without dosing.

Embodiment 116: The method of any one of embodiments 67-107, wherein the intermittent dosing regimen comprises at least five consecutive dosing days followed by at least one day without dosing.

Embodiment 117: The method of any one of embodiments 67-107, wherein the intermittent dosing regimen comprises at least six consecutive dosing days followed by at least one day without dosing.

Embodiment 118: The method of any one of embodiments 67-107, wherein each dosing regimen comprises five dosing days and two days without dosing.

Embodiment 119: The method of any one of embodiments 67-107, wherein each dosing regimen comprises four dosing days and three days without dosing.

Embodiment 120: The method of any one of embodiments 67-107, wherein each dosing regimen comprises three dosing days and four days without dosing.

Embodiment 121: The method of any one of embodiments 67-107, wherein each dosing regimen comprises two dosing days and five days without dosing.

Embodiment 122: The method of any one of embodiments 67-107, wherein each dosing regimen comprises one dosing day and six days without dosing.

Embodiment 123: The method of any one of embodiments 67-107, wherein each dosing regimen comprises seven consecutive days of dosing.

Embodiment 124: The method of any one of embodiments 67-107, wherein the RAS(ON) multi-selective inhibitor is administered Q2D.

Embodiment 125: The method of any one of embodiments 67-124, wherein the intermittent dosing regimen is repeated.

Embodiment 126: The method of any one of embodiments 67-125, wherein the daily dose of the RAS(ON) multi-selective inhibitor administered is between 10 mg to 2000 mg.

Embodiment 127: The method of embodiment 126, wherein the daily dose of the RAS(ON) multi-selective inhibitor is administered once per day.

Embodiment 128: The method of embodiment 126, wherein the daily dose of the RAS(ON) multi-selective inhibitor is divided equally into twice per day.

Embodiment 129: The method of any one of embodiments 67-128, wherein the RAS(ON) multi-selective inhibitor is RMC-6236 (daraxonrasib), RMC-7977, Compound A, Compound 6A of WO 2024067857, or ERAS-0015.

Embodiment 130: The method of any one of embodiments 67-129, wherein the daily dose of the pan-KRAS inhibitor is between about 10 mg to about 1000 mg.

Embodiment 131: The method of any one of embodiments 67-130, wherein the RAS(ON) multi-selective inhibitor exposure in normal tissues is decreased during non-dosing days while maintaining antitumor activity.

Embodiment 132: The method of any one of embodiments 67-131, wherein the subject does not exhibit any dose limiting toxicity.

Embodiment 133: The method of any one of embodiments 67-131, wherein the frequency or severity of treatment related adverse events are reduced compared to the frequency or severity of treatment related events of daily administration of the RAS(ON) multi-selective inhibitor.

Embodiment 134: The method of embodiment 133, wherein the treatment related adverse event is an on-target RAS-pathway mediated toxicity.

Embodiment 135: The method of embodiment 133 or 134, wherein the treatment related adverse event is rash or GI-related toxicity.

Embodiment 136: A method of reducing RAS inhibition in normal tissues of a subject in need of and receiving a RAS(ON) multi-selective inhibitor, the method comprising administering the RAS(ON) multi-selective inhibitor on an intermittent dosing regimen.

Embodiment 137: The method of embodiment 136, wherein the subject has a mutation of RAS.

Embodiment 138: The method of embodiment 137, wherein the mutation of RAS is a KRAS mutation.

Embodiment 139: The method of embodiment 137 or 138, wherein the mutation is at G12X, wherein X is A, C, D, V, S or R amino acid residue.

Embodiment 140: The method of embodiment 137 or 138, wherein the mutation is at G13X, wherein X is A, C, D, V, S or R amino acid residue.

Embodiment 141: The method of embodiment 137 or 138, wherein the mutation is at Q61X, wherein X is A, C, D, V, S, R, H, K, or L amino acid residue.

Embodiment 142: The method of any one of embodiments 136-141, wherein the subject has a RAS protein-related disease, wherein the RAS-protein related disease is cancer.

Embodiment 143: The method of embodiment 142, wherein the cancer is lung cancer, pancreatic cancer, or colorectal cancer.

Embodiment 144: The method of embodiment 143, wherein the lung cancer is non-small cell lung cancer.

Embodiment 145: The method of embodiment 144, where the pancreatic cancer is pancreatic ductal adenocarcinoma.

Embodiment 146: The method of any one of embodiments 136-145, wherein the RAS(ON) multi-selective inhibitor has a K_{D}1 of less than 500 nM.

Embodiment 147: The method of any one of embodiments 136-145, wherein the RAS(ON) multi-selective inhibitor has a K_{D}1 of less than 100 nM.

Embodiment 148: The method of any one of embodiments 136-145, wherein the RAS(ON) multi-selective inhibitor has a K_{D}1 of less than 50 nM.

Embodiment 149: The method of any one of embodiments 136-145, wherein the RAS(ON) multi-selective inhibitor has a K_{D}1 of less than 10 nM.

Embodiment 150: The method of any one of embodiments 136-145, wherein the RAS(ON) multi-selective inhibitor has a K_{D}1 of 0.1 nM to 500 nM.

Embodiment 151: The method of any one of embodiments 146-150, wherein the K_{D}1 value of a RAS(ON) multi-selective inhibitor binding to cyclophilin A (CypA) is determined using surface plasmon resonance (SPR), Fluorescence Polarization (FP) or isothermal titration calorimetry (ITC).

Embodiment 152: The method of embodiment 151, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 153: The method of any one of embodiments 136-152, wherein the RAS(ON) multi-selective inhibitor has a blood or plasma half-life of 24 hours or longer.

Embodiment 154: The method of any one of embodiments 136-152, wherein the RAS(ON) multi-selective inhibitor has a blood or plasma half-life of 12 hours or longer.

Embodiment 155: The method of any one of embodiments 136-152, wherein the RAS(ON) multi-selective inhibitor has a blood or plasma half-life of 6 hours or longer.

Embodiment 156: The method of any one of embodiments 136-155, wherein the RAS(ON) multi-selective inhibitor has a tissue half-life of 48 hours or longer.

Embodiment 157: The method of any one of embodiments 136-155, wherein the RAS(ON) multi-selective inhibitor has a tissue half-life of 24 hours or longer.

Embodiment 158: The method of any one of embodiments 136-155, wherein the RAS(ON) multi-selective inhibitor has a tissue half-life of 12 hours or longer.

Embodiment 159: The method of any one of embodiments 136-155, wherein the RAS(ON) multi-selective inhibitor has a tissue half-life of 6 hours or longer.

Embodiment 160: The method of any one of embodiments 156-159, wherein the tissue is tumor tissue or normal tissue.

Embodiment 161: The method of any one of embodiments 153-160, wherein the blood or tissue half-life of the RAS(ON) multi-selective inhibitor is determined using noncompartmental analysis in pharmacokinetic software Phoenix WinNonlin.

Embodiment 162: The method of any one of embodiments 153-161, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 163: The method of any one of embodiments 136-162, wherein the RAS(ON) multi-selective inhibitor has a clearance rate of 0.4 L/h/kg or slower.

Embodiment 164: The method of any one of embodiments 136-162, wherein the RAS(ON) multi-selective inhibitor has a clearance rate of 0.35 L/h/kg or slower.

Embodiment 165: The method of any one of embodiments 136-162, wherein the RAS(ON) multi-selective inhibitor has a clearance rate of 0.25 L/h/kg or slower.

Embodiment 166: The method of any one of embodiments 136-162, wherein the RAS(ON) multi-selective inhibitor has a clearance rate of 0.1 L/h/kg or slower.

Embodiment 167: The method of any one of embodiments 136-162, wherein the RAS(ON) multi-selective inhibitor has a clearance rate of 0.01 L/h/kg or slower.

Embodiment 168: The method of any one of embodiments 163-167, wherein the clearance rate is determined in a preclinical species or in a human subject.

Embodiment 169: The method of any one of embodiments 163-167, wherein the clearance rate is from noncompartmental analysis using measured inhibitor concentrations over time in one or more blood samples.

Embodiment 170: The method of any one of embodiments 163-167, wherein the clearance rate is determined in blood from preclinical species and allometrically scaled to human.

Embodiment 171: The method of any one of embodiments 163-170, wherein the clearance rate is determined by applying PK simulation and modeling approaches to estimate clearance based on in silico human physiological conditions.

Embodiment 172: The method of any one of embodiments 136-171, wherein the intermittent dosing regimen comprises one dosing day followed by at least one day without dosing.

Embodiment 173: The method of any one of embodiments 136-171, wherein the intermittent dosing regimen comprises at least two consecutive dosing days followed by at least one day without dosing.

Embodiment 174: The method of any one of embodiments 136-171, wherein the intermittent dosing regimen comprises at least three consecutive dosing days followed by at least one day without dosing.

Embodiment 175: The method of any one of embodiments 136-171, wherein the intermittent dosing regimen comprises at least four consecutive dosing days followed by at least one day without dosing.

Embodiment 176: The method of any one of embodiments 136-171, wherein the intermittent dosing regimen comprises at least five consecutive dosing days followed by at least one day without dosing.

Embodiment 177: The method of any one of embodiments 136-171, wherein the intermittent dosing regimen comprises at least six consecutive dosing days followed by at least one day without dosing.

Embodiment 178: The method of any one of embodiments 136-171, wherein each dosing regimen comprises five dosing days and two days without dosing.

Embodiment 179: The method of any one of embodiments 136-171, wherein each dosing regimen comprises four dosing days and three days without dosing.

Embodiment 180: The method of any one of embodiments 136-171, wherein each dosing regimen comprises three dosing days and four days without dosing.

Embodiment 181: The method of any one of embodiments 136-171, wherein each dosing regimen comprises two dosing days and five days without dosing.

Embodiment 182: The method of any one of embodiments 136-171, wherein each dosing regimen comprises one dosing day and six days without dosing.

Embodiment 183: The method of any one of embodiments 136-171, wherein each dosing regimen comprises seven consecutive days of dosing (e.g., Q2W).

Embodiment 184: The method of any one of embodiments 136-171, wherein the RAS(ON) multi-selective inhibitor is administered Q2D.

Embodiment 185: The method of any one of embodiments 136-184, wherein the intermittent dosing regimen is repeated.

Embodiment 186: The method of any one of embodiments 136-185, wherein the daily dose of the RAS(ON) multi-selective inhibitor administered is between 10 mg to 2000 mg.

Embodiment 187: The method of embodiment 186, wherein the daily dose of the RAS(ON) multi-selective inhibitor is administered once per day.

Embodiment 188: The method of embodiment 187, wherein the daily dose of the RAS(ON) multi-selective inhibitor is divided equally into twice per day.

Embodiment 189: The method of any one of embodiments 136-188, wherein the RAS(ON) multi-selective inhibitor is RMC-6236 (daraxonrasib), RMC-7977, Compound A, Compound 6A of WO 2024067857, or ERAS-0015.

Embodiment 190: The method of any one of embodiments 136-189, wherein the RAS(ON) multi-selective inhibitor exposure in normal tissues is decreased during non-dosing days while maintaining antitumor activity.

Embodiment 191: The method of any one of embodiments 136-190, wherein the subject does not exhibit a dose limiting toxicity.

Embodiment 192: The method of any one of embodiments 136-191, wherein the frequency or severity of treatment related adverse events are reduced compared to the frequency or severity of treatment related events of daily administration of the RAS(ON) multi-selective inhibitor.

Embodiment 193: The method of embodiment 192, wherein the treatment related adverse event is an on-target RAS-pathway mediated toxicity.

Embodiment 194: The method of embodiment 192 or 193, wherein the treatment related adverse event is rash or a GI-related toxicity.

Embodiment 195: A method of allowing reactivation of the RAS signaling in normal tissues of a subject in need of and receiving a RAS(ON) multi-selective inhibitor, the method comprising administering the RAS(ON) multi-selective inhibitor on an intermittent dosing regimen.

Embodiment 196: The method of embodiment 195, wherein the subject has a mutation of RAS.

Embodiment 197: The method of embodiment 196, wherein the mutation of RAS is a KRAS mutation.

Embodiment 198: The method of embodiment 196 or 197, wherein the mutation is at G12X, wherein X is A, C, D, V, S or R amino acid residue.

Embodiment 200: The method of embodiment 196 or 197, wherein the mutation is at G13X, wherein X is A, C, D, V, S or R amino acid residue.

Embodiment 201: The method of embodiment 196 or 197, wherein the mutation is at Q61X, wherein X is A, C, D, V, S, R, H, K, or L amino acid residue.

Embodiment 202: The method of any one of embodiments 195-201, wherein the subject has a RAS protein-related disease, wherein the RAS-protein related disease is cancer.

Embodiment 203: The method of embodiment 202, wherein the cancer is lung cancer, pancreatic cancer, or colorectal cancer.

Embodiment 204: The method of embodiment 203, wherein the lung cancer is non-small cell lung cancer.

Embodiment 205: The method of embodiment 204, where the pancreatic cancer is pancreatic ductal adenocarcinoma.

Embodiment 206: The method of any one of embodiments 195-205, wherein the RAS(ON) multi-selective inhibitor has a K_{D}1 of less than 500 nM.

Embodiment 207: The method of any one of embodiments 195-205, wherein the RAS(ON) multi-selective inhibitor has a K_{D}1 of less than 100 nM.

Embodiment 208: The method of any one of embodiments 195-205, wherein the RAS(ON) multi-selective inhibitor has a K_{D}1 of less than 50 nM.

Embodiment 209: The method of any one of embodiments 195-205, wherein the RAS(ON) multi-selective inhibitor has a K_{D}1 of less than 10 nM.

Embodiment 210: The method of any one of embodiments 195-205, wherein the RAS(ON) multi-selective inhibitor has a K_{D}1 of 0.1 nM to 500 nM.

Embodiment 211: The method of any one of embodiments 206-210, wherein the K_{D}1 value of a RAS(ON) multi-selective inhibitor binding to cyclophilin A (CypA) is determined using surface plasmon resonance (SPR), Fluorescence Polarization (FP) or isothermal titration calorimetry (ITC).

Embodiment 212: The method of embodiment 211, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 213: The method of any one of embodiments 195-212, wherein the RAS(ON) multi-selective inhibitor has a blood or plasma half-life of 24 hours or longer.

Embodiment 214: The method of any one of embodiments 195-212, wherein the RAS(ON) multi-selective inhibitor has a blood or plasma half-life of 12 hours or longer.

Embodiment 215: The method of any one of embodiments 195-212, wherein the RAS(ON) multi-selective inhibitor has a blood or plasma half-life of 6 hours or longer.

Embodiment 216: The method of any one of embodiments 195-215, wherein the RAS(ON) multi-selective inhibitor has a tissue half-life of 48 hours or longer.

Embodiment 217: The method of any one of embodiments 195-215, wherein the RAS(ON) multi-selective inhibitor has a tissue half-life of 24 hours or longer.

Embodiment 218: The method of any one of embodiments 195-215, wherein the RAS(ON) multi-selective inhibitor has a tissue half-life of 12 hours or longer.

Embodiment 219: The method of any one of embodiments 195-215, wherein the RAS(ON) multi-selective inhibitor has a tissue half-life of 6 hours or longer.

Embodiment 220: The method of any one of embodiments 216-219, wherein the tissue is tumor tissue or normal tissue.

Embodiment 221: The method of any one of embodiments 213-220, wherein the blood or tissue half-life of the RAS(ON) multi-selective inhibitor is determined using noncompartmental analysis in pharmacokinetic software Phoenix WinNonlin.

Embodiment 222: The method of any one of embodiments 213-221, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 223: The method of any one of embodiments 195-222, wherein the RAS(ON) multi-selective inhibitor has a clearance rate of 0.4 L/h/kg or slower.

Embodiment 224: The method of any one of embodiments 195-222, wherein the RAS(ON) multi-selective inhibitor has a clearance rate of 0.35 L/h/kg or slower.

Embodiment 225: The method of any one of embodiments 195-222, wherein the RAS(ON) multi-selective inhibitor has a clearance rate of 0.25 L/h/kg or slower.

Embodiment 226: The method of any one of embodiments 195-222, wherein the RAS(ON) multi-selective inhibitor has a clearance rate of 0.1 L/h/kg or slower.

Embodiment 227: The method of any one of embodiments 195-222, wherein the RAS(ON) multi-selective inhibitor has a clearance rate of 0.01 L/h/kg or slower.

Embodiment 228: The method of any one of embodiments 223-227, wherein the clearance rate is determined in a preclinical species or in a human subject.

Embodiment 229: The method of any one of embodiments 223-227, wherein the clearance rate is from noncompartmental analysis using measured inhibitor concentrations over time in one or more blood samples.

Embodiment 230: The method of any one of embodiments 223-227, wherein the clearance rate is determined in blood from preclinical species and allometrically scaled to human.

Embodiment 231: The method of any one of embodiments 223-230, wherein the clearance rate is determined by applying PK simulation and modeling approaches to estimate clearance based on in silico human physiological conditions.

Embodiment 232: The method of any one of embodiments 195-231, wherein the intermittent dosing regimen comprises one dosing day followed by at least one day without dosing.

Embodiment 233: The method of any one of embodiments 195-231, wherein the intermittent dosing regimen comprises at least two consecutive dosing days followed by at least one day without dosing.

Embodiment 234: The method of any one of embodiments 195-231, wherein the intermittent dosing regimen comprises at least three consecutive dosing days followed by at least one day without dosing.

Embodiment 235: The method of any one of embodiments 195-231, wherein the intermittent dosing regimen comprises at least four consecutive dosing days followed by at least one day without dosing.

Embodiment 236: The method of any one of embodiments 195-231, wherein the intermittent dosing regimen comprises at least five consecutive dosing days followed by at least one day without dosing.

Embodiment 237: The method of any one of embodiments 195-231, wherein the intermittent dosing regimen comprises at least six consecutive dosing days followed by at least one day without dosing.

Embodiment 238: The method of any one of embodiments 195-231, wherein each dosing regimen comprises five dosing days and two days without dosing.

Embodiment 239: The method of any one of embodiments 195-231, wherein each dosing regimen comprises four dosing days and three days without dosing.

Embodiment 240: The method of any one of embodiments 195-231, wherein each dosing regimen comprises three dosing days and four days without dosing.

Embodiment 241: The method of any one of embodiments 195-231, wherein each dosing regimen comprises two dosing days and five days without dosing.

Embodiment 242: The method of any one of embodiments 195-231, wherein each dosing regimen comprises one dosing day and six days without dosing.

Embodiment 243: The method of any one of embodiments 195-231, wherein each dosing regimen comprises seven consecutive days of dosing (e.g., Q2W).

Embodiment 244: The method of any one of embodiments 195-231, wherein the RAS(ON) multi-selective inhibitor is administered Q2D.

Embodiment 245: The method of any one of embodiments 195-184, wherein the intermittent dosing regimen is repeated.

Embodiment 246: The method of any one of embodiments 195-185, wherein the daily dose of the RAS(ON) multi-selective inhibitor administered is between 10 mg to 2000 mg.

Embodiment 247: The method of embodiment 246, wherein the daily dose of the RAS(ON) multi-selective inhibitor is administered once per day.

Embodiment 248: The method of embodiment 247, wherein the daily dose of the RAS(ON) multi-selective inhibitor is divided equally into twice per day.

Embodiment 249: The method of any one of embodiments 195-248, wherein the RAS(ON) multi-selective inhibitor is RMC-6236 (daraxonrasib), RMC-7977, Compound A, Compound 6A of WO 2024067857, or ERAS-0015.

Embodiment 250: The method of any one of embodiments 195-189, wherein the RAS(ON) multi-selective inhibitor exposure in normal tissues is decreased during non-dosing days while maintaining antitumor activity.

Embodiment 251: The method of any one of embodiments 1195-250, wherein the subject does not exhibit a dose limiting toxicity.

Embodiment 252: The method of any one of embodiments 195-251, wherein the frequency or severity of treatment related adverse events are reduced compared to the frequency or severity of treatment related events of daily administration of the RAS(ON) multi-selective inhibitor.

Embodiment 253: The method of embodiment 252, wherein the treatment related adverse event is an on-target RAS-pathway mediated toxicity.

Embodiment 254: The method of embodiment 252 or 253, wherein the treatment related adverse event is rash or a GI-related toxicity.

Embodiment 255: A method of reducing RAS(ON) multi-selective inhibitor retention in non-tumor tissues of a subject in need of and receiving a RAS(ON) multi-selective inhibitor, the method comprising administering the RAS(ON) multi-selective inhibitor on an intermittent dosing regimen thereby reducing RAS(ON) multi-selective inhibitor retention in non-tumor tissues relative to a daily dosing regimen.

Embodiment 256: The method of embodiment 255, wherein the subject has a mutation of RAS.

Embodiment 257: The method of embodiment 256, wherein the mutation of RAS is a KRAS mutation.

Embodiment 258: The method of embodiment 256 or 257, wherein the mutation is at G12X, wherein X is A, C, D, V, S or R amino acid residue.

Embodiment 259: The method of embodiment 256 or 257, wherein the mutation is at G13X, wherein X is A, C, D, V, S or R amino acid residue.

Embodiment 260: The method of embodiment 256 or 257, wherein the mutation is at Q61X, wherein X is A, C, D, V, S, R, H, K, or L amino acid residue.

Embodiment 261: The method of any one of embodiments 255-260, wherein the subject has a RAS protein-related disease, wherein the RAS-protein related disease is cancer.

Embodiment 262: The method of embodiment 261, wherein the cancer is lung cancer, pancreatic cancer, or colorectal cancer.

Embodiment 263: The method of embodiment 262, wherein the lung cancer is non-small cell lung cancer.

Embodiment 264: The method of embodiment 263, where the pancreatic cancer is pancreatic ductal adenocarcinoma.

Embodiment 265: The method of any one of embodiments 255-264, wherein the RAS(ON) multi-selective inhibitor has a K_{D}1 of less than 500 nM.

Embodiment 266: The method of any one of embodiments 255-264, wherein the RAS(ON) multi-selective inhibitor has a K_{D}1 of less than 100 nM.

Embodiment 267: The method of any one of embodiments 255-264, wherein the RAS(ON) multi-selective inhibitor has a K_{D}1 of less than 50 nM.

Embodiment 268: The method of any one of embodiments 255-264, wherein the RAS(ON) multi-selective inhibitor has a K_{D}1 of less than 10 nM.

Embodiment 269: The method of any one of embodiments 255-264, wherein the RAS(ON) multi-selective inhibitor has a K_{D}1 of 0.1 nM to 500 nM.

Embodiment 270: The method of any one of embodiments 265-269, wherein the K_{D}1 value of a RAS(ON) multi-selective inhibitor binding to cyclophilin A (CypA) is determined using surface plasmon resonance (SPR), Fluorescence Polarization (FP) or isothermal titration calorimetry (ITC).

Embodiment 271: The method of embodiment 270, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 272: The method of any one of embodiments 255-271, wherein the RAS(ON) multi-selective inhibitor has a blood or plasma half-life of 24 hours or longer.

Embodiment 273: The method of any one of embodiments 255-271, wherein the RAS(ON) multi-selective inhibitor has a blood or plasma half-life of 12 hours or longer.

Embodiment 274: The method of any one of embodiments 255-271, wherein the RAS(ON) multi-selective inhibitor has a blood or plasma half-life of 6 hours or longer.

Embodiment 275: The method of any one of embodiments 255-274, wherein the RAS(ON) multi-selective inhibitor has a tissue half-life of 48 hours or longer.

Embodiment 276: The method of any one of embodiments 255-274, wherein the RAS(ON) multi-selective inhibitor has a tissue half-life of 24 hours or longer.

Embodiment 277: The method of any one of embodiments 255-274, wherein the RAS(ON) multi-selective inhibitor has a tissue half-life of 12 hours or longer.

Embodiment 278: The method of any one of embodiments 255-274, wherein the RAS(ON) multi-selective inhibitor has a tissue half-life of 6 hours or longer.

Embodiment 279: The method of any one of embodiments 275-278, wherein the tissue is tumor tissue or normal tissue.

Embodiment 280: The method of any one of embodiments 272-279, wherein the blood or tissue half-life of the RAS(ON) multi-selective inhibitor is determined using noncompartmental analysis in pharmacokinetic software Phoenix WinNonlin.

Embodiment 281: The method of any one of embodiments 272-280, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 282: The method of any one of embodiments 255-281, wherein the RAS(ON) multi-selective inhibitor has a clearance rate of 0.4 L/h/kg or slower.

Embodiment 283: The method of any one of embodiments 255-281, wherein the RAS(ON) multi-selective inhibitor has a clearance rate of 0.35 L/h/kg or slower.

Embodiment 284: The method of any one of embodiments 255-281, wherein the RAS(ON) multi-selective inhibitor has a clearance rate of 0.25 L/h/kg or slower.

Embodiment 285: The method of any one of embodiments 255-281, wherein the RAS(ON) multi-selective inhibitor has a clearance rate of 0.1 L/h/kg or slower.

Embodiment 286: The method of any one of embodiments 255-281, wherein the RAS(ON) multi-selective inhibitor has a clearance rate of 0.01 L/h/kg or slower.

Embodiment 287: The method of any one of embodiments 282-286, wherein the clearance rate is determined in a preclinical species or in a human subject.

Embodiment 288: The method of any one of embodiments 282-286, wherein the clearance rate is from noncompartmental analysis using measured inhibitor concentrations over time in one or more blood samples.

Embodiment 289: The method of any one of embodiments 282-286, wherein the clearance rate is determined in blood from preclinical species and allometrically scaled to human.

Embodiment 290: The method of any one of embodiments 282-289, wherein the clearance rate is determined by applying PK simulation and modeling approaches to estimate clearance based on in silico human physiological conditions.

Embodiment 291: The method of any one of embodiments 255-290, wherein the intermittent dosing regimen comprises one dosing day followed by at least one day without dosing.

Embodiment 292: The method of any one of embodiments 255-290, wherein the intermittent dosing regimen comprises at least two consecutive dosing days followed by at least one day without dosing.

Embodiment 293: The method of any one of embodiments 255-290, wherein the intermittent dosing regimen comprises at least three consecutive dosing days followed by at least one day without dosing.

Embodiment 294: The method of any one of embodiments 255-290, wherein the intermittent dosing regimen comprises at least four consecutive dosing days followed by at least one day without dosing.

Embodiment 295: The method of any one of embodiments 255-290, wherein the intermittent dosing regimen comprises at least five consecutive dosing days followed by at least one day without dosing.

Embodiment 296: The method of any one of embodiments 255-290, wherein the intermittent dosing regimen comprises at least six consecutive dosing days followed by at least one day without dosing.

Embodiment 297: The method of any one of embodiments 255-290, wherein each dosing regimen comprises five dosing days and two days without dosing.

Embodiment 298: The method of any one of embodiments 255-290, wherein each dosing regimen comprises four dosing days and three days without dosing.

Embodiment 299: The method of any one of embodiments 255-290, wherein each dosing regimen comprises three dosing days and four days without dosing.

Embodiment 300: The method of any one of embodiments 255-290, wherein each dosing regimen comprises two dosing days and five days without dosing.

Embodiment 301: The method of any one of embodiments 255-290, wherein each dosing regimen comprises one dosing day and six days without dosing.

Embodiment 302: The method of any one of embodiments 255-290, wherein each dosing regimen comprises seven consecutive days of dosing (e.g., Q2W).

Embodiment 303: The method of any one of embodiments 255-290, wherein the RAS(ON) multi-selective inhibitor is administered Q2D.

Embodiment 304: The method of any one of embodiments 255-290, wherein the intermittent dosing regimen is repeated.

Embodiment 305: The method of any one of embodiments 255-290, wherein the daily dose of the RAS(ON) multi-selective inhibitor administered is between 10 mg to 2000 mg.

Embodiment 306: The method of embodiment 305, wherein the daily dose of the RAS(ON) multi-selective inhibitor is administered once per day.

Embodiment 307: The method of embodiment 306, wherein the daily dose of the RAS(ON) multi-selective inhibitor is divided equally into twice per day.

Embodiment 308: The method of any one of embodiments 255-307, wherein the RAS(ON) multi-selective inhibitor is RMC-6236 (daraxonrasib), RMC-7977, Compound A, Compound 6A of WO 2024067857, or ERAS-0015.

Embodiment 309: The method of any one of embodiments 255-308, wherein the RAS(ON) multi-selective inhibitor exposure in normal tissues is decreased during non-dosing days while maintaining antitumor activity.

Embodiment 310: The method of any one of embodiments 255-309, wherein the subject does not exhibit a dose limiting toxicity.

Embodiment 311: The method of any one of embodiments 255-310, wherein the frequency or severity of treatment related adverse events are reduced compared to the frequency or severity of treatment related events of daily administration of the RAS(ON) multi-selective inhibitor.

Embodiment 312: The method of embodiment 311, wherein the treatment related adverse event is an on-target RAS-pathway mediated toxicity.

Embodiment 313: The method of embodiment 311 or 312, wherein the treatment related adverse event is rash or a GI-related toxicity.

Embodiment 314: A method of reducing dose-limiting toxicities (DLT) associated with a RAS(ON) multi-selective inhibitor in a subject in need of and receiving a RAS(ON) multi-selective inhibitor, the method comprising administering the RAS(ON) multi-selective inhibitor on an intermittent dosing regimen, wherein DLTs are reduced relative to the RAS(ON) multi-selective inhibitor administered on a daily dosing regimen.

Embodiment 315: The method of embodiment 314, wherein the subject has a mutation of RAS.

Embodiment 316: The method of embodiment 315, wherein the mutation of RAS is a KRAS mutation.

Embodiment 317: The method of embodiment 315 or 316, wherein the mutation is at G12X, wherein X is A, C, D, V, S or R amino acid residue.

Embodiment 318: The method of embodiment 315 or 316, wherein the mutation is at G13X, wherein X is A, C, D, V, S or R amino acid residue.

Embodiment 319: The method of embodiment 315 or 316, wherein the mutation is at Q61X, wherein X is A, C, D, V, S, R, H, K, or L amino acid residue.

Embodiment 320: The method of any one of embodiments 314-319, wherein the subject has a RAS protein-related disease, wherein the RAS-protein related disease is cancer.

Embodiment 321: The method of embodiment 320, wherein the cancer is lung cancer, pancreatic cancer, or colorectal cancer.

Embodiment 322: The method of embodiment 321, wherein the lung cancer is non-small cell lung cancer.

Embodiment 323: The method of embodiment 322, where the pancreatic cancer is pancreatic ductal adenocarcinoma.

Embodiment 324: The method of any one of embodiments 314-323, wherein the RAS(ON) multi-selective inhibitor has a K_{D}1 of less than 500 nM.

Embodiment 325: The method of any one of embodiments 314-323, wherein the RAS(ON) multi-selective inhibitor has a K_{D}1 of less than 100 nM.

Embodiment 326: The method of any one of embodiments 314-323, wherein the RAS(ON) multi-selective inhibitor has a K_{D}1 of less than 50 nM.

Embodiment 327: The method of any one of embodiments 314-323, wherein the RAS(ON) multi-selective inhibitor has a K_{D}1 of less than 10 nM.

Embodiment 328: The method of any one of embodiments 314-323, wherein the RAS(ON) multi-selective inhibitor has a K_{D}1 of 0.1 nM to 500 nM.

Embodiment 329: The method of any one of embodiments 324-328, wherein the K_{D}1 value of a RAS(ON) multi-selective inhibitor binding to cyclophilin A (CypA) is determined using surface plasmon resonance (SPR), Fluorescence Polarization (FP), or isothermal titration calorimetry (ITC).

Embodiment 330: The method of embodiment 329, wherein the K_{D}1 is assessed by SPR using a Biacore 8K instrument, immobilizing CypA on a sensor chip, flowing varying RAS(ON) multi-selective inhibitor concentrations over the chip in assay buffer and fitting the SPR sensorgrams using either a steady state affinity model or a 1:1 binding (kinetic) model.

Embodiment 331: The method of any one of embodiments 314-330, wherein the RAS(ON) multi-selective inhibitor has a blood or plasma half-life of 24 hours or longer.

Embodiment 332: The method of any one of embodiments 314-330, wherein the RAS(ON) multi-selective inhibitor has a blood or plasma half-life of 12 hours or longer.

Embodiment 333: The method of any one of embodiments 314-330, wherein the RAS(ON) multi-selective inhibitor has a blood or plasma half-life of 6 hours or longer.

Embodiment 334: The method of any one of embodiments 314-333, wherein the RAS(ON) multi-selective inhibitor has a tissue half-life of 48 hours or longer.

Embodiment 335: The method of any one of embodiments 314-333, wherein the RAS(ON) multi-selective inhibitor has a tissue half-life of 24 hours or longer.

Embodiment 336: The method of any one of embodiments 314-333, wherein the RAS(ON) multi-selective inhibitor has a tissue half-life of 12 hours or longer.

Embodiment 337: The method of any one of embodiments 314-333, wherein the RAS(ON) multi-selective inhibitor has a tissue half-life of 6 hours or longer.

Embodiment 338: The method of any one of embodiments 334-337, wherein the tissue is tumor tissue or normal tissue.

Embodiment 339: The method of any one of embodiments 331-338, wherein the blood or tissue half-life of the RAS(ON) multi-selective inhibitor is determined using noncompartmental analysis in pharmacokinetic software Phoenix WinNonlin.

Embodiment 340: The method of any one of embodiments 331-339, wherein the blood or tissue half-life is determined by measuring the concentration of the RAS(ON) multi-selective inhibitor in blood or tissue samples obtained over time by high-performance liquid chromatography (HPLC), mass spectrometry (MS), LC-MS/MS or enzyme-linked immunosorbent assay (ELISA).

Embodiment 341: The method of any one of embodiments 314-340, wherein the RAS(ON) multi-selective inhibitor has a clearance rate of 0.4 L/h/kg or slower.

Embodiment 342: The method of any one of embodiments 314-340, wherein the RAS(ON) multi-selective inhibitor has a clearance rate of 0.35 L/h/kg or slower.

Embodiment 343: The method of any one of embodiments 314-340, wherein the RAS(ON) multi-selective inhibitor has a clearance rate of 0.25 L/h/kg or slower.

Embodiment 344: The method of any one of embodiments 314-340, wherein the RAS(ON) multi-selective inhibitor has a clearance rate of 0.1 L/h/kg or slower.

Embodiment 345: The method of any one of embodiments 314-340, wherein the RAS(ON) multi-selective inhibitor has a clearance rate of 0.01 L/h/kg or slower.

Embodiment 346: The method of any one of embodiments 341-345, wherein the clearance rate is determined in a preclinical species or in a human subject.

Embodiment 347: The method of any one of embodiments 341-345, wherein the clearance rate is from noncompartmental analysis using measured inhibitor concentrations over time in one or more blood samples.

Embodiment 348: The method of any one of embodiments 341-345, wherein the clearance rate is determined in blood from preclinical species and allometrically scaled to human.

Embodiment 349: The method of any one of embodiments 341-348, wherein the clearance rate is determined by applying PK simulation and modeling approaches to estimate clearance based on in silico human physiological conditions.

Embodiment 350: The method of any one of embodiments 314-349, wherein the intermittent dosing regimen comprises one dosing day followed by at least one day without dosing.

Embodiment 351: The method of any one of embodiments 314-349, wherein the intermittent dosing regimen comprises at least two consecutive dosing days followed by at least one day without dosing.

Embodiment 352: The method of any one of embodiments 314-349, wherein the intermittent dosing regimen comprises at least three consecutive dosing days followed by at least one day without dosing.

Embodiment 353: The method of any one of embodiments 314-349, wherein the intermittent dosing regimen comprises at least four consecutive dosing days followed by at least one day without dosing.

Embodiment 354: The method of any one of embodiments 314-349, wherein the intermittent dosing regimen comprises at least five consecutive dosing days followed by at least one day without dosing.

Embodiment 355: The method of any one of embodiments 314-349, wherein the intermittent dosing regimen comprises at least six consecutive dosing days followed by at least one day without dosing.

Embodiment 356: The method of any one of embodiments 314-349, wherein each dosing regimen comprises five dosing days and two days without dosing.

Embodiment 357: The method of any one of embodiments 314-349, wherein each dosing regimen comprises four dosing days and three days without dosing.

Embodiment 358: The method of any one of embodiments 314-349, wherein each dosing regimen comprises three dosing days and four days without dosing.

Embodiment 359: The method of any one of embodiments 314-349, wherein each dosing regimen comprises two dosing days and five days without dosing.

Embodiment 360: The method of any one of embodiments 314-349, wherein each dosing regimen comprises one dosing day and six days without dosing.

Embodiment 361: The method of any one of embodiments 314-349, wherein each dosing regimen comprises seven consecutive days of dosing (e.g., Q2W).

Embodiment 362: The method of any one of embodiments 314-349, wherein the RAS(ON) multi-selective inhibitor is administered Q2D.

Embodiment 363: The method of any one of embodiments 314-349, wherein the intermittent dosing regimen is repeated.

Embodiment 364: The method of any one of embodiments 314-349, wherein the daily dose of the RAS(ON) multi-selective inhibitor administered is between 10 mg to 2000 mg.

Embodiment 365: The method of embodiment 364, wherein the daily dose of the RAS(ON) multi-selective inhibitor is administered once per day.

Embodiment 366: The method of embodiment 365, wherein the daily dose of the RAS(ON) multi-selective inhibitor is divided equally into twice per day.

Embodiment 367: The method of any one of embodiments 314-366, wherein the RAS(ON) multi-selective inhibitor is RMC-6236 (daraxonrasib), RMC-7977, Compound A, Compound 6A of WO 2024067857, or ERAS-0015.

Embodiment 368: The method of any one of embodiments 314-367, wherein the RAS(ON) multi-selective inhibitor exposure in normal tissues is decreased during non-dosing days while maintaining antitumor activity.

Embodiment 369: The method of any one of embodiments 314-368, wherein the subject does not exhibit a dose limiting toxicity.

Embodiment 370: The method of any one of embodiments 314-369, wherein the frequency or severity of treatment related adverse events are reduced compared to the frequency or severity of treatment related events of daily administration of the RAS(ON) multi-selective inhibitor.

Embodiment 371: The method of embodiment 370, wherein the treatment related adverse event is an on-target RAS-pathway mediated toxicity.

Embodiment 372: The method of embodiment 370 or 371, wherein the treatment related adverse event is rash or a GI-related toxicity.

Embodiment 373: A method of selecting a dosing regimen for a RAS(ON) multi-selective inhibitor, the method comprising: determining the K_{D}1 of the RAS(ON) multi-selective inhibitor to CypA; and selecting a RAS(ON) multi-selective inhibitor for intermittent administration when the K_{D}1 is less than 500 nM.

Embodiment 374: The method of embodiment 373, wherein the method further comprises administering the RAS(ON) multi-selective inhibitor using an intermittent dosing regimen.

Embodiment 375: A pharmaceutical composition comprising a RAS(ON) multi-selective inhibitor of the disclosure and a pharmaceutically acceptable carrier for use in the method of any one of the embodiments 1-374.

Embodiment 376: Use of a RAS(ON) multi-selective compound of the disclosure in the manufacture of a medicament for use in the method of any one of the embodiments 1-374.

### EXAMPLES

The disclosure is further illustrated by the following example, which is not to be construed as limiting this disclosure in scope or spirit to the specific procedures herein described. It is to be understood that the example is provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1—Intermittent Dosing of RAS(ON) Multi-Selective Inhibitors

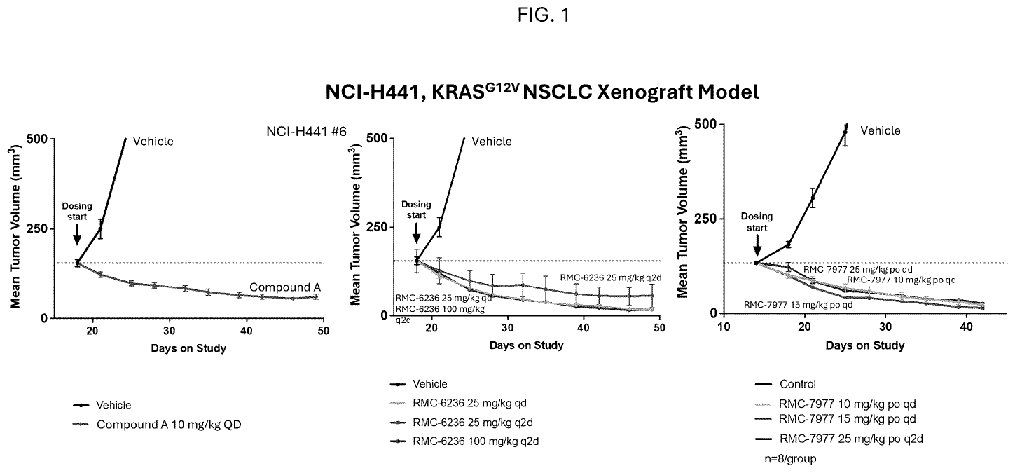

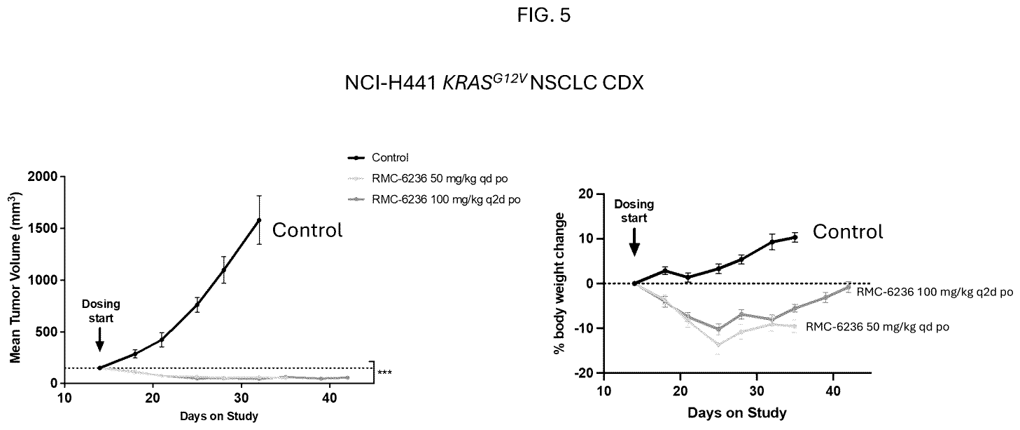

Intermittent dosing has been associated with reduced frequency and less severe AEs than the daily dosing schedule of the drug on the RAS signaling pathways. In preclinical models, RMC-6236 trends to exhibit more severe body weight loss and intolerability via BID dosing, whereas Q2D dosing slightly improves the body weight loss at an equivalent dose of QD dosing, and also allows higher dose levels to be tolerated. In experimental results indicated in (FIG. 1), RMC-6236 was dosed in a preclinical efficacy study at 25 mg/kg (QD), 25 mg/kg (Q2D) and 100 mg/kg (Q2), and results demonstrated similar decreases in tumor volume when dosed at 25 mg/kg (QD) or 100 mg/kg (Q2D), whereas 25 mg/kg (Q2D) showed lower decreases in tumor volume. In experimental results indicated in (FIG. 5), RMC-6236 was dosed in preclinical efficacy study at 10 mg/kg (QD), 20 mg/kg (QD), 20 mg/kg (Q2D) and 40 mg/kg (Q2D), and body weight measurements showed similar effects at all dose levels and schedules.

### Methods

All mouse studies and procedures related to animal handling, care and treatment complied with all applicable regulations and guidelines of Institutional Animal Care and Use Committee (IACUC) approvals. Female BALB/c nude mice, 6-8 weeks old were used. Animal vendor was Shanghai Sino-British Sippr/BK Laboratory Animal Co., LTD. NCI-H441 tumor cells (ATCC-HTB-174) was maintained in vitro in RPMI-1640 medium supplemented with 10% heat inactivated FBS, 100 μg/mL streptomycin. Each mouse was inoculated subcutaneously at the right flank with 2×10⁶ tumor cells in 0.2 ml media/matrigel for tumor development. Efficacy treatment started when the average tumor size reached 150-200 mm³ in size, and the single dosed PKPD treatment started when tumor size reached 300-600 mm³. Mice in the study were weighted and tumors were measured twice weekly.

NCI-H441 is a human lung tumor derived cell line. It harbors a KRAS^{G12V} mutation. It is typically grown in immune-deficient mice as a xenograft model. An NCI-H441 xenograft study was conducted in female BALB/C Nude mice. Each mouse was inoculated subcutaneously on the right flank with the single cell suspension of tumor cells (2×10⁶) in 200 uL with BD Matrigel (1:1 ratio) for tumor development. Tumor width and length were measured with digital calipers, and the tumor volume in mm3 was calculated using the formula: Volume=((width)2×length)/2, where width and length are measured in mm.

Tumor growth inhibition (TGI, %) was calculated as percentage change in tumor volume for each group 100×(Vday_{xc}−Vdayₓ)/(Vday_{xc}−Vday_{oc}), where Vday_{0c} is the starting mean tumor volume for the vehicle group, and Vdayₓ and Vday_{xc} are the corresponding tumor volumes for treatment group and control group on day x, calculated for the duration of the study. Mean tumor regression (where applicable, Vdayₓ<Vday₀ₓ) was calculated using the formula 100×(Vday₀ₓ−Vdayₓ)/Vday₀ₓ.

Differences in treatment arms as compared to vehicle controls were assessed by an ordinary one-way ANOVA of tumor volumes along with multiple comparisons via a post-hoc Tukey's test in GraphPad Prism software. Note, in some studies, additional test articles/groups (not reported here) were included and statistical analyses (ANOVA, post-hoc within group comparisons) were conducted on all treatment groups to allow for any interactions. The statistical significance values reported here represent the output of such analyses for RMC-6236 treatment arm.

Capan-2 is a human pancreatic adenocarcinoma derived cell line. It harbors a KRAS^{G12V} mutation. It is typically grown in immune-deficient mice as a xenograft model. A Capan-2 xenograft study was conducted in female BALB/C Nude mice. Each mouse was inoculated subcutaneously on the right flank with the single cell suspension of tumor cells (4×10⁶) in 100 uL RPMI 1640/Matrigel mixture (1:1 ratio) for tumor development. Tumor width and length were measured with digital calipers, and the tumor volume in mm3 was calculated using the formula: Volume=((width)2×length)/2, where width and length are measured in mm. Tumor growth inhibition (TGI, %) was calculated as percentage change in tumor volume for each group 100×(Vday_{xc}−Vdayₓ)/(Vday_{xc}−Vday_{0c}), where Vday_{0c} is the starting mean tumor volume for the vehicle group, and Vdayₓ and Vday_{xc} are the corresponding tumor volumes for treatment group and control group on day x, calculated for the duration of the study. Mean tumor regression (where applicable, Vdayₓ<Vday₀ₓ) was calculated using the formula 100×(Vday₀ₓ−Vdayₓ)/Vday₀ₓ.

### Results

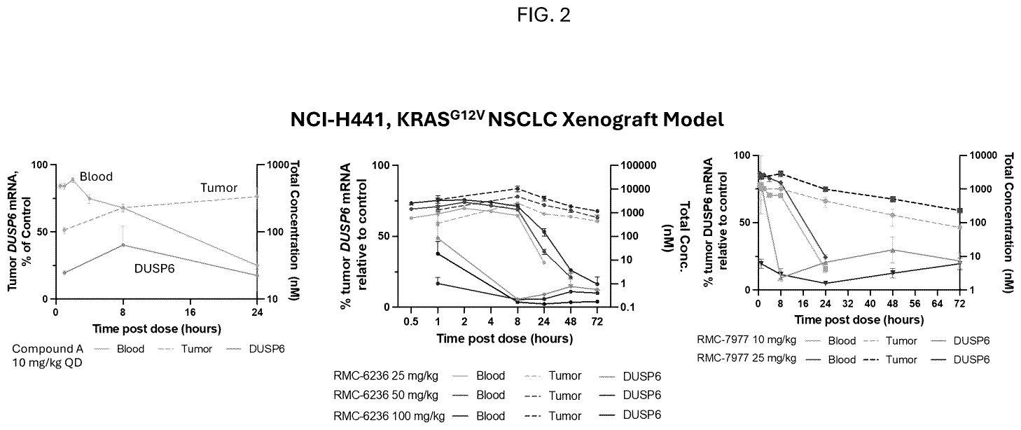

Anti-tumor activity was observed by several different RAS(ON) multi-selective compounds in the NCI-H441 KRAS^{G12V} NSCLC xenograft model. RMC-034, RMC-6236 and RMC-7977 were able to drive similar tumor regression at different doses (FIG. 1). The pharmacokinetics and pharmacodynamics profiles of 3 compounds were also assessed in the NCI-H441 model. Prolonged tumor exposure was observed for all three compounds at doses ranging between 10 mg/kg up to 100 mg/kg, while the blood exposure fell below detection limit after 24 hr for most of the doses. Consistent with the prolonged exposure in the NCI-H441 xenograft tumor, oral administration of three compounds also led to durable suppression human DUSP6 as a marker of the RAS pathway suppression. RMC-6236 and RMC-7977 induced maximum suppression at 8 hr post single dose and had durable suppression up to 72 hr, while Compound A induced maximum suppression at a later time point at 24 hr (FIG. 2).

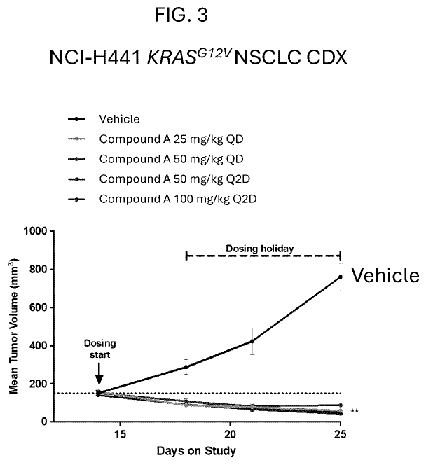

Compound A anti-tumor activity was further examined with various doses in FIG. 3. Compound A 25 mg/kg daily and 50 mg/kg daily, along with their exposure equivalent of Compound A 50 mg/kg Q2D and Compound A 100 mg/kg Q2D, induced the same tumor regression though all treatment groups require some dosing holidays towards the end.

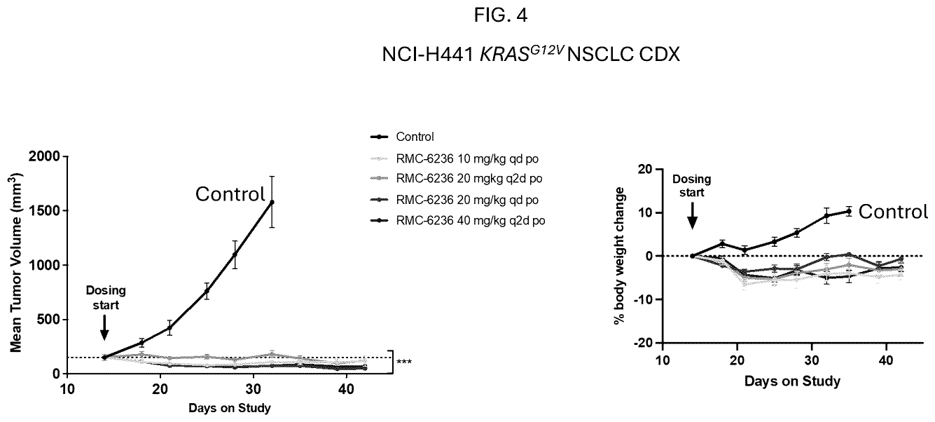

The difference in anti-tumor activity between daily dosing versus intermittent dosing was also assessed with RMC-6236. Similar tumor regression was obtained between 10 m/kg QD vs 20 mg/kg Q2D, 20 mg/kg QD and 40 mg/kg Q2D, and 50 mg/kg QD and 100 mg/kg Q2D (FIG. 4 and FIG. 5). All regimens tested here were tolerated in vivo.

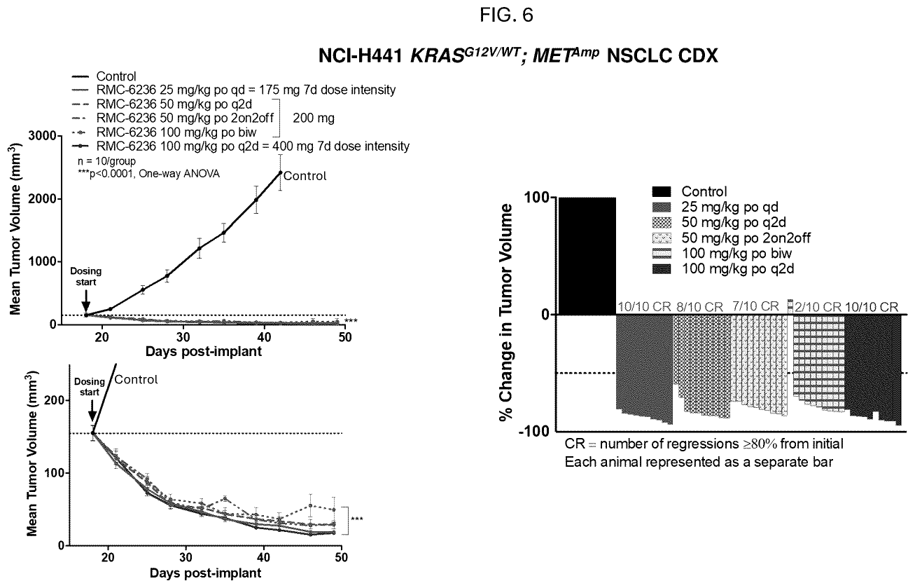

In the experiments shown in FIG. 6, 25 mg/kg QD and 100 mg/kg Q2D of RMC-6236 gave the best tumor regression of 88% and 89% in the NCI-H441 model. With increased interval of dosing, a tumor breakthrough evidenced by 68% tumor regression was observed by 100 mpk of RMC-6236, which was significantly lower than the two most efficacious doses. All doses and regimens were tolerated in this study without any significant body weight loss.

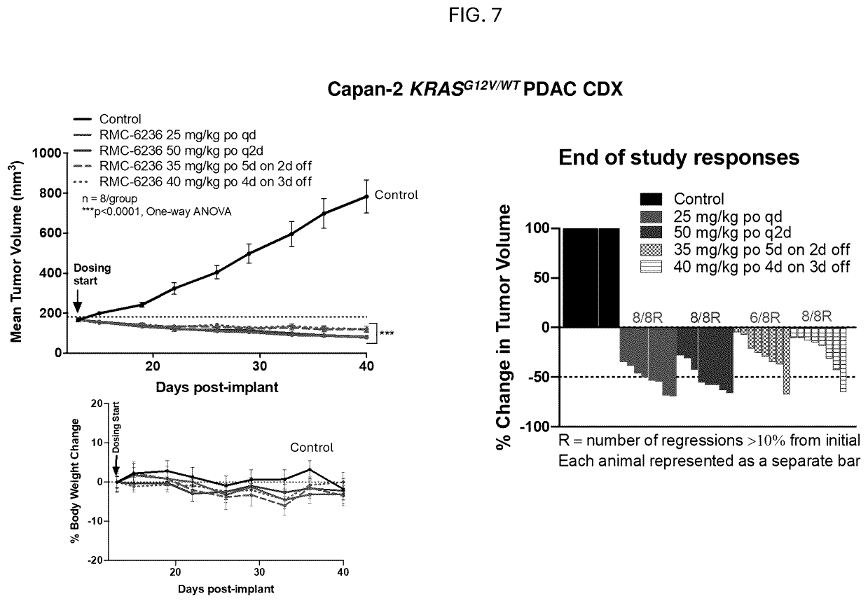

Tumor growth inhibition was observed at all tested doses and regimens of RMC-6236 by oral administration in the Capan-2 human adenocarcinoma xenograft model (FIG. 7). Tumor volume graph showed that best tumor regression was observed in the 25 mg/kg QD and 50 mg/kg Q2D groups, as evidenced by 53% and 51% tumor regression respectively. The alternate dosing groups of 35 mg/kg 5on2off and 40 mg/kg 4on3off had lower % tumor regression of 28% and 27%, indicative of increased tumor breakthrough in these alternative dosing regimens. As demonstrated by the body weight graph, tumor bearing mice tolerated all tested doses and regimens of RMC-6236 well, without any significant body weight loss.

The half-life of RMC-6236 after oral administration to patients was about 10 hours, which was within the range of those predicted from preclinical PK studies. Longer half-life (e.g., >24 hours) RMC-6236-like RAS inhibitors are expected to be accumulated at steady state, and potentially accumulated in normal tissues with even a longer half-life. The in vitro properties of RMC-7977, RMC-6236, and Compound A are summarized in Table 3.

TABLE 3
In vitro Properties of RMC-7977, RMC-6236, and Compound A

| | | RMC- | RMC- | Compound |
|---|---|---|---|---|
| | | 6236 | 7977 | A |
| RAS-RAF | KRAS MUT | 35-229 | 31-175 | 23-165 |
| Disruption (IC₅₀, | (G12C, G12D, | | | |
| nM) | G12V) | | | |
| | KRAS WT | 92 | 76 | 42 |
| | NRAS MUT | 57-204 | 48-144 | 18-109 |
| | (Q61H, Q61K, | | | |
| | Q61L, Q61R) | | | |
| | NRAS WT | 72 | 57 | 33 |
| CypA Binding | | 57 | 216 | 12 |
| (K_{D}1, nM) | | | | |
| KRAS Binding of | KRAS MUT | 131-364 | 111-435 | |
| CypA:TCI (K_{D}2, nM) | (G12D, G12V) | | | |
| | KRAS WT | 157 | 116 | 81 |
| Cellular pERK | KRAS MUT | 0.4-3.6 | 0.5-4.1 | 0.3-1.0 |
| (2D, 4 hr) (IC₅₀, | (G12C, G12D, | | | |
| nM) | G12V) | | | |
| | RAS WT | 1.5 | 1.7 | 0.4 |
| 2D Cell Viability, | KRAS MUT | 1.0-3.1 | 0.8-4.9 | 0.2-1.2 |
| 120 hr (IC₅₀, nM) | (G12C, G12D, | | | |
| | G12V) | | | |
| | BRAF (V600E) | 7212 | 8365 | 1881 |
| 3D Cell Viability, | RAS WT | 1.4 | 1.9 | 0.4 |
| 120 hr (IC₅₀, nM) | | | | |

| | | | |
|---|---|---|---|
| Oral % F (multiple species) | 24-33 | 60 | 20-38 |

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent or patent application was specifically and individually indicated to be incorporated by reference in its entirety.

1. A method of treating a RAS protein-related disease in a subject in need thereof, the method comprising administering to the subject a RAS(ON) multi-selective inhibitor on an intermittent dosing regimen.

2. The method of claim 1, wherein the subject has a mutation of RAS.

3. The method of claim 1 or claim 2, wherein the RAS protein-related disease is cancer.

4. The method of any one of claims 1 to 3, wherein the RAS(ON) multi-selective inhibitor has a blood or plasma half-life of 12 hours or longer.

5. The method of any one of claims 1 to 4, wherein the RAS(ON) multi-selective inhibitor has a K_{D}1 of 0.1 nM to 500 nM.

6. The method of any one of claims 1 to 5, wherein the RAS(ON) multi-selective inhibitor has a clearance rate of 0.4 L/h/kg or slower.

7. The method of any one of claims 1 to 6, wherein the intermittent dosing regimen comprises one dosing day followed by at least one day without dosing.

8. The method of any one of claims 1 to 6, wherein the intermittent dosing regimen comprises at least two consecutive dosing days followed by at least one day without dosing.

9. The method of any one of claims 1 to 6, wherein the intermittent dosing regimen comprises at least three consecutive dosing days followed by at least one day without dosing.

10. The method of any one of claims 1 to 6, wherein the intermittent dosing regimen comprises at least four consecutive dosing days followed by at least one day without dosing.

11. The method of any one of claims 1 to 6, wherein the intermittent dosing regimen comprises at least five consecutive dosing days followed by at least one day without dosing.

12. The method of any one of claims 1 to 6, wherein the intermittent dosing regimen comprises at least six consecutive dosing days followed by at least one day without dosing.

13. The method of any one of claims 1 to 6, wherein each dosing regimen comprises five dosing days and two days without dosing.

14. The method of any one of claims 1 to 6, wherein each dosing regimen comprises four dosing days and three days without dosing.

15. The method of any one of claims 1 to 6, wherein each dosing regimen comprises three dosing days and four days without dosing.

16. The method of any one of claims 1 to 6, wherein each dosing regimen comprises two dosing days and five days without dosing.

17. The method of any one of claims 1 to 6, wherein each dosing regimen comprises one dosing day and six days without dosing.

18. The method of any one of claims 1 to 6, wherein the RAS(ON) multi-selective inhibitor is administered Q2D.

19. The method of any one of claims 1 to 18, wherein the intermittent dosing regimen is repeated.

20. The method of any one of claims 1 to 19, wherein the dosing regimen comprises administering the RAS(ON) multi-selective inhibitor and an additional therapeutic agent.

21. The method of any one of claims 1 to 20, wherein the RAS(ON) multi-selective inhibitor is 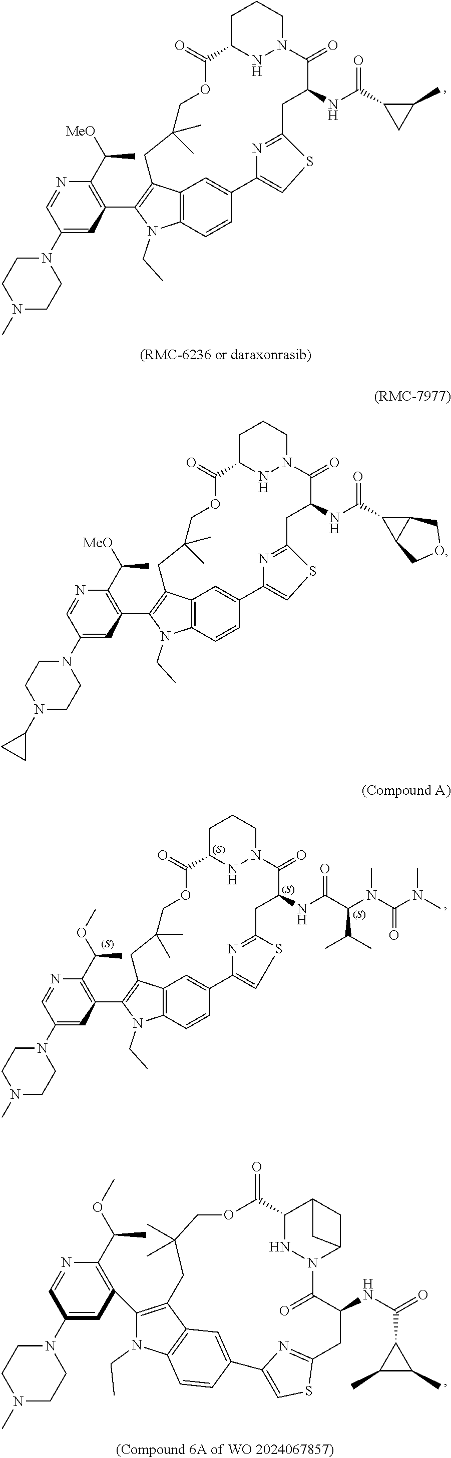
or ERAS-0015.

22. The method of claim 20 or 21, wherein the additional therapeutic agent is a RAS(OFF) inhibitor.

23. The method of any one of claims 20 to 22, wherein the additional therapeutic agent is a pan-KRAS inhibitor.

24. The method of claim 23, wherein the pan-KRAS inhibitor is ERAS-4001.

25. A method of treating a RAS protein-related disease or disorder comprising administering to a subject in need thereof a RAS(ON) multi-selective inhibitor and an additional RAS inhibitor, wherein the RAS(ON) multi-selective inhibitor is administered on an intermittent dosing regimen.

26. The method of claim 25, wherein the additional RAS inhibitor is administered on a daily dosing regimen or on an intermittent dosing regimen.

27. The method of claim 25 or 26, wherein the additional RAS inhibitor is a RAS(OFF) inhibitor.

28. The method of any one of claims 25 to 27, wherein the additional RAS inhibitor is a pan-KRAS inhibitor.

29. The method of claim 28, wherein the pan-KRAS inhibitor is ERAS-4001.

30. The method of any one of claims 25 to 29, wherein the subject has a RAS mutation.

31. The method of any one of claims 25 to 30, wherein the RAS(ON) multi-selective inhibitor has a blood or plasma half-life of 12 hours or longer.

32. The method of any one of claims 25 to 31, wherein the RAS(ON) multi-selective inhibitor has a K_{D}1 of 0.1 nM to 500 nM.

33. The method of any one of claims 25 to 32, wherein the RAS(ON) multi-selective inhibitor has a clearance rate of 0.4 L/h/kg or slower.

34. The method of any one of claims 25 to 32, wherein the intermittent dosing regimen comprises one dosing day followed by at least one day without dosing.

35. The method of any one of claims 25 to 34, wherein the RAS(ON) multi-selective inhibitor is administered Q2D.

36. The method of any one of claims 25 to 35, wherein the RAS(ON) multi-selective inhibitor 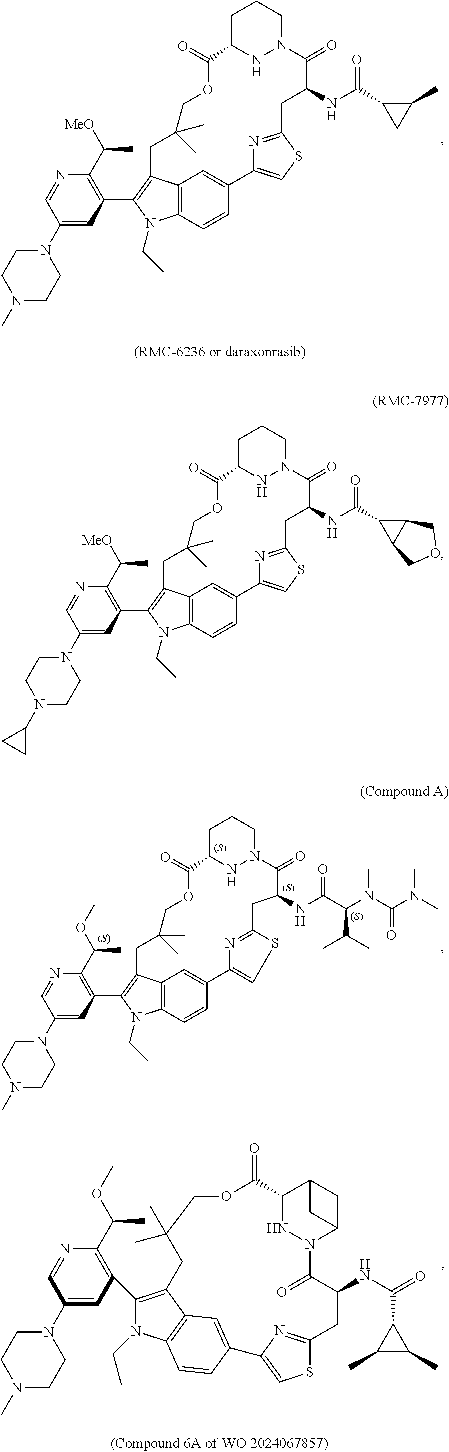
or ERAS-0015.
